SEQUENCE LISTING (263 sequences)

SEQ ID NO: 1 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 2 - protein, 193 aa, Macaca sp.
MAAEPAEDNCINFVAMKPIDSTLYFIAEDDENLESDYFGKLESKLSIIRNLNDQVLFIDQGNRPLFEDMTDSDCRDNAPRTIFIINMYKDSQPRGMAVAISVKCEKISTLSCENRIISFKEMNPPDNIKDTKSDIIFFQRSVPGHDNKMQFESSSYEGYFLACEKERDLYKLILKKKDELGDRSIMFTVQNED

SEQ ID NO: 3 - protein, 5 aa, Artificial Sequence
  Description of Artificial Sequence Synthetic peptide
SYAIS

SEQ ID NO: 4 - protein, 17 aa, Artificial Sequence
  Description of Artificial Sequence Synthetic peptide
GIIPIYGTANYAQKFQG

SEQ ID NO: 5 - protein, 10 aa, Artificial Sequence
  Description of Artificial Sequence Synthetic peptide
AAYHPLVFDN

SEQ ID NO: 6 - protein, 13 aa, Artificial Sequence
  Description of Artificial Sequence Synthetic peptide
SGSSSNIGNHYVN

SEQ ID NO: 7 - protein, 7 aa, Artificial Sequence
  Description of Artificial Sequence Synthetic peptide
RNNHRPS

SEQ ID NO: 8 - protein, 11 aa, Artificial Sequence
  Description of Artificial Sequence Synthetic peptide
QSWDYSGFSTV

SEQ ID NO: 9 - protein, 17 aa, Artificial Sequence
  Description of Artificial Sequence Synthetic peptide
NIIPMTGQTYYAQKFQG

SEQ ID NO: 10 - protein, 18 aa, Artificial Sequence
  Description of Artificial Sequence Synthetic peptide
WINPFYIGETFYAQKFQG

SEQ ID NO: 11 - protein, 17 aa, Artificial Sequence
  Description of Artificial Sequence Synthetic peptide
NIIPHYGFAYYAQKFQG

SEQ ID NO: 12 - protein, 17 aa, Artificial Sequence
  Description of Artificial Sequence Synthetic peptide
NIIPYSGFAYYAQKFQG

SEQ ID NO: 13 - protein, 17 aa, Artificial Sequence
  Description of Artificial Sequence Synthetic peptide
NIIPITGQTYYAQKFQG

SEQ ID NO: 14 - protein, 119 aa, Artificial Sequence
  Description of Artificial Sequence Synthetic polypeptide
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSS

SEQ ID NO: 15 - DNA, 357 bp, Artificial Sequence
  Description of Artificial Sequence Synthetic polynucleotide
gaggtgcagctggtgcagtcaggcgccgaagtgaagaaacccggctctagcgtgaaagtcagctgtaaagctagtggcggcaccttcaagtcctacgctattagctgggtcagacaggccccaggtcagggcctggagtggatgggcaatattatccctatgaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagc

SEQ ID NO: 16 - protein, 110 aa, Artificial Sequence
  Description of Artificial Sequence Synthetic polypeptide
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVL

SEQ ID NO: 17 - DNA, 330 bp, Artificial Sequence
  Description of Artificial Sequence Synthetic polynucleotide
gatatcgtcctgactcagccccctagcgtcagcggcgctcccggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcaggcactagcgctagtctggctatcaccggactgcagtcagaggacgaggccgactactactgtcagtcctgggactatagcggctttagcaccgtgttcggcggaggcactaagctgaccgtgctg

SEQ ID NO: 18 - protein, 119 aa, Artificial Sequence
  Description of Artificial Sequence Synthetic polypeptide
QVQLVQSGAEVKKPGSSVKVSCKASGGTFSSYAISWVRQAPGQGLEWMGNIIPITGQTYYAQKFQGRVTITADESTSTAYMELSSLRSEDTAVYYCARAAYHPLVFDNWGQGTLVTVSS

SEQ ID NO: 19 - DNA, 357 bp, Artificial Sequence
  Description of Artificial Sequence Synthetic polynucleotide
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcaatattatccctatcaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagc

SEQ ID NO: 20 - protein, 110 aa, Artificial Sequence
  Description of Artificial Sequence Synthetic polypeptide
QSVLTQPPSASGTPGQRVTISCSGSSSNIGNHYVNWYQQLPGTAPKLLIYRNNHRPSGVPDRFSGSKSGTSASLAISGLQSEDEADYYCQSWDYSGFSTVFGGGTKLTVL

SEQ ID NO: 21 - DNA, 330 bp, Artificial Sequence
  Description of Artificial Sequence Synthetic polynucleotide
cagtcagtcctgactcagccccctagcgctagtggcacccctggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcagggactagcgctagtctggctattagcggcctgcagtcagaggacgaggccgactactactgtcagtcctgggactatagcggctttagcaccgtgttcggcggaggcactaagctgaccgtgctg

SEQ ID NO: 22 - protein, 119 aa, Artificial Sequence
  Description of Artificial Sequence Synthetic polypeptide
QVQLVQSGAEVKKPGSSVKVSCKASGGTFNSYAISWVRQAPGQGLEWMGGIIPIYGTANYAQKFQGRVTITADESTSTAYMELSSLRSEDTAVYYCARAAYHPLVFDNWGQGTLVTVSS

SEQ ID NO: 23 - DNA, 357 bp, Artificial Sequence
  Description of Artificial Sequence Synthetic polynucleotide
caggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcggtatcattccgatttatggcactgcgaattacgcgcagaagtttcagggccgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctca

SEQ ID NO: 24 - DNA, 330 bp, Artificial Sequence
  Description of Artificial Sequence Synthetic polynucleotide
gatatcgtgctgacccagccgccttcagtgagtggcgcaccaggtcagcgtgtgaccatctcgtgtagcggcagcagcagcaacattggtaatcattatgtgaattggtaccagcagttgcccgggacggcgccgaaacttctgatttatcgtaataatcatcgtccctcaggcgtgccggatcgttttagcggatccaaaagcggcaccagcgcgagccttgcgattacgggcctgcaaagcgaagacgaagcggattattattgccagtcttgggattattctggtttttctactgtgtttggcggcggcacgaagttaaccgtccta

SEQ ID NO: 25 - protein, 120 aa, Artificial Sequence
  Description of Artificial Sequence Synthetic polypeptide
EVQLVQSGAEVKKPGSSVKVSCKASGGTFNSYAISWVRQAPGQGLEWMGWINPFYIGETFYAQKFQGRVTITADESTSTAYMELSSLRSEDTAVYYCARAAYHPLVFDNWGQGTLVTVSS

SEQ ID NO: 26 - DNA, 360 bp, Artificial Sequence
  Description of Artificial Sequence Synthetic polynucleotide
gaggtgcagctggtgcagtctggcgctgaggtgaagaagcctggctcctccgtcaaggtgtcctgcaaggcctccggcggcaccttcaactcctacgctatctcttgggtgcgccaggctcccggacagggcctggagtggatgggctggatcaaccctttctacatcggcgagacattctacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctcc

SEQ ID NO: 27 - DNA, 330 bp, Artificial Sequence
  Description of Artificial Sequence Synthetic polynucleotide
gatatcgtgctgacccagcctccttctgtgtctggcgcccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctctgcctccctggccatcaccggcctgcagtccgaggacgaggccgactactactgccagtcctgggactactccggcttctcaaccgtgttcggcggaggcaccaagctgaccgtgctg

SEQ ID NO: 28 - protein, 119 aa, Artificial Sequence
  Description of Artificial Sequence Synthetic polypeptide
EVQLVQSGAEVKKPGSSVKVSCKASGGTFNSYAISWVRQAPGQGLEWMGNIIPMTGQTYYAQKFQGRVTITADESTSTAYMELSSLRSEDTAVYYCARAAYHPLVFDNWGQGTLVTVSS

SEQ ID NO: 29 - DNA, 357 bp, Artificial Sequence
  Description of Artificial Sequence Synthetic polynucleotide
gaggtgcagctggtgcagtctggcgctgaggtgaagaagcctggctcctccgtcaaggtgtcctgcaaggcctccggcggcaccttcaactcctacgctatctcttgggtgcgccaggctcccggacagggcctggagtggatgggcaacatcatccctatgaccggccagacctactacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctcc

SEQ ID NO: 30 - DNA, 330 bp, Artificial Sequence
  Description of Artificial Sequence Synthetic polynucleotide
gacatcgtgctgacacagcctccctctgtgtctggcgcccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctctgcctccctggccatcaccggcctgcagtcagaggacgaggccgactactactgccagtcctgggactactccggcttctccaccgtgttcggcggaggcaccaagctgaccgtgctg

SEQ ID NO: 31 - protein, 119 aa, Artificial Sequence
  Description of Artificial Sequence Synthetic polypeptide
EVQLVQSGAEVKKPGSSVKVSCKASGGTFNSYAISWVRQAPGQGLEWMGNIIPHYGFAYYAQKFQGRVTITADESTSTAYMELSSLRSEDTAVYYCARAAYHPLVFDNWGQGTLVTVSS

SEQ ID NO: 32 - DNA, 357 bp, Artificial Sequence
  Description of Artificial Sequence Synthetic polynucleotide
gaggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcaatattattcctcattatggttttgcttattatgctcagaagtttcagggtcgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctca

SEQ ID NO: 33 - DNA, 330 bp, Artificial Sequence; identical to SEQ ID NO: 24
  Description of Artificial Sequence Synthetic polynucleotide

SEQ ID NO: 34 - protein, 119 aa, Artificial Sequence
  Description of Artificial Sequence Synthetic polypeptide
EVQLVQSGAEVKKPGSSVKVSCKASGGTFNSYAISWVRQAPGQGLEWMGNIIPYSGFAYYAQKFQGRVTITADESTSTAYMELSSLRSEDTAVYYCARAAYHPLVFDNWGQGTLVTVSS

SEQ ID NO: 35 - DNA, 357 bp, Artificial Sequence
  Description of Artificial Sequence Synthetic polynucleotide
gaggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcaatattattccttattctggttttgcttattatgctcagaagtttcagggtcgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctca

SEQ ID NO: 36 - DNA, 330 bp, Artificial Sequence; identical to SEQ ID NO: 24
  Description of Artificial Sequence Synthetic polynucleotide

SEQ ID NO: 37 - protein, 120 aa, Artificial Sequence; identical to SEQ ID NO: 25
  Description of Artificial Sequence Synthetic polypeptide

SEQ ID NO: 38 - DNA, 360 bp, Artificial Sequence; identical to SEQ ID NO: 26
  Description of Artificial Sequence Synthetic polynucleotide

SEQ ID NO: 39 - DNA, 330 bp, Artificial Sequence
  Description of Artificial Sequence Synthetic polynucleotide
cagtccgtgctgacccagcctccttctgcctctggcacccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctccgcctccctggccatctctggcctgcagtcagaggacgaggccgactactactgccagtcctgggactactccggcttctccaccgtgttcggcggaggcaccaagctgaccgtgctg

SEQ ID NO: 40 - protein, 119 aa, Artificial Sequence; identical to SEQ ID NO: 28
  Description of Artificial Sequence Synthetic polypeptide

SEQ ID NO: 41 - DNA, 357 bp, Artificial Sequence
  Description of Artificial Sequence Synthetic polynucleotide
gaggtgcagctggtgcagtctggcgccgaggtgaagaagcctggctcctccgtcaaggtgtcctgcaaggcctccggcggcaccttcaactcctacgccatctcttgggtgcgccaggctcctggacagggcctggagtggatgggcaacatcatccctatgaccggccagacctactacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctcc

SEQ ID NO: 42 - DNA, 330 bp, Artificial Sequence; identical to SEQ ID NO: 39
  Description of Artificial Sequence Synthetic polynucleotide

SEQ ID NO: 43 - protein, 449 aa, Artificial Sequence
  Description of Artificial Sequence Synthetic polypeptide
EVQLVQSGAEVKKPGSSVKVSCKASGGTFKSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - DNA, 1347 bp, Artificial Sequence
  Description of Artificial Sequence Synthetic polynucleotide
gaggtgcagctggtgcagtcaggcgccgaagtgaagaaacccggctctagcgtgaaagtcagctgtaaagctagtggcggcaccttcaagtcctacgctattagctgggtcagacaggccccaggtcagggcctggagtggatgggcaatattatccctatgaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 45 - protein, 216 aa, Artificial Sequence
  Description of Artificial Sequence Synthetic polypeptide
DIVLTQPPSVSGAPGQRVTISCSGSSSNIGNHYVNWYQQLPGTAPKLLIYRNNHRPSGVPDRFSGSKSGTSASLAITGLQSEDEADYYCQSWDYSGFSTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 46 - DNA, 648 bp, Artificial Sequence
  Description of Artificial Sequence Synthetic polynucleotide
gatatcgtcctgactcagccccctagcgtcagcggcgctcccggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcaggcactagcgctagtctggctatcaccggactgcagtcagaggacgaggccgactactactgtcagtcctgggactatagcggctttagcaccgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 47 - protein, 449 aa, Artificial Sequence
  Description of Artificial Sequence Synthetic polypeptide
EVQLVQSGAEVKKPGSSVKVSCKASGGTFNSYAISWVRQAPGQGLEWMGNIIPMTGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 1347 bp, Artificial Sequence
  Description of Artificial Sequence Synthetic polynucleotide
gaggtgcagctggtgcagtctggcgctgaggtgaagaagcctggctcctccgtcaaggtgtcctgcaaggcctccggcggcaccttcaactcctacgctatctcttgggtgcgccaggctcccggacagggcctggagtggatgggcaacatcatccctatgaccggccagacctactacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctccgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 49 - DNA, 648 bp, Artificial Sequence
  Description of Artificial Sequence Synthetic polynucleotide
gacatcgtgctgacacagcctccctctgtgtctggcgcccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctctgcctccctggccatcaccggcctgcagtcagaggacgaggccgactactactgccagtcctgggactactccggcttctccaccgtgttcggcggaggcaccaagctgaccgtgctgggacagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 50 - protein, 450 aa, Artificial Sequence
  Description of Artificial Sequence Synthetic polypeptide
EVQLVQSGAEVKKPGSSVKVSCKASGGTFNSYAISWVRQAPGQGLEWMGWINPFYIGETFYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - DNA, 1350 bp, Artificial Sequence
  Description of Artificial Sequence Synthetic polynucleotide
gaggtgcagctggtgcagtctggcgctgaggtgaagaagcctggctcctccgtcaaggtgtcctgcaaggcctccggcggcaccttcaactcctacgctatctcttgggtgcgccaggctcccggacagggcctggagtggatgggctggatcaaccctttctacatcggcgagacattctacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctccgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 52 - DNA, 648 bp, Artificial Sequence
  Description of Artificial Sequence Synthetic polynucleotide
gatatcgtgctgacccagcctccttctgtgtctggcgcccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctctgcctccctggccatcaccggcctgcagtccgaggacgaggccgactactactgccagtcctgggactactccggcttctcaaccgtgttcggcggaggcaccaagctgaccgtgctgggacagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 53 - protein, 449 aa, Artificial Sequence; identical to SEQ ID NO: 47
  Description of Artificial Sequence Synthetic polypeptide

SEQ ID NO: 54 - DNA, 1347 bp, Artificial Sequence
  Description of Artificial Sequence Synthetic polynucleotide
gaggtgcagctggtgcagtctggcgccgaggtgaagaagcctggctcctccgtcaaggtgtcctgcaaggcctccggcggcaccttcaactcctacgccatctcttgggtgcgccaggctcctggacagggcctggagtggatgggcaacatcatccctatgaccggccagacctactacgcccagaagttccagggcagagtcaccatcaccgccgacgagtccacctccaccgcctacatggagctgtcctccctgcggtcagaggacaccgccgtgtactactgcgccagggccgcctaccaccctctggtgttcgacaactggggccagggcaccctggtgaccgtgtcctccgctagcaccaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 55 - DNA, 648 bp, Artificial Sequence
  Description of Artificial Sequence Synthetic polynucleotide
cagtccgtgctgacccagcctccttctgcctctggcacccctggccagagagtgaccatctcctgctctggctcctcctccaatatcggcaaccactacgtgaactggtatcagcagctgcccggaaccgcccctaagctgctgatctaccggaacaaccaccggccttccggcgtgcccgaccggttctccggctccaagtctggcacctccgcctccctggccatctctggcctgcagtcagaggacgaggccgactactactgccagtcctgggactactccggcttctccaccgtgttcggcggaggcaccaagctgaccgtgctgggacagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 56 - protein, 449 aa, Artificial Sequence
  Description of Artificial Sequence Synthetic polypeptide
QVQLVQSGAEVKKPGSSVKVSCKASGGTFNSYAISWVRQAPGQGLEWMGGIIPIYGTANYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1347 bp, Artificial Sequence
  Description of Artificial Sequence Synthetic polynucleotide
caggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcggtatcattccgatttatggcactgcgaattacgcgcagaagtttcagggccgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 58 - DNA, 648 bp, Artificial Sequence
  Description of Artificial Sequence Synthetic polynucleotide
gatatcgtgctgacccagccgccttcagtgagtggcgcaccaggtcagcgtgtgaccatctcgtgtagcggcagcagcagcaacattggtaatcattatgtgaattggtaccagcagttgcccgggacggcgccgaaacttctgatttatcgtaataatcatcgtccctcaggcgtgccggatcgttttagcggatccaaaagcggcaccagcgcgagccttgcgattacgggcctgcaaagcgaagacgaagcggattattattgccagtcttgggattattctggtttttctactgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 59 - protein, 7 aa, Artificial Sequence
  Description of Artificial Sequence Synthetic peptide
GGTFNSY

SEQ ID NO: 60 - protein, 6 aa, Artificial Sequence
  Description of Artificial Sequence Synthetic peptide
IPMTGQ

SEQ ID NO: 61 - protein, 10 aa, Artificial Sequence
  Description of Artificial Sequence Synthetic peptide
AAYHPLVFDN

SEQ ID NO: 62 - protein, 9 aa, Artificial Sequence
  Description of Artificial Sequence Synthetic peptide
SSSNIGNHY

SEQ ID NO: 63 - protein, 3 aa, Artificial Sequence
  Description of Artificial Sequence Synthetic peptide
RNN

SEQ ID NO: 64 - protein, 8 aa, Artificial Sequence
  Description of Artificial Sequence Synthetic peptide
WDYSGFST

SEQ ID NO: 65 - protein, 7 aa, Artificial Sequence
  Description of Artificial Sequence Synthetic peptide
GGTFKSY

SEQ ID NO: 66 - protein, 7 aa, Artificial Sequence
  Description of Artificial Sequence Synthetic peptide
GGTFSSY

SEQ ID NO: 67 - protein, 6 aa, Artificial Sequence
  Description of Artificial Sequence Synthetic peptide
IPITGQ

SEQ ID NO: 68 - protein, 7 aa, Artificial Sequence
  Description of Artificial Sequence Synthetic peptide
GFTFSSY

SEQ ID NO: 69 - protein, 6 aa, Artificial Sequence
  Description of Artificial Sequence Synthetic peptide
SGEGSN

SEQ ID NO: 70 - protein, 9 aa, Artificial Sequence
  Description of Artificial Sequence Synthetic peptide
VMIGYGFDY

SEQ ID NO: 71 - protein, 7 aa, Artificial Sequence
  Description of Artificial Sequence Synthetic peptide
SQSIFNY

SEQ ID NO: 72 - protein, 3 aa, Artificial Sequence
  Description of Artificial Sequence Synthetic peptide
DSS

SEQ ID NO: 73 - protein, 6 aa, Artificial Sequence
  Description of Artificial Sequence Synthetic peptide
YSGFLF

SEQ ID NO: 74 - protein, 7 aa, Artificial Sequence
  Description of Artificial Sequence Synthetic peptide
TGSYYWN

SEQ ID NO: 75 - protein, 16 aa, Artificial Sequence
  Description of Artificial Sequence Synthetic peptide
EINHMGITYYNPSLKG

SEQ ID NO: 76 - protein, 16 aa, Artificial Sequence
  Description of Artificial Sequence Synthetic peptide
EIWHSGPTFYNPSLKS

SEQ ID NO: 77 - protein, 16 aa, Artificial Sequence
  Description of Artificial Sequence Synthetic peptide
EIHGHGFTFYNPSLKS

SEQ ID NO: 78 - protein, 16 aa, Artificial Sequence
  Description of Artificial Sequence Synthetic peptide
EIQSPGYTFYNPSLKS

SEQ ID NO: 79 - protein, 16 aa, Artificial Sequence
  Description of Artificial Sequence Synthetic peptide
TTRYWMSHILAYGMDY

SEQ ID NO: 80 - protein, 13 aa, Artificial Sequence
  Description of Artificial Sequence Synthetic peptide
SGSSSNIGNHYVS

SEQ ID NO: 81 - protein, 7 aa, Artificial Sequence
  Description of Artificial Sequence Synthetic peptide
ANTKRPS

SEQ ID NO: 82 - protein, 10 aa, Artificial Sequence
  Description of Artificial Sequence Synthetic peptide
SSYDGSQSIV

SEQ ID NO: 83 - protein, 126 aa, Artificial Sequence
  Description of Artificial Sequence Synthetic polypeptide
QVQLQESGPGLVKPGETLSLTCTVSGGSISTGSYYWNWIRQAPGKGLEWIGEINHMGITYYNPSLKGRVTISVDTSKNQFSLKLSSVTAEDTAVYYCARTTRYWMSHILAYGMDYWGQGTLVTVSS

SEQ ID NO: 84 - DNA, 378 bp, Artificial Sequence
  Description of Artificial Sequence Synthetic polynucleotide
caggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagatcaatcatatgggcattacctattataatccgagcctgaaaggccgggtgaccattagcgttgatacttcgaaaaaccagtttagcctgaaactgagcagcgtgacggcggaagatacggccgtgtattattgcgcgcgtactactcgttattggatgtctcatattcttgcttatggtatggattattggggccaaggcaccctggtgacggttagctca

SEQ ID NO: 85 - protein, 109 aa, Artificial Sequence
  Description of Artificial Sequence Synthetic polypeptide
DIVLTQPPSVSGAPGQRVTISCSGSSSNIGNHYVSWYQQLPGTAPKLLIYANTKRPSGVPDRFSGSKSGTSASLAITGLQSEDEADYYCSSYDGSQSIVFGGGTKLTVL

SEQ ID NO: 86 - DNA, 327 bp, Artificial Sequence
  Description of Artificial Sequence Synthetic polynucleotide
gatatcgtgctgacccagccgccttcagtgagtggcgcaccaggtcagcgtgtgaccatctcgtgtagcggcagcagcagcaacattggtaatcattatgtgtcttggtaccagcagttgcccgggacggcgccgaaacttctgatttatgctaatactaagcgtccctcaggcgtgccggatcgttttagcggatccaaaagcggcaccagcgcgagccttgcgattacgggcctgcaaagcgaagacgaagcggattattattgctcttcttatgatggttctcagtctattgtgtttggcggcggcacgaagttaaccgtccta

SEQ ID NO: 87 - protein, 126 aa, Artificial Sequence
  Description of Artificial Sequence Synthetic polypeptide
EVQLQESGPGLVKPGETLSLTCTVSGGSISTGSYYWNWIRQAPGKGLEWIGEIQSPGYTFYNPSLKSRVTISVDTSKNQFSLKLSSVTAADTAVYYCARTTRYWMSHILAYGMDYWGQGTLVTVSS

SEQ ID NO: 88 - DNA, 378 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagattcagtctcctggttatactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctca

SEQ ID NO: 89 - DNA, 327 bp, Artificial Sequence; identical to SEQ ID NO: 86
  Description of Artificial Sequence Synthetic polynucleotide

SEQ ID NO: 90 - protein, 126 aa, Artificial Sequence
  Description of Artificial Sequence Synthetic polypeptide
EVQLQESGPGLVKPGETLSLTCTVSGGSISTGSYYWNWIRQAPGKGLEWIGEIWHSGPTFYNPSLKSRVTISVDTSKNQFSLKLSSVTAADTAVYYCARTTRYWMSHILAYGMDYWGQGTLVTVSS

SEQ ID NO: 91 - DNA, 378 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagatttggcattctggtcctactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctca

SEQ ID NO: 92 - DNA, 327 bp, Artificial Sequence; identical to SEQ ID NO: 86
  Description of Artificial Sequence Synthetic polynucleotide

SEQ ID NO: 93 - protein, 126 aa, Artificial Sequence
  Description of Artificial Sequence Synthetic polypeptide
EVQLQESGPGLVKPGETLSLTCTVSGGSISTGSYYWNWIRQAPGKGLEWIGEIHGHGFTFYNPSLKSRVTISVDTSKNQFSLKLSSVTAADTAVYYCARTTRYWMSHILAYGMDYWGQGTLVTVSS

SEQ ID NO: 94 - DNA, 378 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagattcatggtcatggttttactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctca

SEQ ID NO: 95 - DNA, 327 bp, Artificial Sequence; identical to SEQ ID NO: 86
  Description of Artificial Sequence Synthetic polynucleotide

SEQ ID NO: 96 - protein, 456 aa, Artificial Sequence
  Description of Artificial Sequence Synthetic polypeptide
QVQLQESGPGLVKPGETLSLTCTVSGGSISTGSYYWNWIRQAPGKGLEWIGEINHMGITYYNPSLKGRVTISVDTSKNQFSLKLSSVTAEDTAVYYCARTTRYWMSHILAYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 1368 bp, Artificial Sequence
  Description of Artificial Sequence Synthetic polynucleotide
caggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagatcaatcatatgggcattacctattataatccgagcctgaaaggccgggtgaccattagcgttgatacttcgaaaaaccagtttagcctgaaactgagcagcgtgacggcggaagatacggccgtgtattattgcgcgcgtactactcgttattggatgtctcatattcttgcttatggtatggattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 98 - protein, 215 aa, Artificial Sequence
  Description of Artificial Sequence Synthetic polypeptide
DIVLTQPPSVSGAPGQRVTISCSGSSSNIGNHYVSWYQQLPGTAPKLLIYANTKRPSGVPDRFSGSKSGTSASLAITGLQSEDEADYYCSSYDGSQSIVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 99 - DNA, 645 bp, Artificial Sequence
  Description of Artificial Sequence Synthetic polynucleotide
gatatcgtgctgacccagccgccttcagtgagtggcgcaccaggtcagcgtgtgaccatctcgtgtagcggcagcagcagcaacattggtaatcattatgtgtcttggtaccagcagttgcccgggacggcgccgaaacttctgatttatgctaatactaagcgtccctcaggcgtgccggatcgttttagcggatccaaaagcggcaccagcgcgagccttgcgattacgggcctgcaaagcgaagacgaagcggattattattgctcttcttatgatggttctcagtctattgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 100 - protein, 450 aa, Artificial Sequence; identical to SEQ ID NO: 50
  Description of Artificial Sequence Synthetic polypeptide

SEQ ID NO: 101 - DNA, 1350 bp, Artificial Sequence; identical to SEQ ID NO: 51
  Description of Artificial Sequence Synthetic polynucleotide

SEQ ID NO: 102 - DNA, 648 bp, Artificial Sequence; identical to SEQ ID NO: 55
  Description of Artificial Sequence Synthetic polynucleotide

SEQ ID NO: 103 - protein, 456 aa, Artificial Sequence
  Description of Artificial Sequence Synthetic polypeptide
EVQLQESGPGLVKPGETLSLTCTVSGGSISTGSYYWNWIRQAPGKGLEWIGEIQSPGYTFYNPSLKSRVTISVDTSKNQFSLKLSSVTAADTAVYYCARTTRYWMSHILAYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 1368 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagattcagtctcctggttatactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 105 - DNA, 645 bp, Artificial Sequence; identical to SEQ ID NO: 99
  Description of Artificial Sequence Synthetic polynucleotide

SEQ ID NO: 106 - protein, 5 aa, Artificial Sequence
  Description of Artificial Sequence Synthetic peptide
SYAIH

SEQ ID NO: 107 - protein, 17 aa, Artificial Sequence
  Description of Artificial Sequence Synthetic peptide
VISGEGSNTYYADSVKG

SEQ ID NO: 108 - protein, 9 aa, Artificial Sequence
  Description of Artificial Sequence Synthetic peptide
VMIGYGFDY

SEQ ID NO: 109 - protein, 11 aa, Artificial Sequence
  Description of Artificial Sequence Synthetic peptide
RASQSIFNYLN

SEQ ID NO: 110 - protein, 7 aa, Artificial Sequence
  Description of Artificial Sequence Synthetic peptide
DSSTLQS

SEQ ID NO: 111 - protein, 9 aa, Artificial Sequence
  Description of Artificial Sequence Synthetic peptide
LQYSGFLFT

SEQ ID NO: 112 - protein, 118 aa, Artificial Sequence
  Description of Artificial Sequence Synthetic polypeptide
QVQLLESGGGLVQPGGSLRLSCAASGFTFSSYAIHWVRQAPGKGLEWVSVISGEGSNTYYADSVKGRFTISRDNSKNTLYLQMNSLRAEDTAVYYCARVMIGYGFDYWGQGTLVTVSS

SEQ ID NO: 113 - DNA, 354 bp, Artificial Sequence
  Description of Artificial Sequence Synthetic polynucleotide
caggtgcagctgctggaatcaggcggcggactggtgcagcctggcggtagcctgagactgagctgcgctgctagtggcttcaccttctctagctacgctattcactgggtcagacaggcccctggtaaaggcctggagtgggtgtcagtgattagcggcgagggctctaacacctactacgccgatagcgtgaagggccggttcactatctctagggataactctaagaacaccctgtacctgcagatgaatagcctgagagccgaggacaccgccgtctactactgcgctagagtgatgatcggctacggcttcgactactggggtcagggcaccctggtcaccgtgtctagc

SEQ ID NO: 114 - protein, 107 aa, Artificial Sequence
  Description of Artificial Sequence Synthetic polypeptide
DIQMTQSPSSLSASVGDRVTITCRASQSIFNYLNWYQQKPGKAPKLLIYDSSTLQSGVPSRFSGSGSGTDFTLTISSLQPEDFATYYCLQYSGFLFTFGQGTKVEIK

SEQ ID NO: 115 - DNA, 321 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctttaactacctgaactggtatcagcagaagcccggtaaagcccctaagctgctgatctacgactctagcaccctgcagtcaggcgtgccctctaggtttagcggtagcggtagtggcaccgacttcaccctgactatctctagcctgcagcccgaggacttcgctacctactactgcctgcagtatagcggcttcctgttcaccttcggtcagggcactaaggtcgagattaag

SEQ ID NO: 116 - protein, 448 aa, Artificial Sequence
  Description of Artificial Sequence Synthetic polypeptide
QVQLLESGGGLVQPGGSLRLSCAASGFTFSSYAIHWVRQAPGKGLEWVSVISGEGSNTYYADSVKGRFTISRDNSKNTLYLQMNSLRAEDTAVYYCARVMIGYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 1344 bp, Artificial Sequence
  Description of Artificial Sequence Synthetic polynucleotide
caggtgcagctgctggaatcaggcggcggactggtgcagcctggcggtagcctgagactgagctgcgctgctagtggcttcaccttctctagctacgctattcactgggtcagacaggcccctggtaaaggcctggagtgggtgtcagtgattagcggcgagggctctaacacctactacgccgatagcgtgaagggccggttcactatctctagggataactctaagaacaccctgtacctgcagatgaatagcctgagagccgaggacaccgccgtctactactgcgctagagtgatgatcggctacggcttcgactactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 118 - protein, 214 aa, Artificial Sequence
  Description of Artificial Sequence Synthetic polypeptide
DIQMTQSPSSLSASVGDRVTITCRASQSIFNYLNWYQQKPGKAPKLLIYDSSTLQSGVPSRFSGSGSGTDFTLTISSLQPEDFATYYCLQYSGFLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - DNA, 642 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctttaactacctgaactggtatcagcagaagcccggtaaagcccctaagctgctgatctacgactctagcaccctgcagtcaggcgtgccctctaggtttagcggtagcggtagtggcaccgacttcaccctgactatctctagcctgcagcccgaggacttcgctacctactactgcctgcagtatagcggcttcctgttcaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 120 - protein, 5 aa, Artificial Sequence
  Description of Artificial Sequence Synthetic peptide
TFSIS

SEQ ID NO: 121 - protein, 17 aa, Artificial Sequence
  Description of Artificial Sequence Synthetic peptide
GIIPIFGTANYAQKFQG

SEQ ID NO: 122 - protein, 17 aa, Artificial Sequence
  Description of Artificial Sequence Synthetic peptide
TIQSSGENKFYADSVKG

SEQ ID NO: 123 - protein, 11 aa, Artificial Sequence
  Description of Artificial Sequence Synthetic peptide
GGYGGYYYFDY

SEQ ID NO: 124 - protein, 11 aa, Artificial Sequence
  Description of Artificial Sequence Synthetic peptide
RASQSISNRLN

SEQ ID NO: 125 - protein, 7 aa, Artificial Sequence
  Description of Artificial Sequence Synthetic peptide
KGSTLQS

SEQ ID NO: 126 - protein, 9 aa, Artificial Sequence
  Description of Artificial Sequence Synthetic peptide
HQYSGLLFT

SEQ ID NO: 127 - protein, 9 aa, Artificial Sequence
  Description of Artificial Sequence Synthetic peptide
QQHKVWLTT

SEQ ID NO: 128 - protein, 9 aa, Artificial Sequence
  Description of Artificial Sequence Synthetic peptide
QQHYVWSTT

SEQ ID NO: 129 - protein, 9 aa, Artificial Sequence
  Description of Artificial Sequence Synthetic peptide
QQHYQWLTT

SEQ ID NO: 130 - protein, 120 aa, Artificial Sequence
  Description of Artificial Sequence Synthetic polypeptide
QVQLVQSGAEVKKPGSSVKVSCKASGGTFSTFSISWVRQAPGQGLEWMGGIIPIFGTANYAQKFQGRVTITADESTSTAYMELSSLRSEDTAVYYCARGGYGGYYYFDYWGQGTLVTVSS

SEQ ID NO: 131 - DNA, 360 bp, Artificial Sequence
  Description of Artificial Sequence Synthetic polynucleotide
caggtgcagctggtgcagtcaggcgccgaagtgaagaaacccggctctagcgtgaaagtcagctgtaaagctagtggcggcaccttcagcaccttctctattagctgggtcagacaggccccaggtcagggcctggagtggatgggcggaattatccctatcttcggcaccgctaactacgctcagaaatttcagggtagagtgactatcaccgccgacgagtctactagcaccgcctatatggaactgtctagcctgagatcagaggacaccgccgtctactactgcgctaggggcggctacggcggctattactacttcgactactggggtcagggcaccctggtcaccgtgtctagc

SEQ ID NO: 132 - protein, 107 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHKVWLTTFGQGTKVEIK

SEQ ID NO: 133 - DNA, 321 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctctaataggctgaactggtatcagcagaagcccggtaaagcccctaagctgctgatctataagggctctaccctgcagtcaggcgtgccctctaggtttagcggtagcggtagtggcaccgacttcaccctgactatctctagcctgcagcccgaggacttcgctacctactactgtcagcagcacaaagtgtggctgactaccttcggtcagggcactaaggtcgagattaag

SEQ ID NO: 134 - protein, 450 aa, Artificial Sequence
  Description of Artificial Sequence Synthetic polypeptide
QVQLVQSGAEVKKPGSSVKVSCKASGGTFSTFSISWVRQAPGQGLEWMGGIIPIFGTANYAQKFQGRVTITADESTSTAYMELSSLRSEDTAVYYCARGGYGGYYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - DNA, 1350 bp, Artificial Sequence
  Description of Artificial Sequence Synthetic polynucleotide
caggtgcagctggtgcagtcaggcgccgaagtgaagaaacccggctctagcgtgaaagtcagctgtaaagctagtggcggcaccttcagcaccttctctattagctgggtcagacaggccccaggtcagggcctggagtggatgggcggaattatccctatcttcggcaccgctaactacgctcagaaatttcagggtagagtgactatcaccgccgacgagtctactagcaccgcctatatggaactgtctagcctgagatcagaggacaccgccgtctactactgcgctaggggcggctacggcggctattactacttcgactactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 136 - protein, 214 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHKVWLTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - DNA, 642 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctctaataggctgaactggtatcagcagaagcccggtaaagcccctaagctgctgatctataagggctctaccctgcagtcaggcgtgccctctaggtttagcggtagcggtagtggcaccgacttcaccctgactatctctagcctgcagcccgaggacttcgctacctactactgtcagcagcacaaagtgtggctgactaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 138 - protein, 118 aa, Artificial Sequence
  Description of Artificial Sequence Synthetic polypeptide
QVQLLESGGGLVQPGGSLRLSCAASGFTFSSYAIHWVRQAPGKGLEWVSTIQSSGENKFYADSVKGRFTISRDNSKNTLYLQMNSLRAEDTAVYYCARVMIGYGFDYWGQGTLVTVSS

SEQ ID NO: 139 - DNA, 354 bp, Artificial Sequence
  Description of Artificial Sequence Synthetic polynucleotide
caggtgcagctgctggaatcaggcggcggactggtgcagcctggcggtagcctgagactgagctgcgctgctagtggcttcaccttctctagctacgctattcactgggtcagacaggcccctggtaaaggcctggagtgggtcagcactattcagtctagcggcgagaacaagttctacgccgatagcgtgaagggccggttcactatctctagggataactctaagaacaccctgtacctgcagatgaatagcctgagagccgaggacaccgccgtctactactgcgctagagtgatgatcggctacggcttcgactactggggtcagggcaccctggtcaccgtgtctagc

SEQ ID NO: 140 - protein, 107 aa, Artificial Sequence
  Description of Artificial Sequence Synthetic polypeptide
DIQMTQSPSSLSASVGDRVTITCRASQSIFNYLNWYQQKPGKAPKLLIYDSSTLQSGVPSRFSGSGSGTDFTLTISSLQPEDFATYYCHQYSGLLFTFGQGTKVEIK

SEQ ID NO: 141 - DNA, 321 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctttaactacctgaactggtatcagcagaagcccggtaaagcccctaagctgctgatctacgactctagcaccctgcagtcaggcgtgccctctaggtttagcggtagcggtagtggcaccgacttcaccctgactatctctagcctgcagcccgaggacttcgctacctactactgtcaccagtatagcggcctgctgttcaccttcggtcagggcactaaggtcgagattaag

SEQ ID NO: 142 - protein, 448 aa, Artificial Sequence
  Description of Artificial Sequence Synthetic polypeptide
QVQLLESGGGLVQPGGSLRLSCAASGFTFSSYAIHWVRQAPGKGLEWVSTIQSSGENKFYADSVKGRFTISRDNSKNTLYLQMNSLRAEDTAVYYCARVMIGYGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1344 bp, Artificial Sequence
  Description of Artificial Sequence Synthetic polynucleotide
caggtgcagctgctggaatcaggcggcggactggtgcagcctggcggtagcctgagactgagctgcgctgctagtggcttcaccttctctagctacgctattcactgggtcagacaggcccctggtaaaggcctggagtgggtcagcactattcagtctagcggcgagaacaagttctacgccgatagcgtgaagggccggttcactatctctagggataactctaagaacaccctgtacctgcagatgaatagcctgagagccgaggacaccgccgtctactactgcgctagagtgatgatcggctacggcttcgactactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 144 - protein, 214 aa, Artificial Sequence
  Description of Artificial Sequence Synthetic polypeptide
DIQMTQSPSSLSASVGDRVTITCRASQSIFNYLNWYQQKPGKAPKLLIYDSSTLQSGVPSRFSGSGSGTDFTLTISSLQPEDFATYYCHQYSGLLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - DNA, 642 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctttaactacctgaactggtatcagcagaagcccggtaaagcccctaagctgctgatctacgactctagcaccctgcagtcaggcgtgccctctaggtttagcggtagcggtagtggcaccgacttcaccctgactatctctagcctgcagcccgaggacttcgctacctactactgtcaccagtatagcggcctgctgttcaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 146 - DNA, 360 bp, Artificial Sequence; identical to SEQ ID NO: 131
  Description of Artificial Sequence Synthetic polynucleotide

SEQ ID NO: 147 - protein, 107 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHYVWSTTFGQGTKVEIK

SEQ ID NO: 148 - DNA, 321 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctctaataggctgaactggtatcagcagaagcccggtaaagcccctaagctgctgatctataagggctctaccctgcagtcaggcgtgccctctaggtttagcggtagcggtagtggcaccgacttcaccctgactatctctagcctgcagcccgaggacttcgctacctactactgtcagcagcactacgtgtggtctactaccttcggtcagggcactaaggtcgagattaag

SEQ ID NO: 149 - DNA, 1350 bp, Artificial Sequence; identical to SEQ ID NO: 135
  Description of Artificial Sequence Synthetic polynucleotide

SEQ ID NO: 150 - protein, 214 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHYVWSTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - DNA, 642 bp, Artificial Sequence
  Description of Artificial Sequence Synthetic polynucleotide
gatattcagatgactcagtcacctagtagcctgagcgctagtgtgggcgatagagtgactatcacctgtagagcctctcagtctatctctaataggctgaactggtatcagcagaagcccggtaaagcccctaagctgctgatctataagggctctaccctgcagtcaggcgtgccctctaggtttagcggtagcggtagtggcaccgacttcaccctgactatctctagcctgcagcccgaggacttcgctacctactactgtcagcagcactacgtgtggtctactaccttcggtcagggcactaaggtcgagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 152 - DNA, 360 bp, Artificial Sequence
  Description of Artificial Sequence Synthetic polynucleotide
caggtgcaattggtgcagagcggtgccgaagtgaaaaaaccgggcagcagcgtgaaagttagctgcaaagcatccggagggacgttttctactttctctatctcttgggtgcgccaggccccgggccagggcctcgagtggatgggcggtatcatcccgatcttcggcactgcgaactacgcccagaaatttcagggccgggtgaccattaccgccgatgaaagcaccagcaccgcctatatggaactgagcagcctgcgcagcgaagatacggccgtgtattattgcgcgcgtggtggttacggtggttactactacttcgattactggggccaaggcaccctggtgactgttagctca

SEQ ID NO: 153 - protein, 107 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHYQWLTTFGQGTKVEIK

SEQ ID NO: 154 - DNA, 321 bp, Artificial Sequence
  Description of Artificial Sequence Synthetic polynucleotide
gatatccagatgacccagagcccgagcagcctgagcgccagcgtgggcgatcgcgtgaccattacctgcagagccagccagtctatttctaaccgtctgaactggtaccagcagaaaccgggcaaagcgccgaaactattaatctacaaaggttctactctgcaaagcggcgtgccgagccgctttagcggcagcggatccggcaccgatttcaccctgaccattagctctctgcaaccggaagactttgcgacctattattgccagcagcattaccagtggctgactacctttggccagggcacgaaagttgaaattaaa

SEQ ID NO: 155 - DNA, 1350 bp, Artificial Sequence
  Description of Artificial Sequence Synthetic polynucleotide
caggtgcaattggtgcagagcggtgccgaagtgaaaaaaccgggcagcagcgtgaaagttagctgcaaagcatccggagggacgttttctactttctctatctcttgggtgcgccaggccccgggccagggcctcgagtggatgggcggtatcatcccgatcttcggcactgcgaactacgcccagaaatttcagggccgggtgaccattaccgccgatgaaagcaccagcaccgcctatatggaactgagcagcctgcgcagcgaagatacggccgtgtattattgcgcgcgtggtggttacggtggttactactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 156 - protein, 214 aa, Artificial Sequence
  Description of Artificial Sequence Synthetic polypeptide
DIQMTQSPSSLSASVGDRVTITCRASQSISNRLNWYQQKPGKAPKLLIYKGSTLQSGVPSRFSGSGSGTDFTLTISSLQPEDFATYYCQQHYQWLTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - DNA, 642 bp, Artificial Sequence
  Description of Artificial Sequence Synthetic polynucleotide
gatatccagatgacccagagcccgagcagcctgagcgccagcgtgggcgatcgcgtgaccattacctgcagagccagccagtctatttctaaccgtctgaactggtaccagcagaaaccgggcaaagcgccgaaactattaatctacaaaggttctactctgcaaagcggcgtgccgagccgctttagcggcagcggatccggcaccgatttcaccctgaccattagctctctgcaaccggaagactttgcgacctattattgccagcagcattaccagtggctgactacctttggccagggcacgaaagttgaaattaaacgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgt

SEQ ID NO: 158 - protein, 449 aa, Artificial Sequence
  Description of Artificial Sequence Synthetic polypeptide
QVQLVQSGAEVKKPGSSVKVSCKASGGTFSSYAISWVRQAPGQGLEWMGNIIPITGQTYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 1347 bp, Artificial Sequence
  Description of Artificial Sequence Synthetic polynucleotide
caggtgcagctggtgcagtcaggcgccgaagtgaagaaacccggctctagcgtgaaagtcagctgtaaagctagtggcggcaccttctctagctacgctattagctgggtcagacaggccccaggtcagggcctggagtggatgggcaatattatccctatcaccggtcagacctactacgctcagaaatttcagggtagagtgactatcaccgccgacgagtctactagcaccgcctatatggaactgtctagcctgagatcagaggacaccgccgtctactactgcgctagagccgcctatcaccccctggtgttcgataactggggtcagggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaagctgctggcggcccttctgtgttcctgttccctccaaagcccaaggacaccctgatgatctcccggacccctgaagtgacctgcgtggtggtggacgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctgcctgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 160 - protein, 216 aa, Artificial Sequence
  Description of Artificial Sequence Synthetic polypeptide
QSVLTQPPSASGTPGQRVTISCSGSSSNIGNHYVNWYQQLPGTAPKLLIYRNNHRPSGVPDRFSGSKSGTSASLAISGLQSEDEADYYCQSWDYSGFSTVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 161 - DNA, 648 bp, Artificial Sequence
  Description of Artificial Sequence Synthetic polynucleotide
cagtcagtcctgactcagccccctagcgctagtggcacccctggtcagagagtgactattagctgtagcggctctagctctaatatcggtaatcactacgtgaactggtatcagcagctgcccggcaccgcccctaagctgctgatctatagaaacaatcaccggcctagcggcgtgcccgataggtttagcggatctaagtcagggactagcgctagtctggctattagcggcctgcagtcagaggacgaggccgactactactgtcagtcctgggactatagcggctttagcaccgtgttcggcggaggcactaagctgaccgtgctgggtcagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 162 - protein, 9 aa, Artificial Sequence
  Description of Artificial Sequence Synthetic peptide
GGSISTGSY

SEQ ID NO: 163 - protein, 5 aa, Artificial Sequence
  Description of Artificial Sequence Synthetic peptide
QSPGY

SEQ ID NO: 164 - protein, 16 aa, Artificial Sequence
  Description of Artificial Sequence Synthetic peptide
TTRYWMSHILAYGMDY

SEQ ID NO: 165 - protein, 9 aa, Artificial Sequence
  Description of Artificial Sequence Synthetic peptide
SSSNIGNHY

SEQ ID NO: 166 - protein, 3 aa, Artificial Sequence
  Description of Artificial Sequence Synthetic peptide
ANT

SEQ ID NO: 167 - protein, 7 aa, Artificial Sequence
  Description of Artificial Sequence Synthetic peptide
YDGSQSI

SEQ ID NO: 168 - protein, 456 aa, Artificial Sequence
  Description of Artificial Sequence Synthetic polypeptide
EVQLQESGPGLVKPGETLSLTCTVSGGSISTGSYYWNWIRQAPGKGLEWIGEIWHSGPTFYNPSLKSRVTISVDTSKNQFSLKLSSVTAADTAVYYCARTTRYWMSHILAYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - DNA, 1368 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagatttggcattctggtcctactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 170 - protein, 215 aa, Artificial Sequence; identical to SEQ ID NO: 98
  Description of Artificial Sequence Synthetic polypeptide

SEQ ID NO: 171 - DNA, 645 bp, Artificial Sequence; identical to SEQ ID NO: 99
  Description of Artificial Sequence Synthetic polynucleotide

SEQ ID NO: 172 - protein, 456 aa, Artificial Sequence
  Description of Artificial Sequence Synthetic polypeptide
EVQLQESGPGLVKPGETLSLTCTVSGGSISTGSYYWNWIRQAPGKGLEWIGEIHGHGFTFYNPSLKSRVTISVDTSKNQFSLKLSSVTAADTAVYYCARTTRYWMSHILAYGMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - DNA, 1368 bp, Artificial Sequence
  Description of Artificial Sequence Synthetic polynucleotide
gaggtgcaattgcaagaaagtggtccgggcctggtgaaaccgggcgaaaccctgagcctgacctgcaccgtttccggaggtagcatttctactggttcttattattggaattggattcgccaggcccctgggaagggtctcgagtggattggcgagattcatggtcatggttttactttttataatccttctcttaagtctcgggtgaccattagcgttgatacttcgaaaaaccagtttagcctgaaactgagcagcgtgacggcggcggatacggccgtgtattattgcgcgcgtactactcgttattggatgtctcatattcttgcttatggtatggattattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 174 - protein, 215 aa, Artificial Sequence; identical to SEQ ID NO: 98
  Description of Artificial Sequence Synthetic polypeptide

SEQ ID NO: 175 - DNA, 645 bp, Artificial Sequence; identical to SEQ ID NO: 99
  Description of Artificial Sequence Synthetic polynucleotide

SEQ ID NO: 176 - protein, 449 aa, Artificial Sequence
  Description of Artificial Sequence Synthetic polypeptide
EVQLVQSGAEVKKPGSSVKVSCKASGGTFNSYAISWVRQAPGQGLEWMGNIIPHYGFAYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - DNA, 1347 bp, Artificial Sequence
  Description of Artificial Sequence Synthetic polynucleotide
gaggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcaatattattcctcattatggttttgcttattatgctcagaagtttcagggtcgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 178 - protein, 216 aa, Artificial Sequence; identical to SEQ ID NO: 45
  Description of Artificial Sequence Synthetic polypeptide

SEQ ID NO: 179 - DNA, 648 bp, Artificial Sequence; identical to SEQ ID NO: 58
  Description of Artificial Sequence Synthetic polynucleotide

SEQ ID NO: 180 - protein, 449 aa, Artificial Sequence
  Description of Artificial Sequence Synthetic polypeptide
EVQLVQSGAEVKKPGSSVKVSCKASGGTFNSYAISWVRQAPGQGLEWMGNIIPYSGFAYYAQKFQGRVTITADESTSTAYMELSSLRSEDTAVYYCARAAYHPLVFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 181 - DNA, 1347 bp, Artificial Sequence
  Description of Artificial Sequence Synthetic polynucleotide
gaggtgcaattggttcagtctggcgcggaagtgaaaaaaccgggcagcagcgtgaaagtgagctgcaaagcctccggaggcacttttaattcttatgctatttcttgggtgcgccaagcccctgggcagggtctcgagtggatgggcaatattattccttattctggttttgcttattatgctcagaagtttcagggtcgggtgaccattaccgcggatgaaagcaccagcaccgcgtatatggaactgagcagcctgcgtagcgaagatacggccgtgtattattgcgcgcgtgctgcttatcatcctcttgtttttgataattggggccaaggcaccctggtgacggttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 182 - protein, 216 aa, Artificial Sequence; identical to SEQ ID NO: 45
  Description of Artificial Sequence Synthetic polypeptide

SEQ ID NO: 183 - DNA, 648 bp, Artificial Sequence; identical to SEQ ID NO: 58
  Description of Artificial Sequence Synthetic polynucleotide

SEQ ID NO: 184 - protein, 9 aa, Artificial Sequence
  Description of Artificial Sequence Synthetic peptide
GGSISTGSY

SEQ ID NO: 185 - protein, 5 aa, Artificial Sequence
  Description of Artificial Sequence Synthetic peptide
NHMGI

SEQ ID NO: 186 - protein, 16 aa, Artificial Sequence
  Description of Artificial Sequence Synthetic peptide
TTRYWMSHILAYGMDY

SEQ ID NO: 187 - protein, 9 aa, Artificial Sequence
  Description of Artificial Sequence Synthetic peptide
SSSNIGNHY

SEQ ID NO: 188 - protein, 3 aa, Artificial Sequence
  Description of Artificial Sequence Synthetic peptide
ANT

SEQ ID NO: 189 - protein, 7 aa, Artificial Sequence
  Description of Artificial Sequence Synthetic peptide
YDGSQSI

SEQ ID NO: 190 - protein, 9 aa, Artificial Sequence
  Description of Artificial Sequence Synthetic peptide
GGSISTGSY

SEQ ID NO: 191 - protein, 5 aa, Artificial Sequence
  Description of Artificial Sequence Synthetic peptide
WHSGP

SEQ ID NO: 192 - protein, 16 aa, Artificial Sequence
  Description of Artificial Sequence Synthetic peptide
TTRYWMSHILAYGMDY

SEQ ID NO: 193 - protein, 9 aa, Artificial Sequence
  Description of Artificial Sequence Synthetic peptide
SSSNIGNHY

SEQ ID NO: 194 - protein, 3 aa, Artificial Sequence
  Description of Artificial Sequence Synthetic peptide
ANT

SEQ ID NO: 195 - protein, 7 aa, Artificial Sequence
  Description of Artificial Sequence Synthetic peptide
YDGSQSI

SEQ ID NO: 196 - protein, 16 aa, Artificial Sequence
  Description of Artificial Sequence Synthetic peptide
EIHGHGFTFYNPSLKS

SEQ ID NO: 197 - protein, 9 aa, Artificial Sequence
  Description of Artificial Sequence Synthetic peptide
GGSISTGSY

SEQ ID NO: 198 - protein, 5 aa, Artificial Sequence
  Description of Artificial Sequence Synthetic peptide
HGHGF

SEQ ID NO: 199 - protein, 16 aa, Artificial Sequence
  Description of Artificial Sequence Synthetic peptide
TTRYWMSHILAYGMDY

SEQ ID NO: 200 - protein, 9 aa, Artificial Sequence
  Description of Artificial Sequence Synthetic peptide
SSSNIGNHY

SEQ ID NO: 201 - protein, 3 aa, Artificial Sequence
  Description of Artificial Sequence Synthetic peptide
ANT

SEQ ID NO: 202 - protein, 7 aa, Artificial Sequence
  Description of Artificial Sequence Synthetic peptide
YDGSQSI

SEQ ID NO: 203 - protein, 7 aa, Artificial Sequence
  Description of Artificial Sequence Synthetic peptide
GGTFNSY

SEQ ID NO: 204 - protein, 6 aa, Artificial Sequence
  Description of Artificial Sequence Synthetic peptide
IPIYGT

SEQ ID NO: 205 - protein, 10 aa, Artificial Sequence
  Description of Artificial Sequence Synthetic peptide
AAYHPLVFDN

SEQ ID NO: 206 - protein, 9 aa, Artificial Sequence
  Description of Artificial Sequence Synthetic peptide
SSSNIGNHY

SEQ ID NO: 207 - protein, 3 aa, Artificial Sequence
  Description of Artificial Sequence Synthetic peptide
RNN

SEQ ID NO: 208 - protein, 8 aa, Artificial Sequence
  Description of Artificial Sequence Synthetic peptide
WDYSGFST

SEQ ID NO: 209 - protein, 7 aa, Artificial Sequence
  Description of Artificial Sequence Synthetic peptide
GGTFNSY

SEQ ID NO: 210 - protein, 7 aa, Artificial Sequence
  Description of Artificial Sequence Synthetic peptide
NPFYIGE

SEQ ID NO: 211 - protein, 10 aa, Artificial Sequence
  Description of Artificial Sequence Synthetic peptide
AAYHPLVFDN

SEQ ID NO: 212 - protein, 9 aa, Artificial Sequence
  Description of Artificial Sequence Synthetic peptide
SSSNIGNHY

SEQ ID NO: 213 - protein, 3 aa, Artificial Sequence
  Description of Artificial Sequence Synthetic peptide
RNN

SEQ ID NO: 214 - protein, 8 aa, Artificial Sequence
  Description of Artificial Sequence Synthetic peptide
WDYSGFST

SEQ ID NO: 215 - protein, 7 aa, Artificial Sequence
  Description of Artificial Sequence Synthetic peptide
GGTFNSY

SEQ ID NO: 216 - protein, 6 aa, Artificial Sequence
  Description of Artificial Sequence Synthetic peptide
IPHYGF

SEQ ID NO: 217 - protein, 10 aa, Artificial Sequence
  Description of Artificial Sequence Synthetic peptide
AAYHPLVFDN

SEQ ID NO: 218 - protein, 9 aa, Artificial Sequence
  Description of Artificial Sequence Synthetic peptide
SSSNIGNHY

SEQ ID NO: 219 - protein, 3 aa, Artificial Sequence
  Description of Artificial Sequence Synthetic peptide
RNN

SEQ ID NO: 220 - protein, 8 aa, Artificial Sequence
  Description of Artificial Sequence Synthetic peptide
WDYSGFST

SEQ ID NO: 221 - protein, 7 aa, Artificial Sequence
  Description of Artificial Sequence Synthetic peptide
GGTFNSY

SEQ ID NO: 222 - protein, 6 aa, Artificial Sequence
  Description of Artificial Sequence Synthetic peptide
IPYSGF

SEQ ID NO: 223 - protein, 10 aa, Artificial Sequence
  Description of Artificial Sequence Synthetic peptide
AAYHPLVFDN

SEQ ID NO: 224 - protein, 9 aa, Artificial Sequence
  Description of Artificial Sequence Synthetic peptide
SSSNIGNHY

SEQ ID NO: 225 - protein, 3 aa, Artificial Sequence
  Description of Artificial Sequence Synthetic peptide
RNN

SEQ ID NO: 226 - protein, 8 aa, Artificial Sequence
  Description of Artificial Sequence Synthetic peptide
WDYSGFST

SEQ ID NO: 227 - protein, 7 aa, Artificial Sequence
  Description of Artificial Sequence Synthetic peptide
GGTFNSY

SEQ ID NO: 228 - protein, 7 aa, Artificial Sequence
  Description of Artificial Sequence Synthetic peptide
NPFYIGE

SEQ ID NO: 229 - protein, 10 aa, Artificial Sequence
  Description of Artificial Sequence Synthetic peptide
AAYHPLVFDN

SEQ ID NO: 230 - protein, 9 aa, Artificial Sequence
  Description of Artificial Sequence Synthetic peptide
SSSNIGNHY

SEQ ID NO: 231 - protein, 3 aa, Artificial Sequence
  Description of Artificial Sequence Synthetic peptide
RNN

SEQ ID NO: 232 - protein, 8 aa, Artificial Sequence
  Description of Artificial Sequence Synthetic peptide
WDYSGFST

SEQ ID NO: 233 - protein, 7 aa, Artificial Sequence
  Description of Artificial Sequence Synthetic peptide
GGTFNSY

SEQ ID NO: 234 - protein, 6 aa, Artificial Sequence
  Description of Artificial Sequence Synthetic peptide
IPMTGQ

SEQ ID NO: 235 - protein, 10 aa, Artificial Sequence
  Description of Artificial Sequence Synthetic peptide
AAYHPLVFDN

SEQ ID NO: 236 - protein, 9 aa, Artificial Sequence
  Description of Artificial Sequence Synthetic peptide
SSSNIGNHY

SEQ ID NO: 237 - protein, 3 aa, Artificial Sequence
  Description of Artificial Sequence Synthetic peptide
RNN

SEQ ID NO: 238 - protein, 8 aa, Artificial Sequence
  Description of Artificial Sequence Synthetic peptide
WDYSGFST

SEQ ID NO: 239 - protein, 17 aa, Artificial Sequence
  Description of Artificial Sequence Synthetic peptide
NIIPITGQTYYAQKFQG

SEQ ID NO: 240 - protein, 7 aa, Artificial Sequence
  Description of Artificial Sequence Synthetic peptide
GGTFSTF

SEQ ID NO: 241 - protein, 6 aa, Artificial Sequence
  Description of Artificial Sequence Synthetic peptide
IPIFGT

SEQ ID NO: 242 - protein, 11 aa, Artificial Sequence
  Description of Artificial Sequence Synthetic peptide
GGYGGYYYFDY

SEQ ID NO: 243 - protein, 7 aa, Artificial Sequence
  Description of Artificial Sequence Synthetic peptide
SQSISNR

SEQ ID NO: 244 - protein, 3 aa, Artificial Sequence
  Description of Artificial Sequence Synthetic peptide
KGS

SEQ ID NO: 245 - protein, 6 aa, Artificial Sequence
  Description of Artificial Sequence Synthetic peptide
HKVWLT

SEQ ID NO: 246 - protein, 7 aa, Artificial Sequence
  Description of Artificial Sequence Synthetic peptide
GGTFSTF

SEQ ID NO: 247 - protein, 6 aa, Artificial Sequence
  Description of Artificial Sequence Synthetic peptide
IPIFGT

SEQ ID NO: 248 - protein, 11 aa, Artificial Sequence
  Description of Artificial Sequence Synthetic peptide
GGYGGYYYFDY

SEQ ID NO: 249 - protein, 7 aa, Artificial Sequence
  Description of Artificial Sequence Synthetic peptide
SQSISNR

SEQ ID NO: 250 - protein, 3 aa, Artificial Sequence
  Description of Artificial Sequence Synthetic peptide
KGS

SEQ ID NO: 251 - protein, 6 aa, Artificial Sequence
  Description of Artificial Sequence Synthetic peptide
HYVWST

SEQ ID NO: 252 - protein, 7 aa, Artificial Sequence
  Description of Artificial Sequence Synthetic peptide
GGTFSTF

SEQ ID NO: 253 - protein, 6 aa, Artificial Sequence
  Description of Artificial Sequence Synthetic peptide
IPIFGT

SEQ ID NO: 254 - protein, 11 aa, Artificial Sequence
  Description of Artificial Sequence Synthetic peptide
GGYGGYYYFDY

SEQ ID NO: 255 - protein, 7 aa, Artificial Sequence
  Description of Artificial Sequence Synthetic peptide
SQSISNR

SEQ ID NO: 256 - protein, 3 aa, Artificial Sequence
  Description of Artificial Sequence Synthetic peptide
KGS

SEQ ID NO: 257 - protein, 6 aa, Artificial Sequence
  Description of Artificial Sequence Synthetic peptide
HYQWLT

SEQ ID NO: 258 - protein, 7 aa, Artificial Sequence
  Description of Artificial Sequence Synthetic peptide
GFTFSSY

SEQ ID NO: 259 - protein, 6 aa, Artificial Sequence
  Description of Artificial Sequence Synthetic peptide
QSSGEN

SEQ ID NO: 260 - protein, 9 aa, Artificial Sequence
  Description of Artificial Sequence Synthetic peptide
VMIGYGFDY

SEQ ID NO: 261 - protein, 7 aa, Artificial Sequence
  Description of Artificial Sequence Synthetic peptide
SQSIFNY

SEQ ID NO: 262 - protein, 3 aa, Artificial Sequence
  Description of Artificial Sequence Synthetic peptide
DSS

SEQ ID NO: 263 - protein, 6 aa, Artificial Sequence
  Description of Artificial Sequence Synthetic peptide
YSGLLF

(19) United States
(12) Patent Application Publication
(10) Pub. No.: US 2024/0360210 A1
(43) Pub. Date: Oct. 31, 2024

(54) USE OF AN IL-18 ANTAGONIST FOR TREATING AND/OR PREVENTION OF ATOPIC DERMATITIS OR A RELATED CONDITION

(71) Applicant: NOVARTIS AG, Basel (CH)

(72) Inventors: Enrico FERRERO, Basel (CH); Tobias JUNT, Liestal (CH); Frank KOLBINGER, Neuenburg (DE); Jiri KOVARIK, Zürich (CH); Christian LOESCHE, Hegenheim (FR)

(21) Appl. No.: 18/034,326

(22) PCT Filed: Oct. 29, 2021
(86) PCT No.: PCT/IB2021/060006

§ 371 Date: Apr. 27, 2023

Related U.S. Application Data
(60) Provisional application No. 63/107,340, filed on Oct. 29, 2020.

(51) Int. Cl. C07K 16/24; A61K 39/00; A61K 45/06; A61P 17/00

(52) U.S. Cl. CPC C07K 16/244; A61K 45/06; A61P 17/00; A61K 2039/505

(57) ABSTRACT

The present invention relates to the treatment and/or prevention of atopic dermatitis or a related condition. More specifically, the invention relates to the administration of an IL-18 antagonist, e.g., an anti-IL-18 antibody or a fragment thereof, to treat or prevent atopic dermatitis or a related condition in a subject in need thereof.

### SEQUENCE LISTING

The instant application contains a Sequence Listing which has been submitted electronically in ASCII format and is hereby incorporated by reference in its entirety. Said ASCII copy, created on Oct. 21, 2020, is named PAT058945_Sequence_Listing_ST25.txt and is 220 KB in size.

### FIELD

The present invention relates to the treatment and/or prevention of atopic dermatitis or a related condition. More specifically, the invention relates to the administration of an IL-18 antagonist, e.g., an anti-IL-18 antibody or a fragment thereof, to treat or prevent atopic dermatitis in a subject in need thereof.

### BACKGROUND

Atopic dermatitis (AD) is a chronic/relapsing inflammatory skin disease characterized by symptoms including intense pruritus (e.g., severe itch) and by scaly and dry eczematous lesions. Severe disease can be extremely disabling due to major psychological problems, significant sleep loss, and impaired quality of life, leading to high socioeconomic costs. The pathophysiology of AD is influenced by a complex interplay between Immunoglobulin E (IgE)-mediated sensitization, the immune system, and environmental factors. The primary skin defect may be an immunological disturbance that causes IgE-mediated sensitization, with epithelial-barrier dysfunction that is the consequence of both genetic mutations and local inflammation. AD often begins in childhood before age 5 and may persist into adulthood.

Typical treatments for AD include topical lotions and moisturizers, topical corticosteroid ointments, creams or injections. Most treatment options, however, offer only temporary, incomplete, symptom relief. Moreover, many patients with moderate-to-severe AD become resistant to treatment by topical corticosteroids or by calcineurin inhibitors. Thus, a need exists in the art for novel targeted therapies for the treatment and/or prevention of AD.

Interleukin-18 (IL-18) is a pro-inflammatory cytokine associated with the induction of Th1 responses, enhanced type I macrophage activation and NK/CD8+ T cell cytotoxicity (Okamura et al. (1995) Nature; 378:88-91; Yoshimoto et al. (1998) J Immunol; 161(7):3400-7; Arend et al. (2008) Immunol Rev.; 223:20-38). IL-18 was originally described in 1989 as interferon-gamma inducing factor (IGIF). IL-18 is related to the IL-1 family and is structurally related to IL-1β (Okamura et al. (1995) Nature; 378:88-91). IL-18 is primarily produced by macrophages and T cells as a precursor protein (pro-IL-18) and secreted as an active protein following cleavage by caspase-1 (Dinarello C A et al (1999) J Allergy Clin Immunol; 103:11-24). In addition to macrophages and T cells, pro-IL-18 is produced by a wide variety of other cells, including keratinocytes, intestinal epithelial cells, and osteoblasts.

In normal physiology IL-18, in synergy with IL-12, is associated with induction of cell-mediated immunity following infection with microbial products such as lipopolysaccharide (LPS) (Sareneva T et al. (2000) J Immunol; 165(4):1933-8). After stimulation with IL-18, natural killer (NK) cells and T cells release the cytokine interferon gamma (IFN-γ) which plays an important role in activating macrophages and other cells. IL-18 has also various functions in addition to an ability to induce interferon gamma. These biological properties include activation of NF-κB, Fas ligand expression, the induction of both CC and CXC chemokines, and increased production of competent human immuno-deficiency virus. Due to the ability of IL-18 to induce IFN-γ production in T cells and macrophages, it plays an important role in Th1-type immune responses and participates in both innate and acquired immunity. IL-18 is a pleiotropic cytokine involved in T cell, NK cell, mast cell, basophil and macrophage activation and survival and has the property to facilitate Th2 responses dependent on surrounding cytokine milieu (Kaplanski (2018) Immunol Rev 281: 138-153; Nakanishi (2018) Front Immunol 9: 763).

Apart from its physiological role, IL-18 has been shown to mediate a variety of autoimmune, such as Crohn's disease, psoriasis, rheumatoid arthritis, multiple sclerosis and cardiovascular diseases (Braddock et al. (2004) Expert Opin Biol Ther; 4(6):847-860), and inflammatory diseases. It has been demonstrated that IL-18 expression is up-regulated in several autoimmune diseases, such as chronic obstructive pulmonary disease (COPD) (Imaoka et al. (2008) Eur Respir; J31:287-297), idiopathic pulmonary fibrosis (IPF) (Kitasato et al. (2004) Am J Resp Cell Mol Biol; 31:619-625), macrophage activation syndrome (MAS) (Dinarello and Kaplanski (2005) Expert Rev Clin Immunol; 1(4): 619-632), adult onset Still's disease (AOSD) (Arlet J B et al. (2006) Ann Rheum Dis 65(12):1596-601) and systemic juvenile idiopathic arthritis (SJIA) (Akashi et al. (1994) Br J Haematol; 87(2):243-50). Serum IL-18 levels have been shown to be increased in AD patients, and to correlate with disease severity (Thijs et al. (2015) Clin Exp Allergy 45: 698-701, Zedan et al. (2015) J Clin Diagn Res 9: WC01-05, Gohar et al. (2017) Egypt J Immunol 24: 9-22). IL-18 was shown to be overexpressed in the epidermis of pediatric AD participants and associated with AD disease activity (McAleer et al. (2019) Br J Dermatol 180: 586-596, Hulshof et al. (2019) Br J Dermatol 180: 621-630).

In preclinical models such as K14/IL-18 transgenic mice which overexpress IL-18 in keratinocytes in the skin, IL-18 contributes to the development of AD-like inflammatory lesions independently of IgE/STAT6 (Konishi et al. (2002) Proc Natl Acad Sci USA 99: 11340-11345). Similarly, keratinocyte-specific Casp1 transgenic mice (K14Casp1Tg), which display high levels of mature IL-18, develop AD like itchy skin lesions with age (Tsutsui et al. (2011) Curr Probl Dermatol 41: 93-103; Yamanaka et al. (2000) J Immunol 165: 997-1003) and blockade of IL-18 prevented S. aureus induced AD-like disease model in mice (Terada et al. (2006) Proc Natl Acad Sci USA 103: 8816-8821). In vivo administration of IL-18 causes Th2 differentiation and increases IgE production in a CD4+ T cell-, IL-4- and STAT6-dependent fashion in mice (Yoshimoto et al. (2000) Nat Immunol 1: 132-137; Hoshino et al. (2000) Eur J Immunol 30: 1998-2006).

Antibodies that antagonize IL-18 were disclosed (e.g. in WO 2014/037899), and are fully human, Fc-silenced (IgG1-LALA), high affinity monoclonal antibodies that selectively bind to IL-18 and inhibit IL-18 activity.

The present invention relates to the use of an IL-18 antagonist, e.g., anti-IL-18 antibody or fragment thereof, in the treatment or prevention of atopic dermatitis or a related condition.

### SUMMARY

The present invention relates to the findings that an IL-18 antagonist, e.g., an anti-IL-18 antibody or a fragment thereof, can be used for the treatment and/or prevention of atopic dermatitis (AD) or a related condition. The present invention identifies AD as an indication for an IL-18 antagonist, e.g., anti-IL-18 antibody or a fragment thereof. Recent publications associated IL-18 levels with AD disease activity (Thijs et al. (2015) Clin Exp Allergy 45: 698-701, Zedan et al. (2015) J Clin Diagn Res 9: WC01-05, Gohar et al. (2017) Egypt J Immunol 24: 9-22; McAleer et al. (2019) Br J Dermatol 180: 586-596, Hulshof et al. (2019) Br J Dermatol 180: 621-630). In preclinical mouse models it has been demonstrated that IL-18 contributes to the development of AD-like inflammatory lesions (Konishi et al. (2002) Proc Natl Acad Sci USA 99: 11340-11345; Tsutsui et al. (2011) Curr Probl Dermatol 41: 93-103; Yamanaka et al. (2000) J Immunol 165: 997-1003). However, even though IL-18 represents a potential therapeutic target for AD, there is no evidence to date that AD could be potentially treatable by antagonizing IL-18. Furthermore, AD is considered to be driven by IL-13 producing T helper 2 cells, while IL-18 was described to be largely stimulating T helper 1 cells and NK cells. The effects of an IL-18 antagonist, such as anti-IL-18 antibody or a fragment thereof, for AD as exemplified in the working examples herein, was thus not predictable.

In one aspect, provided herein are IL-18 antagonists, e.g., anti-IL-18 antibody or a fragment thereof, for use in the treatment and/or prevention of AD or a related condition.

In another aspect, provided herein are methods of treatment and/or prevention of AD or a related condition in a subject in need thereof, comprising administering an IL-18 antagonist, e.g., anti-IL-18 antibody or a fragment thereof.

In a further aspect, provided herein are uses of an IL-18 antagonist, e.g., anti-IL-18 antibody or a fragment thereof, in the treatment and/or prevention of AD or a related condition.

In a further aspect, provided herein are uses of an I1-18 antagonist, e.g., anti-IL-18 antibody or a fragment thereof, for the manufacture of a medicament for treatment and/or prevention of AD or a related condition.

Other aspects and embodiments provided herein will become apparent from a review of the ensuing detailed description.

### BRIEF DESCRIPTION OF THE DRAWINGS

FIG. 1. IL-18 levels in ex vivo cultured skin. Non-lesional and lesional skin biopsies from atopic dermatitis patients or skin biopsies from healthy volunteers were cultured for 24 h. Total IL-18 supernatant levels were analyzed with MSD Assay (Meso Scale Discovery) (A). Mature IL-18 levels in the culture supernatant were analyzed with an IL-18 ELISA (B and C). Measured IL-18 concentrations were normalized by the weight of each biopsy piece. Values below level of detection (not enough sample volume was left, samples had to be diluted) are indicated by open symbols.

FIG. 2. Soluble CD40 levels. Relative soluble CD40 levels (per mg biopsy) in supernatants of ex vivo untreated cultured skin of healthy people and atopic dermatitis patients (A and B). NL=non-lesional, L=lesional. Relative soluble CD40 levels (per mg biopsy) in supernatants of ex vivo cultured skin of atopic dermatitis patients in absence (untreated) or presence of CMK389 (C).

FIG. 3. IL-24 levels. Relative IL-24 levels (per mg biopsy) in supernatants of ex vivo untreated cultured skin of healthy people and atopic dermatitis patients (A and B). NL=non-lesional, L=lesional. Relative IL-24 levels (per mg biopsy) in supernatants of ex vivo cultured skin of atopic dermatitis patients in absence (untreated) or presence of CMK389 (C).

FIG. 4. TARC/CCL17 levels. Relative TARC/CCL17 levels (per mg biopsy) in supernatants of ex vivo untreated cultured skin of healthy people and atopic dermatitis patients (A and B). NL=non-lesional, L=lesional. Relative TARC/CCL17 levels (per mg biopsy) in supernatants of ex vivo cultured skin of atopic dermatitis patients in absence (untreated) or presence of CMK389 (C).

FIG. 5. IL-22 levels. Relative IL-22 levels (per mg biopsy) in supernatants of ex vivo untreated cultured skin of healthy people and atopic dermatitis patients (A and B). NL=non-lesional, L=lesional. Relative IL-22 levels (per mg biopsy) in supernatants of ex vivo cultured skin of atopic dermatitis patients in absence (untreated) or presence of CMK389 (C).

FIG. 6. Study design schematic: A randomized, subject and investigator blinded, placebo-controlled multicenter study to assess the efficacy and safety of CMK389 in patients with moderate-to-severe atopic dermatitis.

### DETAILED DESCRIPTION

The present invention demonstrates that IL-18 is a valid target for the treatment and/or prevention of atopic dermatitis or a related condition.

Certain terms used herein are described below. Compounds the present invention are described using standard nomenclature. Unless defined otherwise, all technical and scientific terms used herein have the same meaning as is commonly understood by one of skill in the art to which this invention belongs. The following general definitions shall apply in this specification, unless otherwise specified:

The terms “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound.

The terms “comprising” and “including” are used herein in their open-ended and non-limiting sense unless otherwise noted. As used herein, the term “comprising” encompasses “including” as well as “consisting,” e.g. a composition “comprising” X may consist exclusively of X or may include something additional, e.g., X+Y.

### Treatment of Atopic Dermatitis (AD) or a Related Condition

The present invention demonstrates that IL-18 is a valid target for the treatment and/or prevention of AD or a related condition.

In one aspect, provided herein are IL-18 antagonists, e.g., anti-IL-18 antibody or a fragment thereof, for use in the treatment and/or prevention of AD or a related condition.

In another aspect, provided herein are methods of treatment and/or prevention of AD or a related condition in a subject in need thereof, comprising administering an IL-18 antagonist, e.g., anti-IL-18 antibody or a fragment thereof.

In a further aspect, provided herein are uses of an IL-18 antagonist, e.g., anti-IL-18 antibody or a fragment thereof, in treatment and/or prevention of AD or a related condition.

In a further aspect, provided herein are uses of an IL-18 antagonist, e.g., anti-IL-18 antibody or a fragment thereof, for the manufacture of a medicament for treatment and/or prevention of AD or a related condition.

The therapeutic uses and methods provided herein comprise the administration of a therapeutically effective amount of an IL-18 antagonist, e.g., anti-IL-18 antibody or a fragment thereof, to a subject in need of such treatment.

As used herein, the term “subject” or “patient” includes any human or non-human animal. In a preferred embodiment, the subject is human. The term “non-human animal” includes all vertebrates, e.g., mammals and non-mammals, such as non-human primates, sheep, dogs, cats, horses, cows, chickens, amphibians, reptiles, etc.

As used herein, the terms “subject”, “a subject in need thereof” or the like, mean a human or non-human animal that exhibits one or more symptoms or indicia of AD or a related condition, and/or who has been diagnosed with AD or a related condition. Preferably, the subject is a human. Preferably the subject is a human, e.g., human patient, who has been diagnosed with AD. In certain embodiments, the uses and methods may be used to treat patients that show elevated levels of one or more AD-associated biomarkers (described elsewhere herein). For example, the uses and methods provided herein comprise administering an IL-18 antagonist, e.g., an anti-IL-18 antibody or a fragment thereof, to patients with elevated levels of IgE, hsCRP, CCL17/TARC, CCL22/MDC, CCL26/Eotaxin-3, CD40, IL-24, IL-22, or periostin. In some embodiments, the uses and methods provided herein comprise administering an IL-18 antagonist, e.g., an anti-IL-18 antibody or a fragment thereof, to patients with elevated levels of IL-18 or IL18 BP. In some embodiments, the uses and methods herein may be used to treat AD in children who are <1 year old. For example, the present uses and methods may be used to treat infants who are less than 1 month, 2 months, 3 months, 4 months, 5 months, 6 months, 7 months, 8 months, 9 months, 10 months, 11 months or less than 12 months old. In other embodiments, the present uses and methods may be used to treat children and/or adolescents who are <18 years old. For example, the present methods may be used to treat children or adolescents less than 17 years, 16 years, 15 years, 14 years, 13 years, 12 years, 11 years, 10 years, 9 years, 8 years, 7 years, 6 years, 5 years, 4 years, 3 years, or less than 2 years old.

The term “Atopic dermatitis” (AD) or “eczema”, as used herein, means an inflammatory skin disease characterized by intense pruritus (e.g., severe itch) and by scaly and dry eczematous lesions. The term “Atopic dermatitis” or “eczema” includes, but is not limited to, AD (eczema) caused by or associated with epidermal barrier dysfunction, allergy (e.g., skin allergy, allergy to certain foods, pollen, mold, dust mite, animals, etc.), radiation exposure, and/or asthma. The present disclosure encompasses methods to treat patients with mild, moderate-to-severe or severe AD. As used herein, “moderate-to-severe AD”, is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 10% or more that 20% of the body's skin affected, or 10% of skin area in addition to involvement of the eyes, hands and body folds. Patients affected by moderate-to-severe AD also, in general, have (i) an Investigator's Global Assessment (IGA) score of 3 or 4, (ii) an Eczema Area and Severity Index (EASI) score of at least 10, preferably at least 12, and (iii) itch. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor or any other commonly used therapeutic agent known in the art.

In certain embodiment provided herein are methods to treat moderate-to-severe AD. Suitably, moderate-to-severe atopic dermatitis can be characterized by (i) an Investigator's Global Assessment (IGA) score of 3 or 4, (ii) an Eczema Area and Severity Index (EASI) score of at least 10, preferably at least 12. More suitably, moderate-to-severe atopic dermatitis can be characterized by (i) an Investigator's Global Assessment (IGA) score of 3 or 4, (ii) an Eczema Area and Severity Index (EASI) score of at least 10, more preferably at least 12, and (iii) as a minimum of 10% body-surface area (BSA) affected.

In certain embodiment provided herein are methods to treat both the extrinsic and the intrinsic forms of AD. The extrinsic form of AD associated with IgE-mediated sensitization and increased levels of Th2 cytokines involves 70% to 80% of patients with AD. The intrinsic form without IgE-mediated sensitization involves 20% to 30% of patients with AD; these patients have lower levels of IL-4 and IL-13 than extrinsic AD.

Individuals with atopic dermatitis have an increased risk of developing other conditions related to inflammation. As such, individuals with atopic dermatitis have an increased risk of developing a related condition. The term “related condition”, as used herein, refers to conditions related to inflammation, such as allergy, e.g., skin allergy, food allergy, contact allergy, allergic rhinitis, allergic conjunctivitis, asthma (such as allergic or non-allergic asthma), inflammatory bowel disease, rheumatoid arthritis, and hair loss caused by a malfunctioning immune reaction (alopecia areata), prurigo nodularis, nummular eczema, dyshidrotic eczema, chronic hand eczema, stasis dermatitis, allergic or irritant contact dermatitis, chronic pruritus (such as of hepatic or renal or other origin), nasal polyposis or rhinosinusitis (with or without aspirin intolerance), chronic spontaneous or idiopathic urticaria or other urticaria subtypes, such as urticaria vasculitis, cholinergic urticarial, inducible urticarial, eosinophilic esophagitis, eosinophilic gastritis, eosinophilic colitis, bullous pemphigoid, ichthyoses associated with eczema (such as Netherton syndrome), psoriasis, cutaneous lupus erythematosus, systemic lupus erythematosus, wound healing. The term “related condition”, as used herein, also refers to infectious disorders, such as a skin infection, e.g., cutaneous infection, e.g. eczema herpeticum, e.g., erysipelas, e.g., cellulitis; extra-cutaneous infection, e.g., encephalitis, e.g., endocarditis, e.g., infectious arthropathy, e.g., enterocolitis, e.g., septicemia; respiratory infection, e.g., upper respiratory tract infection, e.g., lower respiratory tract infection, e.g., lung infection; cardiac infection; brain infection; bone infection; and gastrointestinal infection; skin-barrier dysfunction; decreased expression of anti-microbial peptides; aberrant Toll-like receptor signaling and innate immunity; increased colonization with Staphylococcus aureus in lesional and non-lesional skin. The term “related condition”, as used herein, also refers to autoimmune disorders, respiratory disorders, neuropsychiatric disorders, musculoskeletal disorders, and cardiovascular disorders associated with atopic dermatitis.

A skin infection, in particular a bacterial skin infection, is common in AD and is in part due to breaks in the skin from very dry, split skin and from scratching the itchy areas. In certain embodiment provided herein are methods to treat AD, which is accompanied by an infection, in particular a skin infection, more particularly a skin superinfection. In certain embodiment provided herein are methods to treat AD related conditions, wherein the related condition is an infection, in particular a skin infection, more particularly a skin superinfection. In certain embodiments, the infection is (i) a bacterial infection, e.g., staphylococcal bacteria, such as Staphylococcus (S.) aureus, or streptococcal bacteria, such as Streptococcus (S.) epidermitis, and/or (ii) a viral infection, e.g., herpes viral infection. The term “superinfection” as used herein refers to a secondary infection superimposed on an already affected lesional tissue. Superinfection complicates skin lesions and/or leads to colonization or infection by bacteria including commensals. Superinfection could be a viral or a bacterial infection, which is in part due to breaks in the skin from very dry, split skin and from scratching the itchy areas.

The term “treat”, “treating” or “treatment” includes therapeutic treatments, prophylactic treatments and applications in which one reduces the risk that a subject will develop a disorder or other risk factor. Treatment does not require the complete curing of a disorder and encompasses the reduction of the symptoms or underlying risk factors. The invention relates to uses or methods of treatment of AD, e.g., moderate-to-severe AD, wherein the treatment comprises treating or alleviating one or more symptoms of AD. Thus, as used herein, the terms “treat”, “treatment” and “treating” refer to the reduction or amelioration of the progression, severity and/or duration of AD, or the amelioration of one or more symptoms, suitably of one or more discernible symptoms of AD, resulting from the administration of an IL-18 antagonist, e.g., the anti-IL-18 antibody or fragment thereof. In specific embodiments, the terms “treat”, “treatment” and “treating” refer to the amelioration of at least one measurable physical parameter of AD, wherein the physical parameter is not necessarily discernible by the patient.

“Prevention” as used in this context refers to methods which aim to prevent the onset of a disease or its symptoms or which delay the onset of a disease or its symptoms.

As used herein, the terms “effective amount” or “therapeutically effective amount” refer to an amount of a therapy (e.g., a an IL-18 antagonist, e.g., anti-IL-18 antibody or a fragment thereof, e.g., CMK389, or a pharmaceutical composition provided herein) which is sufficient to reduce and/or ameliorate the severity and/or duration of a given condition, disorder, or disease and/or a symptom related thereto. These terms also encompass an amount necessary for the reduction, slowing, or amelioration of the advancement or progression of a given condition, disorder, or disease, reduction, slowing, or amelioration of the recurrence, development or onset of a given condition, disorder or disease, and/or to improve or enhance the prophylactic or therapeutic effect(s) of another therapy (e.g., a therapy other than an IL-18 antagonist, e.g., anti-IL-18 antibody or a fragment thereof). In some aspects, “effective amount” as used herein also refers to the amount of an antagonist, e.g., antibody, described herein to achieve a specified result, for example, improvements in AD-associated parameters, e.g., a decrease in Investigator's Global Assessment (IGA) score; a decrease from baseline in Dermatology Life Quality Index (DLQI); a decrease from baseline in a patient global impression of severity (PGIS); improvement (e.g. decrease from baseline) in a patient global impression of change (PGIC); a decrease in Body Surface Area Involvement of Atopic Dermatitis (BSA) score; a decrease in Eczema Area and Severity Index (EASI) score; a decrease in SCORAD score; and/or a decrease in Pruritus Numeric Rating Scale (NRS) score. In some aspects, “effective amount” as used herein also refers to the amount of an antagonist, e.g., antibody, described herein to achieve a specified result, for example, decrease of the expression level of one or more AD-associated biomarker, in particular one or more AD-associated biomarker selected from the list consisting of CCL17/TARC, IgE (e.g., serum IgE), CCL26/eotaxin-3, CCL22/MDC, hsCRP, CD40, IL-24, IL-22, IL-18 (e.g., serum IL-18, serum free IL-18 (bioactive)), and IL-18BP (e.g., serum IL-18BP), as compared to the level before treatment with the IL-18 antagonist, e.g., the anti-IL-18 antibody or a fragment thereof.

According to certain exemplary embodiments, the present disclosure provides methods for improving one or more AD-associated parameter(s). AD associated parameters and improvements therein are discussed below.

Improvements in AD-associated parameters include, e.g., a decrease in Investigator's Global Assessment (IGA) score; a decrease from baseline in Dermatology Life Quality Index (DLQI); a decrease from baseline in a patient global impression of severity (PGIS); improvement (e.g. decrease from baseline) in a patient global impression of change (PGIC); a decrease in Body Surface Area Involvement of Atopic Dermatitis (BSA) score; a decrease in Eczema Area and Severity Index (EASI) score; a decrease in SCORAD score; and/or a decrease in Pruritus Numeric Rating Scale (NRS) score.

According to certain exemplary embodiments, the uses and methods of the present disclosure results in an improvement in an atopic dermatitis-associated parameter and wherein the improvement in the atopic dermatitis-associated parameter is selected from the group consisting of: (a) a decrease from baseline in Investigator's Global Assessment (IGA) score; (b) a decrease from baseline in Dermatology Life Quality Index (DLQI); (c) improvement (e.g. decrease from baseline) in a patient global impression of severity (PGIS); (d) improvement (e.g. decrease from baseline) in a patient global impression of change (PGIC); (e) a decrease from baseline in Eczema Area and Severity Index (EASI) score; (f) a decrease from baseline in Pruritus Numeric Rating Scale (NRS) score.

In exemplary embodiments, the improvement in an AD-associated parameter is selected from the group consisting of: (a) a decrease from baseline in Investigator's Global Assessment (IGA) score by at least by 2 points, in particular a decrease from baseline in IGA score by at least by 2 points and clear or almost clear status; (b) a decrease from baseline in Dermatology Life Quality Index (DLQI) of at least 30%, preferably at least 40%, more preferably at least 50%; (c) a decrease from baseline in a patient global impression of severity (PGIS) by at least 1 point, preferably by at least 2 points, more preferably by at least 3 points; (d) improvement (e.g. decrease from baseline) in a patient global impression of change (PGIC) by at least 1 point, preferably by at least 2 points, more preferably by at least 3 points; (e) a decrease from baseline in Eczema Area and Severity Index (EASI) score of at least 45%, preferably at least 50%, more preferably at least 60%; (f) percent responders with ≥50% improvement in EASI (EASI50); (g) percent responders with ≥75% improvement in EASI (EASI75); (h) percent responders with ≥90% improvement in EASI (EASI90); (i) percent responders with ≥100% improvement in EASI (EASI100); (f) a decrease from baseline in Pruritus Numeric Rating Scale (NRS) score by at least 3 points, preferably by at least 4 points.

In some embodiments, the improvement in an AD-associated parameter is observed after 4 weeks, after 8 weeks, after 12 weeks, after 16 weeks or more of administration of an IL-18 antagonist, e.g., an anti-IL-18 antibody or a fragment thereof.

### IL-18 Antagonist

The term “IL-18” is synonym to IL-18 polypeptide, Interleukin-18 polypeptide, IFN-gamma-inducing factor or Interferon-gamma-inducing-factor or IFN-γ inducing factor. The term “IL-18” refers to human IL-18 comprising amino acids 37 to 193 of SEQ ID NO: 1. Throughout this specification, the term IL-18 encompasses both pro-IL-18 (precursor of mature IL-18 prior protease cleavage) and mature IL-18 (post protease cleavage) interchangeably unless it is specified that the pro- or mature form is meant. The term “cyno IL-18” refers to cynomolgus monkey IL-18 comprising amino acids 37 to 193 of SEQ ID NO:2.

IL-18 binds with high affinity and signals through the IL-18 receptor (IL-18R), a heteromeric complex of alpha and beta chains encoded by the genes IL18R1 and IL18RAP, respectively (Torigoe K et al (1997) J Biol Chem; 272(41):25737-42). The bioactivity of IL-18 is negatively regulated by the IL-18 binding protein (IL-18BP), a naturally occurring and highly specific inhibitor. This soluble protein forms a complex with free IL-18 preventing its interaction with the IL-18 receptor, thus neutralizing and inhibiting its biological activity (Dinarello CA (2000) Ann Rheum Dis; 59 Suppl 1:i17-20). IL-18BP is a constitutively secreted protein with high affinity binding to IL-18. Alternate mRNA splicing variants of IL-18BP result in four isoforms. The prominent ‘a’ isoform is present in the serum of healthy humans at 20-fold molar excess compared with IL-18 (Dinarello and Kaplanski (2005) Expert Rev Clin Immunol, 1(4), 619-632).

As used herein, the terms “IL-18 antagonist” and “antagonist of IL-18” refer to a molecule that can diminish or inhibit IL-18 activity in vivo. In particular, “IL-18 antagonist” refers to a molecule that inhibits IL-18 dependent signaling activity in the presence of IL-18 in a human cell assay such as IL-18 dependent Interferon-gamma (IFN-γ) production assay in human blood cells. An IL-18 antagonist can bind to an IL-18R or block IL-18 from binding to IL-18R. Suitably, the IL-18 antagonist has the ability to neutralize IL-18, in particular has the ability to neutralize the interaction of the IL-18 polypeptide with the IL-18 receptor. The term “neutralizes” and grammatical variations thereof means throughout this specification, that the biological activity of the target is reduced either totally or partially in the presence of the binding molecule or antibody, as the case may be.

An IL-18 antagonist can be, for example, a small molecule, an anti-IL-18 antibody or an anti-IL-18 antibody fragment (such as an anti-IL-18 antibody or antibody fragment described in WO 2014/037899, GSK-1070806 (GlaxoSmithKline), MEDI-2338 (AstraZeneca; also referred to as AEVI-007), IL-18 binding protein (such as IL-18BP, e.g., tadekinig alfa (r-hIL-18BP from AB2 Bio), IL-18BP fusion protein, such as IL-18BP Fc-fusion or a soluble decoy receptor), an aptamer, an antisense nucleic acid molecule, an interfering RNA, receptor proteins, and the like that can bind specifically to IL-18 or IL-18R.

In one embodiment, the IL-18 antagonist binds to an IL-18R. In one embodiment, the IL-18 antagonist binds to IL-18. In a preferred embodiment, the IL-18 antagonist specifically binds IL-18, and does not bind the IL-18/IL-18 binding protein (IL-18 BP) complex.

In one embodiment, the IL-18 antagonist is IL-18BP or IL-18BP fusion protein. The term “IL-18BP” or “IL-18 binding protein” refers to human, murine or viral IL-18 binding proteins in every isoform, whether naturally occurring, isolated or engineered such as the IL-18BP disclosed in WO2001/085201 which describes analogues of IL-18BP (“muteins”) wherein one or more amino acids are inserted, replaced by different conservative substitutions or deleted, IL-18BP fused protein (e.g. fused protein of an IL-18BP and an immunoglobulin heavy chain region or Fc) and functional derivatives such as PEG-ylated IL-18BP.

In a preferred embodiment, the IL-18 antagonist is an anti-IL-18 antibody or an anti-IL-18 antibody fragment. Suitably, the anti-IL-18 antibody of the disclosure or an antibody fragment is a therapeutic antibody.

The term “antibody” refers to an intact immunoglobulin or a functional fragment thereof. Naturally occurring antibodies typically comprise a tetramer which is usually composed of at least two heavy (H) chains and at least two light (L) chains. Each heavy chain is comprised of a heavy chain variable region (abbreviated herein as VH) and a heavy chain constant region, usually comprised of three domains (CH1, CH2 ad CH3). Heavy chains can be of any isotype, including IgG (IgG1, IgG2, IgG3 and IgG4 subtypes), IgA (IgA1 and IgA2 subtypes), IgM and IgE. Each light chain is comprised of a light chain variable region (abbreviated herein as VL) and a light chain constant region (CL). Light chain includes kappa chains and lambda chains. The heavy and light chain variable region is typically responsible for antigen recognition, whilst the heavy and light chain constant region may mediate the binding of the immunoglobulin to host tissues or factors, including various cells of the immune system (e.g., effector cells) and the first component (Clq) of the classical complement system. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

The term “antigen-binding portion” of an antibody (or simply “antigen portion”), as used herein, refers to full length or one or more fragments of an antibody that retain the ability to specifically bind to IL-18. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature; 341:544-546), which consists of a VH domain; and an isolated complementarity determining region (CDR). As used herein, the term” antibody fragment” refers to one or more fragments of an antibody that retain the ability to specifically bind to IL-18 (“antigen-binding portion”).

Furthermore, although the two domains of the Fv fragment, VL and VH, are coded by separate genes, they can be joined, using recombinant methods, by a flexible linker that enables them to be made as a single protein chain in which the VL and VH regions pair to form monovalent molecules (known as single chain Fv (scFv); see e.g., Bird et al., (1988) Science 242:423-426; and Huston et al., (1988) Proc Natl Acad Sci. 85:5879-5883). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

The term “anti-IL-18 antibody or anti-IL18 antibody fragment” or “anti-IL-18 antibody or a fragment thereof” as used herein refers to an antibody, or fragment thereof, which comprises an IL-18 binding domain.

Suitably, the IL-18 antagonist of the disclosure is an isolated antibody or a fragment thereof. The term “isolated” means throughout this specification, that the immunoglobulin, antibody or polynucleotide, as the case may be, exists in a physical milieu distinct from that in which it may occur in nature. An isolated antibody that specifically binds the IL-18 may, however, have cross-reactivity to other antigens, such as IL-18 from other species (e.g. cynomolgus monkey (cyno) IL-18). Moreover, an isolated antibody may be substantially free of other cellular material and/or chemicals.

Suitably, the IL-18 antagonist of the disclosure is a monoclonal antibody or a fragment thereof. The terms “monoclonal antibody” as used herein refer to a preparation of antibody molecules of single molecular composition. A monoclonal antibody composition displays a single binding specificity and affinity for a particular epitope.

Suitably, the IL-18 antagonist of the disclosure is a fully human, humanized or chimeric antibody or a fragment thereof.

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik, et al., (2000) J Mol Biol; 296:57-86). The human antibodies of the disclosure may include amino acid residues not encoded by human sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human sequences.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

In a preferred embodiment, the IL-18 antagonist is an anti-IL-18 antibody or an antibody fragment described in WO 2014/037899, the entire contents of which are hereby incorporated by reference. In another embodiment, the IL-18 antagonist is an anti-IL-18 antibody GSK-1070806 (GlaxoSmithKline) or a fragment thereof. In another embodiment, the IL-18 antagonist is an anti-IL-18 antibody MEDI-2338 (AstraZeneca; also referred to as AEVI-007) or a fragment thereof.

In one embodiment, the present invention relates to an IL-18 antagonist, e.g., an anti-IL-18 antibody or a fragment thereof, wherein the I1-18 antagonist specifically binds IL-18. In a more specific embodiment, the present invention relates to an I1-18 antagonist, e.g., an anti-IL-18 antibody or a fragment thereof, wherein the IL-18 antagonist specifically binds IL-18, but does not bind the IL-18/IL-18 binding protein complex.

As used herein, “an IL-18 antagonist that specifically binds IL-18” is intended to refer to a compound or a molecule that binds to human IL-18 with a KD of a 100 nM or less, 10 nM or less, 1 nM or less, 100 pM or less, 10 pM or less, in particular as measured by SET. As used herein, “an antibody or a fragment thereof that specifically binds IL-18” is intended to refer to an antibody or a fragment thereof that binds to human IL-18 with a KD of a 100 nM or less, 10 nM or less, 1 nM or less, 100 pM or less, 10 pM or less, in particular as measured by SET.

In one embodiment, the IL-18 antagonist, e.g., an anti-IL-18 antibody or a fragment thereof, binds human IL-18 with a dissociation constant (KD) of 100 pM or less, e.g., 50 pM or less, 25 pM or less, 10 pM or less, 5 pM or less, preferably with a KD of 0.5 pM to 20 pM, in particular as measured by SET.

An antibody or a fragment thereof that “cross-reacts with an antigen other than IL-18” is intended to refer to an antibody or a fragment thereof that binds that antigen with a KD of a 100 nM or less, 10 nM or less, 1 nM or less. An antibody or a fragment thereof that “does not cross-react with a particular antigen” is intended to refer to a binding molecule that exhibits essentially undetectable binding against these proteins in standard binding assays.

As used herein, the term “does not bind the IL-18/IL-18 binding protein (IL-18 BP) complex” is intended to refer to an antibody or a fragment thereof that binds to the IL-18/IL-18 binding protein (IL-18 BP) complex with a KD of 1×10⁻⁵ M or greater.

As used herein, the term “affinity” refers to the strength of interaction between an antibody or a fragment thereof and an antigen at single antigenic sites. Within each antigenic site, the variable region of the antibody “arm” interacts through weak non-covalent forces with antigen at numerous sites; the more interactions, the stronger the affinity. As used herein, the term “high affinity” for an antibody refers to an antibody having a KD of 1 nM or less for a target antigen.

The term “kassoc” or “ka” or “kon”, as used herein, is intended to refer to the association rate of a particular antibody-antigen interaction, whereas the term “kdis” or “kd” or “koff” as used herein, is intended to refer to the dissociation rate of a particular antibody-antigen interaction. The term “KD”, as used herein, is intended to refer to the dissociation constant, which is obtained from the ratio of kd to ka (i.e. kd/ka) and is expressed as a molar concentration (M). KD values for antibodies can be determined using methods well established in the art. Methods for determining the KD of an antibody include measuring surface plasmon resonance using a biosensor system such as a Biacore™ system, or measuring affinity in solution by solution equilibrium titration (SET).

In one embodiment, the IL-18 antagonist, e.g., an anti-IL-18 antibody or a fragment thereof, inhibits IL-18-induced interferon gamma (IFN-γ) production from KG-1 cells with IC50 of less than 5 nM, e.g., 0.1 to 1 nM.

In a further embodiment, the IL-18 antagonist, e.g., an anti-IL-18 antibody or a fragment thereof, inhibits IL-18-induced interferon gamma (IFN-γ) production in whole blood with IC50 of less than 150 nM, e.g., 5 to 10 nM.

The term “epitope” is the part of an antigen that is recognized by the immune system, such as an antibody or a fragment thereof. Within the present specification, the term “epitope” is used interchangeably for both conformational epitopes and linear epitopes. A conformational epitope is composed of discontinuous sections of the antigen's amino acid sequence, whilst a linear epitope is formed by a continuous sequence of amino acids from the antigen.

In one embodiment, the IL-18 antagonist of the disclosure, in particular the IL-18 antagonist which binds IL-18 and does not bind the IL-18/IL-18 binding protein (IL-18 BP) complex, binds with an IL-18 epitope on IL-18 as defined with reference to SEQ ID NO:1, wherein the epitope:
a. is comprised within the following amino acids of IL-18 as defined with reference to SEQ ID NO:1:
   i. amino acids 41 and 42 and amino acids 87 to 97; or
   ii. amino acids 138 to 160; or
   iii. amino acids 177 to 181; or
   iv. amino acids 41 and 42, amino acids 87 to 97, amino acids 138 to 160 and amino acids 177 to 181; or
   v. amino acids 41, 42, 87; 89; 90; or
   vi. amino acids 93, 94; 95, 96; or
   vii. amino acids 140; 141; 150; 177; or
   viii. amino acids 92; 93; 94; 138; 140; 152; 157; or
   ix. amino acids 142; 143; 150; 152; or
   x. amino acids 143; 144; 145; 177; 180; or
   xi. amino acids 41, 42, 87; 89; 90; 93, 94; 95, 96; 140; 141; 150; 177; or
   xii. amino acids 92; 93; 94; 138; 140; 142; 143; 144; 145; 150; 152; 157; 177; 180; or
   xiii. amino acids 41; 42, 87; 89; 90; 92; 93, 94; 95, 96; 138; 140; 141; 142; 143; 144; 145; 150; 152; 157; 177; 180; or
b. comprises at least one, two, three or four of the amino acids as defined in any one of the groups (i) to (xiii) listed in a); or
c. comprises the amino acids as defined in any one of the groups (iv) to (xii) listed in a).

In another embodiment, the IL-18 antagonist of the disclosure, in particular the IL-18 antagonist which binds IL-18 and does not bind the IL-18/IL-18 binding protein (IL-18 BP) complex, binds with an IL-18 epitope on IL-18 as defined with reference to SEQ ID NO:1, wherein the epitope comprises amino acids Arg140 and Glu152. In one embodiment the epitope further comprises any one or more of amino acids Gln92, Pro93, Gly95, Pro143, Glu157 or Glu177. In another embodiment the epitope further comprises any one or more of amino acids Lys89, Arg94, Met96, Phe138, Ser141, Gly144, His145, Asp146, Gln150 or Leu180.

In one embodiment, the IL-18 antagonist of the disclosure, in particular the IL-18 antagonist which binds IL-18 and does not bind the IL-18/IL-18 binding protein (IL-18 BP) complex, does not bind the IL-18/IL-18 binding protein isoform a or isoform c (IL-18 BPa or IL-18BPc) complex.

In another embodiment, In one embodiment, the IL-18 antagonist of the disclosure, in particular the IL-18 antagonist which binds IL-18 and does not bind the IL-18/IL-18 binding protein (IL-18 BP) complex, and does not bind the IL-18/IL-18 binding protein isoform a or isoform c (IL-18 BPa or IL-18BPc) complex, wherein the IL-18 antagonist binds with an IL-18 epitope on IL-18 as defined with reference to SEQ ID NO:1, wherein the epitope comprises amino acids Arg140 and Glu152. In one embodiment the epitope further comprises any one or more of amino acids Gln92, Pro93, Gly95, Pro143, Glu157 or Glu177.

Suitably, the IL-18 antagonists of the present disclosure include antibodies or fragments thereof, as described hereinafter.

The precise amino acid sequence boundaries of a given complementarity determining region (CDR) can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

Throughout this specification, complementarity determining regions (“CDR”) are defined according to the Kabat definition unless specified that the CDR are defined according to the Chothia definition or by both definitions together. By convention, the CDR regions in the heavy chain are typically referred to as H-CDR1, H-CDR2 and H-CDR3 and in the light chain as L-CDR1, LCDR2 and L-CDR3. They are numbered sequentially in the direction from the amino terminus to the carboxy terminus.

A “conservative variant” of a sequence encoding a binding molecule, an antibody or a fragment thereof refers to a sequence comprising conservative amino acid modifications. “Conservative amino acid modifications” are intended to refer to amino acid modifications that do not significantly affect or alter the binding characteristics of the antibody containing the amino acid sequence. Such conservative modifications include amino acid substitutions, additions and deletions. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Modifications can be introduced into a binding protein of the invention by standard techniques known in the art, such as site-directed mutagenesis and PCR-mediated mutagenesis. Conservative amino acid substitution can also encompass non-naturally occurring amino acid residues which are typically incorporated by chemical peptide synthesis rather than by synthesis in biological systems. Non-naturally occurring amino acids include, but are not limited to, peptidomimetic, reversed or inverted forms of amino acid moieties.

The term “identity” refers to the similarity between at least two different sequences. This identity can be expressed as a percent identity and determined by standard alignment algorithms, for example, the Basic Local Alignment Tool (BLAST) (Altshul et al., (1990) J Mol Biol; 215:403-410); the algorithm of Needleman et al., (1970) J Mol Biol; 48:444-453 or the algorithm of Meyers et al., (1988) Comput Appl Biosci; 4:11-17). As used herein, the percent identity between the two sequences is a function of the number of identical positions shared by the sequences (i. e., % identity=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described below. The percent identity between two amino acid sequences can be determined using the algorithm of E. Meyers and W. Miller, (1988) Comput. Appl. Biosci 4:11-17 which has been incorporated into the ALIGN program (version 2.0), using a PAM120 weight residue table, a gap length penalty of 12 and a gap penalty of 4. Alternatively, the percent identity between two amino acid sequences can be determined using the Needleman and Wunsch (1970) J Mol Biol 48:444-453, algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof,
(ii) a heavy chain variable region H-CDR2 comprising SEQ ID NO: 4 or SEQ ID NO: 9 or SEQ ID NO: 10 or SEQ ID NO: 11 or SEQ ID NO: 12 or SEQ ID NO: 13 or a conservative variant thereof,
(iii) a heavy chain variable region H-CDR3 comprising SEQ ID NO: 5 or a conservative variant thereof,
(iv) a light chain variable region L-CDR1 comprising SEQ ID NO: 6 or a conservative variant thereof,
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof,
(ii) a heavy chain variable region H-CDR2 comprising SEQ ID NO: 4 or a conservative variant thereof,
(iii) a heavy chain variable region H-CDR3 comprising SEQ ID NO: 5 or a conservative variant thereof,
(iv) a light chain variable region L-CDR1 comprising SEQ ID NO: 6 or a conservative variant thereof,
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof, and
(ii) a heavy chain variable region H-CDR2 comprising SEQ ID NO: 9 or a conservative variant thereof, and
(iii) a heavy chain variable region H-CDR3 comprising SEQ ID NO: 5 or a conservative variant thereof, and
(iv) a light chain variable region L-CDR1 comprising SEQ ID NO: 6 or a conservative variant thereof, and
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, and that the IL-18 antagonist binds to an epitope comprising Arg140 and Glu152 on IL-18 as defined with reference to SEQ ID NO:1, wherein the IL-18 antagonist comprises:
i. a heavy chain variable region H-CDR1 comprising SEQ ID NO: 3 or a conservative variant thereof, and
ii. a heavy chain variable region H-CDR2 comprising SEQ ID NO: 9 or a conservative variant thereof, and
iii. a heavy chain variable region H-CDR3 comprising SEQ ID NO: 5 or a conservative variant thereof, and
iv. a light chain variable region L-CDR1 comprising SEQ ID NO: 6 or a conservative variant thereof, and
v. a light chain variable region L-CDR2 comprising SEQ ID NO: 7 or a conservative variant thereof, and
vi. a light chain variable region L-CDR3 comprising SEQ ID NO: 8 or a conservative variant thereof.

Preferably this IL-18 antagonist is an isolated human antibody, more preferably an isolated fully human antibody or fragment thereof, more preferably an isolated fully human monoclonal antibody or fragment thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof, and
(ii) a heavy chain variable region H-CDR2 comprising SEQ ID NO: 10 or a conservative variant thereof, and
(iii) a heavy chain variable region H-CDR3 comprising SEQ ID NO: 5 or a conservative variant thereof, and
(iv) a light chain variable region L-CDR1 comprising SEQ ID NO: 6 or a conservative variant thereof, and
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof, and
(ii) a heavy chain variable region H-CDR2 comprising SEQ ID NO: 11 or a conservative variant thereof, and
(iii) a heavy chain variable region H-CDR3 comprising SEQ ID NO: 5 or a conservative variant thereof, and
(iv) a light chain variable region L-CDR1 comprising SEQ ID NO: 6 or a conservative variant thereof, and
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof, and
(ii) a heavy chain variable region H-CDR2 comprising SEQ ID NO: 12 or a conservative variant thereof, and
(iii) a heavy chain variable region H-CDR3 comprising SEQ ID NO: 5 or a conservative variant thereof, and
(iv) a light chain variable region L-CDR1 comprising SEQ ID NO: 6 or a conservative variant thereof, and
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 3 or a conservative variant thereof, and
(ii) a heavy chain variable region H-CDR2 comprising SEQ ID NO: 13 or a conservative variant thereof, and
(iii) a heavy chain variable region H-CDR3 comprising SEQ ID NO: 5 or a conservative variant thereof, and
(iv) a light chain variable region L-CDR1 comprising SEQ ID NO: 6 or a conservative variant thereof, and
(v) a light chain variable region L-CDR2 comprising SEQ ID NO: 7 or a conservative variant thereof, and
(vi) a light chain variable region L-CDR3 comprising SEQ ID NO: 8 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, and that IL-18 antagonist binds to an epitope comprising Arg140 and Glu152 on IL-18 as defined with reference to SEQ ID NO:1 wherein the IL-18 antagonist comprises:
i. a heavy chain variable region H-CDR1 comprising SEQ ID NO: 3 or a conservative variant thereof, and
ii. a heavy chain variable region H-CDR2 comprising SEQ ID NO: 13 or a conservative variant thereof, and
iii. a heavy chain variable region H-CDR3 comprising SEQ ID NO: 5 or a conservative variant thereof, and
iv. a light chain variable region L-CDR1 comprising SEQ ID NO: 6 or a conservative variant thereof, and
v. a light chain variable region L-CDR2 comprising SEQ ID NO: 7 or a conservative variant thereof, and
vi. a light chain variable region L-CDR3 comprising SEQ ID NO: 8 or a conservative variant thereof.

Preferably this IL-18 antagonist is an isolated human antibody, more preferably an isolated fully human antibody or fragment thereof, more preferably an isolated fully human monoclonal antibody or fragment thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 74 or a conservative variant thereof, and
(ii) a heavy chain variable region H-CDR2 comprising SEQ ID NO: 75 or SEQ ID NO: 76 or SEQ ID NO: 77 or SEQ ID NO: 78 or a conservative variant thereof, and
(iii) a heavy chain variable region H-CDR3 comprising SEQ ID NO: 79 or a conservative variant thereof, and
(iv) a light chain variable region L-CDR1 comprising SEQ ID NO: 80 or a conservative variant thereof, and
(v) a light chain variable region L-CDR2 comprising SEQ ID NO: 81 or a conservative variant thereof, and
(vi) a light chain variable region L-CDR3 comprising SEQ ID NO: 82 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 74 or a conservative variant thereof, and
(ii) a heavy chain variable region H-CDR2 comprising SEQ ID NO: 75 or a conservative variant thereof, and
(iii) a heavy chain variable region H-CDR3 comprising SEQ ID NO: 79 or a conservative variant thereof, and
(iv) a light chain variable region L-CDR1 comprising SEQ ID NO: 80 or a conservative variant thereof, and
(v) a light chain variable region L-CDR2 comprising SEQ ID NO: 81 or a conservative variant thereof, and
(vi) a light chain variable region L-CDR3 comprising SEQ ID NO: 82 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 74 or a conservative variant thereof, and
(ii) a heavy chain variable region H-CDR2 comprising SEQ ID NO: 76 or a conservative variant thereof, and
(iii) a heavy chain variable region H-CDR3 comprising SEQ ID NO: 79 or a conservative variant thereof, and
(iv) a light chain variable region L-CDR1 comprising SEQ ID NO: 80 or a conservative variant thereof, and
(v) a light chain variable region L-CDR2 comprising SEQ ID NO: 81 or a conservative variant thereof, and
(vi) a light chain variable region L-CDR3 comprising SEQ ID NO: 82 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 74 or a conservative variant thereof, and
(ii) a heavy chain variable region H-CDR2 comprising SEQ ID NO: 77 or a conservative variant thereof, and
(iii) a heavy chain variable region H-CDR3 comprising SEQ ID NO: 79 or a conservative variant thereof, and
(iv) a light chain variable region L-CDR1 comprising SEQ ID NO: 80 or a conservative variant thereof, and
(v) a light chain variable region L-CDR2 comprising SEQ ID NO: 81 or a conservative variant thereof, and
(vi) a light chain variable region L-CDR3 comprising SEQ ID NO: 82 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 74 or a conservative variant thereof, and
(ii) a heavy chain variable region H-CDR2 comprising SEQ ID NO: 78 or a conservative variant thereof, and
(iii) a heavy chain variable region H-CDR3 comprising SEQ ID NO: 79 or a conservative variant thereof, and
(iv) a light chain variable region L-CDR1 comprising SEQ ID NO: 80 or a conservative variant thereof, and
(v) a light chain variable region L-CDR2 comprising SEQ ID NO: 81 or a conservative variant thereof, and
(vi) a light chain variable region L-CDR3 comprising SEQ ID NO: 82 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 106 or a conservative variant thereof, and
(ii) a heavy chain variable region H-CDR2 comprising SEQ ID NO: 107 or SEQ ID NO: 122 or a conservative variant thereof, and
(iii) a heavy chain variable region H-CDR3 comprising SEQ ID NO: 108 or a conservative variant thereof, and
(iv) a light chain variable region L-CDR1 comprising SEQ ID NO: 109 or a conservative variant thereof, and
(v) a light chain variable region L-CDR2 comprising SEQ ID NO: 110 a conservative variant thereof, and
(vi) a light chain variable region L-CDR3 comprising SEQ ID NO: 111 or SEQ ID NO: 126 a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 106 or a conservative variant thereof, and
(ii) a heavy chain variable region H-CDR2 comprising SEQ ID NO: 107 or a conservative variant thereof, and
(iii) a heavy chain variable region H-CDR3 comprising SEQ ID NO: 108 or a conservative variant thereof, and
(iv) a light chain variable region L-CDR1 comprising SEQ ID NO: 109 or a conservative variant thereof, and
(v) a light chain variable region L-CDR2 comprising SEQ ID NO: 110 or a conservative variant thereof, and
(vi) a light chain variable region L-CDR3 comprising SEQ ID NO: 111 or a conservative variant thereof.

Preferably, this IL-18 antagonist is an isolated human antibody, more preferably an isolated fully human antibody or fragment thereof, more preferably an isolated fully human monoclonal antibody or fragment thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 106 or a conservative variant thereof, and
(ii) a heavy chain variable region H-CDR2 comprising SEQ ID NO: 122 or a conservative variant thereof, and
(iii) a heavy chain variable region H-CDR3 comprising SEQ ID NO: 108 or a conservative variant thereof, and
(iv) a light chain variable region L-CDR1 comprising SEQ ID NO: 109 or a conservative variant thereof, and
(v) a light chain variable region L-CDR2 comprising SEQ ID NO: 110 or a conservative variant thereof, and
(vi) a light chain variable region L-CDR3 comprising SEQ ID NO: 126 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 120 or a conservative variant thereof, and
(ii) a heavy chain variable region H-CDR2 comprising SEQ ID NO: 121 or a conservative variant thereof, and
(iii) a heavy chain variable region H-CDR3 comprising SEQ ID NO: 123 or a conservative variant thereof, and
(iv) a light chain variable region L-CDR1 comprising SEQ ID NO: 124 or a conservative variant thereof, and
(v) a light chain variable region L-CDR2 comprising SEQ ID NO: 125 or a conservative variant thereof, and
(vi) a light chain variable region L-CDR3 comprising SEQ ID NO: 127 or SEQ ID NO: 128 or SEQ ID NO: 129 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 120 or a conservative variant thereof, and
(ii) a heavy chain variable region H-CDR2 comprising SEQ ID NO: 121 or a conservative variant thereof, and
(iii) a heavy chain variable region H-CDR3 comprising SEQ ID NO: 123 or a conservative variant thereof, and
(iv) a light chain variable region L-CDR1 comprising SEQ ID NO: 124 or a conservative variant thereof, and
(v) a light chain variable region L-CDR2 comprising SEQ ID NO: 125 or a conservative variant thereof, and
(vi) a light chain variable region L-CDR3 comprising SEQ ID NO: 127 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 120 or a conservative variant thereof, and
(ii) a heavy chain variable region H-CDR2 comprising SEQ ID NO: 121 or a conservative variant thereof, and
(iii) a heavy chain variable region H-CDR3 comprising SEQ ID NO: 123 or a conservative variant thereof, and
(iv) a light chain variable region L-CDR1 comprising SEQ ID NO: 124 or a conservative variant thereof, and
(v) a light chain variable region L-CDR2 comprising SEQ ID NO: 125 or a conservative variant thereof, and
(vi) a light chain variable region L-CDR3 comprising SEQ ID NO: 128 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 120 or a conservative variant thereof, and
(ii) a heavy chain variable region H-CDR2 comprising SEQ ID NO: 121 or a conservative variant thereof, and
(iii) a heavy chain variable region H-CDR3 comprising SEQ ID NO: 123 or a conservative variant thereof, and
(iv) a light chain variable region L-CDR1 comprising SEQ ID NO: 124 or a conservative variant thereof, and
(v) a light chain variable region L-CDR2 comprising SEQ ID NO: 125 or a conservative variant thereof, and
(vi) a light chain variable region L-CDR3 comprising SEQ ID NO: 129 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 59 or SEQ ID NO: 65 or SEQ ID NO: 66 or a conservative variant thereof, and
(ii) a heavy chain variable region H-CDR2 comprising SEQ ID NO: 60 or SEQ ID NO: 67 or a conservative variant thereof, and
(iii) a heavy chain variable region H-CDR3 comprising SEQ ID NO: 61 or a conservative variant thereof, and
(iv) a light chain variable region L-CDR1 comprising SEQ ID NO: 62 or a conservative variant thereof, and
(v) a light chain variable region L-CDR2 comprising SEQ ID NO: 63 or a conservative variant thereof, and
(vi) a light chain variable region L-CDR3 comprising SEQ ID NO: 64 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 59 or a conservative variant thereof, and
(ii) a heavy chain variable region H-CDR2 comprising SEQ ID NO: 60 or a conservative variant thereof, and
(iii) a heavy chain variable region H-CDR3 comprising SEQ ID NO: 61 or a conservative variant thereof, and
(iv) a light chain variable region L-CDR1 comprising SEQ ID NO: 62 or a conservative variant thereof, and
(v) a light chain variable region L-CDR2 comprising SEQ ID NO: 63 or a conservative variant thereof, and
(vi) a light chain variable region L-CDR3 comprising SEQ ID NO: 64 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 65 or a conservative variant thereof, and
(ii) a heavy chain variable region H-CDR2 comprising SEQ ID NO: 60 or a conservative variant thereof, and
(iii) a heavy chain variable region H-CDR3 comprising SEQ ID NO: 61 or a conservative variant thereof, and
(iv) a light chain variable region L-CDR1 comprising SEQ ID NO: 62 or a conservative variant thereof, and
(v) a light chain variable region L-CDR2 comprising SEQ ID NO: 63 or a conservative variant thereof, and
(vi) a light chain variable region L-CDR3 comprising SEQ ID NO: 64 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 66 or a conservative variant thereof, and
(ii) a heavy chain variable region H-CDR2 comprising SEQ ID NO: 67 or a conservative variant thereof, and
(iii) a heavy chain variable region H-CDR3 comprising SEQ ID NO: 61 or a conservative variant thereof, and
(iv) a light chain variable region L-CDR1 comprising SEQ ID NO: 62 or a conservative variant thereof, and
(v) a light chain variable region L-CDR2 comprising SEQ ID NO: 63 or a conservative variant thereof, and
(vi) a light chain variable region L-CDR3 comprising SEQ ID NO: 64 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 68 or a conservative variant thereof, and
(ii) a heavy chain variable region H-CDR2 comprising SEQ ID NO: 69 or a conservative variant thereof, and
(iii) a heavy chain variable region H-CDR3 comprising SEQ ID NO: 70 or a conservative variant thereof, and
(iv) a light chain variable region L-CDR1 comprising SEQ ID NO: 71 or a conservative variant thereof, and
(v) a light chain variable region L-CDR2 comprising SEQ ID NO: 72 or a conservative variant thereof, and
(vi) a light chain variable region L-CDR3 comprising SEQ ID NO: 73 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(i) a heavy chain variable region H-CDR1 comprising SEQ ID NO: 162 or a conservative variant thereof, and
(ii) a heavy chain variable region H-CDR2 comprising SEQ ID NO: 163 or a conservative variant thereof, and
(iii) a heavy chain variable region H-CDR3 comprising SEQ ID NO: 164 or a conservative variant thereof, and
(iv) a light chain variable region L-CDR1 comprising SEQ ID NO: 165 or a conservative variant thereof, and
(v) a light chain variable region L-CDR2 comprising SEQ ID NO: 166 or a conservative variant thereof, and
(vi) a light chain variable region L-CDR3 comprising SEQ ID NO: 167 or a conservative variant thereof.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(a) a heavy chain variable region (VH) comprising SEQ ID NO: 14 or a conservative variant thereof or a sequence at least 90% identical thereto, and
(b) a light chain variable region (VL) comprising SEQ ID NO: 16 or a conservative variant thereof or a sequence at least 90% identical thereto,
   and wherein the heavy chain variable region (VH) comprises:
i. a H-CDR1 corresponding to amino acids 26 to 35 of SEQ ID NO: 14; and
ii. a H-CDR2 corresponding to amino acids 50 to 66 of SEQ ID NO: 14; and
iii. a H-CDR3 corresponding to amino acids 99 to 108 of SEQ ID NO: 14;
   and wherein the light chain variable region (VL) comprises:
iv. a L-CDR1 corresponding to amino acids 23 to 35 of SEQ ID NO: 16; and
v. a L-CDR2 corresponding to amino acids 51 to 57 of SEQ ID NO: 16; and
vi. a L-CDR3 corresponding to amino acids 90 to 100 of SEQ ID NO: 16.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(a) a heavy chain variable region (VH) comprising SEQ ID NO: 18 or a conservative variant thereof or a sequence at least 90% identical thereto, and
(b) a light chain variable region (VL) comprising SEQ ID NO: 20 or a conservative variant thereof or a sequence at least 90% identical thereto,
   and wherein the heavy chain variable region (VH) comprises:
i. a H-CDR1 corresponding to amino acids 26 to 35 of SEQ ID NO: 18; and
ii. a H-CDR2 corresponding to amino acids 50 to 66 of SEQ ID NO: 18; and
iii. a H-CDR3 corresponding to amino acids 99 to 108 of SEQ ID NO: 18;
   and wherein the light chain variable region (VL) comprises:
iv. a L-CDR1 corresponding to amino acids 23 to 35 of SEQ ID NO: 20; and
v. a L-CDR2 corresponding to amino acids 51 to 57 of SEQ ID NO: 20; and
vi. a L-CDR3 corresponding to amino acids 90 to 100 of SEQ ID NO: 20.

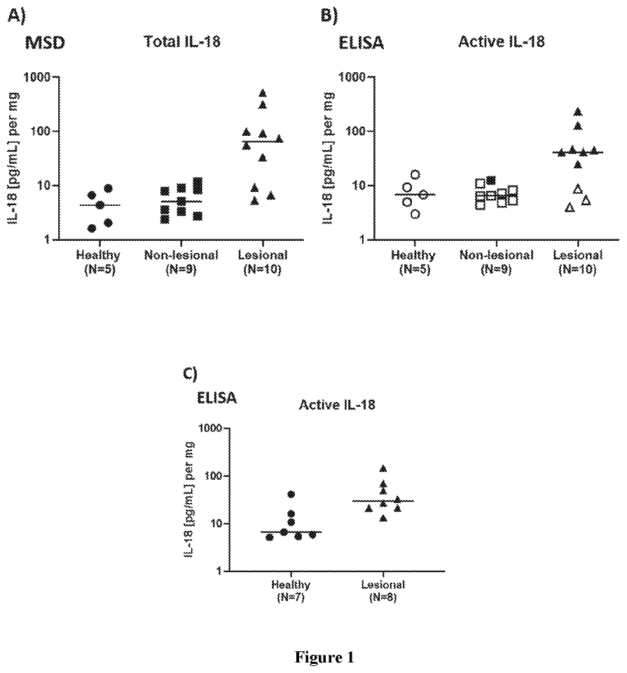

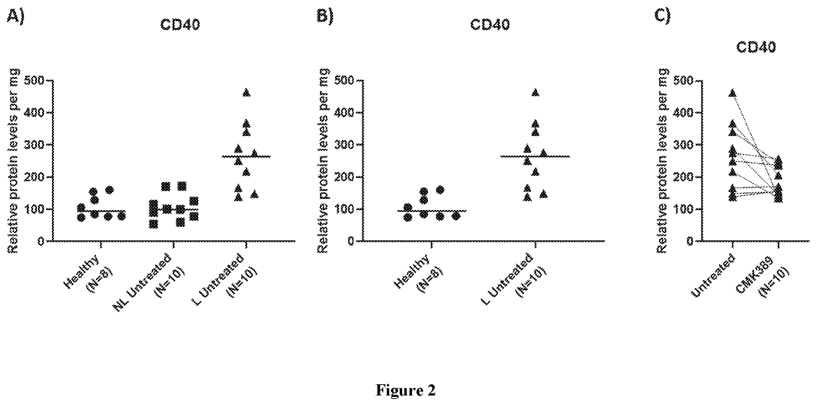

Given that each of these human antibodies can bind to IL18 and that antigen-binding specificity is provided primarily by the CDR1, 2 and 3 regions, the H-CDR1, 2 and 3 sequences and L-CDR1, 2 and 3 sequences can be “mixed and matched” (i.e., CDRs from different human antibodies can be mixed and matched, each antibody containing a H-CDR1, 2 and 3 set and a L-CDR1, 2 and 3 set creates other anti-IL18 binding molecules of the invention). IL18 binding of such “mixed and matched” antibodies can be tested using the binding assays in the Examples (e.g., ELISAs). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for human antibodies of the present invention (FIGS. 1 and 2).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises: (a) a VH amino acid sequence selected from the sequences shown in SEQ ID NOs: 14, 18, 22, 25, 28, 31, 34, 37, 40, 83, 87, 90, 93, 112, 130 and 138, and (b) a VL amino acid sequence selected from the sequences shown in SEQ ID NOs: 16, 20, 85, 114, 132, 140, 147 and 153. Other antibodies of the disclosure include amino acids that have been mutated by amino acid deletion, insertion or substitution, yet have at least 60, 70, 80, 90 or 95 percent identity in the CDR regions with the CDR regions depicted in the sequences described above. In some embodiments, it include mutant amino acid sequences wherein no more than 1, 2, 3, 4 or 5 amino acids have been mutated by amino acid deletion, insertion or substitution in the CDR regions when compared with the CDR regions depicted in the sequences described above.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 28 or a conservative variant thereof or a sequence at least 90% identical thereto, and (b) a light chain variable region (VL) amino acid sequence comprising SEQ ID NO: 16 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 28 or a conservative variant thereof or a sequence at least 90% identical thereto, and (b) a light chain variable region (VL) amino acid sequence comprising SEQ ID NO: 16 or a conservative variant thereof or a sequence at least 90% identical thereto, wherein amino acid asparagine (Asn; N) in position 30 with reference to SEQ ID NO: 28 is replaced by an amino acid selected from lysine (Lys; K), serine (Ser; S), threonine (Thr; T), alanine (Ala; A), glutamate (Glu; E), histidine (His; H), leucine (Leu; L), glutamine (Gln; Q), arginine (Arg; R), valine (Val; V), tyrosine (Tyr; Y), isoleucine (Ile; I) and glycine (Gly; G).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 14 or a conservative variant thereof or a sequence at least 90% identical thereto, and (b) a light chain variable region (VL) amino acid sequence comprising SEQ ID NO: 16 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 14 or a conservative variant thereof or a sequence at least 90% identical thereto, and (b) a light chain variable region (VL) amino acid sequence comprising SEQ ID NO: 16 or a conservative variant thereof or a sequence at least 90% identical thereto, wherein amino acid lysine (Lys; K) in position 30 with reference to SEQ ID NO: 14 is replaced by an amino acid selected from asparagine (Asn; N), serine (Ser; S), threonine (Thr; T), alanine (Ala; A), glutamate (Glu; E), histidine (His; H), leucine (Leu; L), glutamine (Gln; Q), arginine (Arg; R), valine (Val; V), tyrosine (Tyr; Y), isoleucine (Ile; I) and glycine (Gly; G).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 18 or a conservative variant thereof or a sequence at least 90% identical thereto, and (b) a light chain variable region (VL) amino acid sequence comprising SEQ ID NO: 20 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 40 or a conservative variant thereof or a sequence at least 90% identical thereto, and (b) a light chain variable region (VL) amino acid sequence comprising SEQ ID NO: 20 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 40 or a conservative variant thereof or a sequence at least 90% identical thereto, and (b) a light chain variable region (VL) amino acid sequence comprising SEQ ID NO: 20 or a conservative variant thereof or a sequence at least 90% identical thereto, wherein
i. amino acid glutamate (Glu; E) in position 1 with reference to SEQ ID NO: 40 is replaced by amino acid glutamine (Gln; Q) and
ii. wherein amino acid asparagine (Asn; N) in position 30 with reference to SEQ ID NO: 40 is replaced by an amino acid selected from serine (Ser; S), threonine (Thr; T) and aspartate (Asp; D).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 40 or a conservative variant thereof or a sequence at least 90% identical thereto, and (b) a light chain variable region (VL) amino acid sequence comprising SEQ ID NO: 20 or a conservative variant thereof or a sequence at least 90% identical thereto, wherein
i. amino acid glutamate (Glu; E) in position 1 with reference to SEQ ID NO: 40 is replaced by amino acid glutamine (Gln; Q); and
ii. wherein amino acid asparagine (Asn; N) in position 30 with reference to SEQ ID NO: 40 is replaced by an amino acid selected from serine (Ser; S), threonine (Thr; T) and aspartate (Asp; D); and
iii. wherein amino acid methionine (Met; M) in position 54 with reference to SEQ ID NO: 40 is replaced by an amino acid selected from tyrosine (Tyr; Y), asparagine (Asn; N) and isoleucine (Ile; I).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid comprising SEQ ID NO: 40 or a conservative variant thereof or a sequence at least 90% identical thereto, and (b) a light chain variable region (VL) amino acid sequence comprising SEQ ID NO: 20 or a conservative variant thereof or a sequence at least 90% identical thereto, wherein
i. amino acid glutamate (Glu; E) in position 1 with reference to SEQ ID NO: 40 is replaced by amino acid glutamine (Gln; Q) and
ii. wherein amino acid serine (Ser; S) in position 31 with reference to SEQ ID NO: 40 is replaced by an amino acid selected from threonine (Thr; T), asparagine (Asn; N) and alanine (Ala; A).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising SEQ ID NO: 40 or a conservative variant thereof or a sequence at least 90% identical thereto, and (b) a light chain variable region (VL) amino acid sequence comprising SEQ ID NO: 20 or a conservative variant thereof or a sequence at least 90% identical thereto, wherein
i. amino acid glutamate (Glu; E) in position 1 with reference to SEQ ID NO: 40 is replaced by amino acid glutamine (Gln; Q); and
ii. wherein amino acid serine (Ser; S) in position 31 with reference to SEQ ID NO: 40 is replaced by an amino acid selected from threonine (Thr; T), asparagine (Asn; N) and alanine (Ala; A).
iii. wherein amino acid methionine (Met; M) in position 54 with reference to SEQ ID NO: 40 is replaced by an amino acid selected from tyrosine (Tyr; Y), asparagine (Asn; N) and isoleucine (Ile; I).

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence selected from the group consisting of SEQ ID NO: 22, SEQ ID NO: 25, SEQ ID NO: 28, SEQ ID NO: 31, SEQ ID NO: 34, a conservative variant thereof, and a sequence at least 90% identical thereto, (b) and a light chain variable region (VL) amino acid sequence selected from the group consisting of SEQ ID NO: 16, a conservative variant thereof, and a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a heavy chain variable region (VH) amino acid sequence comprising the sequence shown in SEQ ID NO: 37 or a conservative variant thereof or sequences at least 90% identical thereto, and (b) a light chain variable region (VL) amino acid sequence comprising the sequence shown in SEQ ID NO: 20 or a conservative variant thereof or sequences at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence selected from the group consisting of SEQ ID NOs: 43, 47, 50, 53, 56, 96, 100, 103, 116, 134, 142, 158, a conservative variant thereof, and a sequence at least 90% identical thereto; and (b) a VL amino acid sequence selected from the group consisting of SEQ ID NOs: 45, 98, 118, 136, 144, 150, 156, 160, a conservative variant thereof, and a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence comprising the sequence shown in SEQ ID NO: 43 or a conservative variant thereof or a sequence at least 90% identical thereto, and (b) a VL amino acid sequence comprising the sequence shown in SEQ ID NO: 45 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence comprising the sequence shown in SEQ ID NO: 158 or a conservative variant thereof or a sequence at least 90% identical thereto, and (b) a VL amino acid sequence comprising the sequence shown in SEQ ID NO: 160 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence selected from the group consisting of SEQ ID NO: 47, SEQ ID NO: 50, SEQ ID NO: 56, a conservative variant thereof, and a sequence at least 90% identical thereto, and (b) a VL amino acid sequence selected from the group consisting of SEQ ID NO: 45, a conservative variant thereof, and a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence selected from the group consisting of SEQ ID NO: 53, SEQ ID NO: 100, a conservative variant thereof, and a sequence at least 90% identical thereto, and (b) a VL amino acid sequence from the group consisting of SEQ ID NO: 160, a conservative variant thereof, and a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds I1-18, in particular that specifically binds IL-18 but does not bind the IL-18/IL-18 binding protein complex, comprises (a) a VH amino acid sequence selected from the group consisting of SEQ ID NO: 96, SEQ ID NO: 103, a conservative variant thereof, and a sequence at least 90% identical thereto, and (b) a VL amino acid sequence selected from the group consisting of SEQ ID NO: 98, a conservative variant thereof, and a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence comprising the sequence shown in SEQ ID NO: 116 or a conservative variant thereof or a sequence at least 90% identical, and (b) a VL amino acid sequence comprising the sequence shown in SEQ ID NO: 118 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence comprising the sequence shown in SEQ ID NO: 142 or a conservative variant thereof or a sequence at least 90% identical thereto, and (b) a VL amino acid sequence comprising the sequence shown in SEQ ID NO: 144 or a conservative variant thereof or a sequence at least 90% identical thereto.

In one embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises (a) a VH amino acid sequence comprising the sequence shown in SEQ ID NO: 134 or a conservative variant thereof or a sequence at least 90% identical thereto, and (b) a VL amino acid sequence comprising the sequences shown in SEQ ID NO: 136 or SEQ ID NO: 150 or SEQ ID NO: 156 or a conservative variant thereof or a sequence at least 90% identical thereto.

In one embodiment, the IL-18 antagonist of the disclosure, e.g., the anti-IL-18 antibody comprises a mutated or chemically modified amino acid Fc region, wherein the mutated or chemically modified amino acid Fc region prevents or decreases ADCC activity and/or increases half life when compared with a wild type Fc region. Preferably, the mutated or chemically modified amino acid Fc region is a silent IgG1 Fc region.

Also provided herein, the IL-18 antagonist, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, which has variable region heavy and light chain amino acid sequences or heavy and light chain amino acid sequences that are homologous to the amino acid sequences of the antibodies described herein, and wherein the homologous antibodies or fragment thereof retain the desired functional properties of the IL-18 antagonists according to the disclosure.

For example, the disclosure provides the IL-18 antagonist, e.g., the anti-IL-18 antibody or a fragment thereof, comprising a VH and a VL, wherein: the VH is at least 80%, or at least 90% identical to an amino acid sequence selected from the group consisting of SEQ ID NOs: 14; 18; 22; 25; 28; 31; 34; 37; 40; 83; 87; 90; 93; 112; 130 and 138; the VL is at least 80%, or at least 90% identical to an amino acid sequence selected from the group consisting of SEQ ID NOs: 16; 20; 85; 114; 132; 140; 147 and 153, in particular wherein the homologous antibody specifically binds to IL18 and does not bind the IL-18/IL-18 binding protein (IL-18 BP) complex. The homologous antibody may exhibit at least one additional functional properties such as inhibiting IL18 binding to IL18R or inhibiting IL18 dependent IFN-γ production.

In other embodiments, the VH and/or VL amino acid sequences may be 50%, 60%, 70%, 80%, 90%, 95%, 96%, 97%, 98% or 99% identical to the sequences set forth above. In other embodiments, the VH and/or VL amino acid sequences may be identical except an amino acid substitution in no more than 1, 2, 3, 4 or 5 amino acid positions. An IL-18 antagonist, e.g., an anti-IL-18 antibody or a fragment thereof, having VH and VL regions having high (i. e., 80% or greater) identity to the VH and VL regions of SEQ ID Nos: 14; 18; 22; 25; 28; 31; 34; 37; 40; 83; 87; 90; 93; 112; 130 or 138 and SEQ ID Nos: 16; 20; 85; 114; 132; 140; 147 or 153 respectively, can be obtained by mutagenesis (e.g., site-directed or PCR-mediated mutagenesis) of nucleic acid molecules encoding SEQ ID NOs: 15; 19; 23; 26; 29; 32; 35; 38; 41; 84; 88; 91; 94; 113; 131; 139; 146 or 152 and 17; 21; 24; 27; 30; 33; 36; 39; 42; 86; 89; 92; 95; 115; 133; 141; 148 or 154, respectively, followed by testing of the encoded altered antibody for retained function (i. e., the functions set forth above) using the functional assays described herein.

The homologous antibody can be, for example, a human antibody, a humanized antibody or a chimeric antibody. Preferably the antibody is a fully human silent IgG1 antibody.

In a specific embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises: a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8.

In a specific embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(a) a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8; and
(b) a mutation replacing the heavy chain framework amino acid asparagine 30 with lysine (N30K).

In a more specific embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(a) a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8; and
(b) a mutation replacing the heavy chain framework amino acid asparagine 30 with lysine (N30K); and
(c) a heavy chain variable domain comprising SEQ ID NO: 14 or a sequence at least 90% identical thereto; and a light chain variable domain comprising SEQ ID NO: 16 or a sequence at least 90% identical thereto.

In a specific embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises a heavy chain variable domain comprising SEQ ID NO: 14 and a light chain variable domain comprising SEQ ID NO: 16.

In a more specific embodiment, the IL-18 antagonist of the disclosure, e.g., the anti-IL-18 antibody or a fragment thereof, e.g., the anti-IL-18 antibody or the fragment thereof that specifically binds IL-18, in particular that specifically binds IL-18 but does not bind the IL-18/IL-18 binding protein complex, comprises:
(a) a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8; and
(b) a mutation replacing the heavy chain framework amino acid asparagine 30 with lysine (N30K); and
(c) a heavy chain comprising SEQ ID NO: 43 or a sequence at least 90% identical thereto; and a light chain comprising SEQ ID NO: 45 or a sequence at least 90% identical thereto.

In a specific embodiment, the IL-18 antagonist of the disclosure, e.g., the anti-IL-18 antibody or a fragment thereof, e.g., the anti-EL-18 antibody or the fragment thereof that specifically binds IL-18, in particular that specifically binds IL-18 but does not bind the IL-18/IL18 binding protein complex, comprises a heavy chain comprising SEQ ID NO: 43 and a light chain comprising SEQ ID NO: 45.

In a preferred embodiment, the anti-IL-18 antibody of the disclosure or the fragment thereof used in methods and uses of the invention is MOR9464_N30K antibody (also referred herein as CMK389, see Table 1 of the present disclosure) or a fragment thereof described in WO 2014/037899, the entire contents of which are hereby incorporated by reference.

TABLE 1
Sequences of IL-18 and CMK389 (MOR9464_N30K)

| SEQ | | |
|---|---|---|
| ID | Descrip- | |
| NO: | tion | Sequence |
| 1 | Human | MAAEPVEDNCINFVAMKFIDNTLYFIAEDDENLES |
| | IL-18 | DYFGKLESKLSVIRNLNDQVLFIDQGNRPLFEDMT |
| | | DSDCRDNAPRTIFIISMYKDSQPRGMAVTISVKCE |
| | | KISTLSCENKIISFKEMNPPDNIKDTKSDIIFFQR |
| | | SVPGHDNKMQFESSSYEGYFLACEKERDLFKLILK |
| | | KEDELGDRSIMFTVQNED |
| 2 | Cyno- | MAAEPAEDNCINEVAMKPIDSTLYFIAEDDENLES |
| | molgus | DYFGKLESKLSIIRNLNDQVLFIDQGNRPLFEDMT |
| | IL-18 | DSDCRDNAPRTIFIINMYKDSQPRGMAVAISVKCE |
| | | KISTLSCENRIISFKEMNPPDNIKDTKSDIIFFQR |
| | | SVPGHDNKMQFESSSYEGYFLACEKERDLYKLILK |
| | | KKDELGDRSIMFTVQNED |

CMK389 (MOR9464_N30K)

| | | |
|---|---|---|
| 3 | H-CDR1 | SYAIS |
| 9 | H-CDR2 | NIIPMTGQTYYAQKFQG |
| 5 | H-CDR3 | AAYHPLVEDN |
| 6 | L-CDR1 | SGSSSNIGNHYVN |
| 7 | L-CDR2 | RNNHRPS |
| 8 | L-CDR3 | QSWDYSGFSTV |
| 14 | VH | EVQLVQSGAEVKKPGSSVKVSCKASGGTFKSYAIS |
| | | WVRQAPGQGLEWMGNIIPMTGQTYYAQKFQGRVTI |
| | | TADESTSTAYMELSSLRSEDTAVYYCARAAYHPLV |
| | | EDNWGQGTLVTVSS |
| 15 | Poly- | GAGGTGCAGCTGGTGCAGTCAGGCGCCGAAGTGAA |
| | nucleo- | GAAACCCGGCTCTAGCGTGAAAGTCAGCTGTAAAG |
| | tide | CTAGTGGCGGCACCTTCAAGTCCTACGCTATTAGC |
| | VH | TGGGTCAGACAGGCCCCAGGTCAGGGCCTGGAGTG |
| | | GATGGGCAATATTATCCCTATGACCGGTCAGACCT |
| | | ACTACGCTCAGAAATTTCAGGGTAGAGTGACTATC |
| | | ACCGCCGACGAGTCTACTAGCACCGCCTATATGGA |
| | | ACTGTCTAGCCTGAGATCAGAGGACACCGCCGTCT |
| | | ACTACTGCGCTAGAGCCGCCTATCACCCCCTGGTG |
| | | TTCGATAACTGGGGTCAGGGCACCCTGGTCACCGT |
| | | GTCTAGC |
| 16 | VL | DIVLTQPPSVSGAPGQRVTISCSGSSSNIGNHYVN |
| | | WYQQLPGTAPKLLIYRNNHRPSGVPDRESGSKSGT |
| | | SASLAITGLQSEDEADYYCQSWDYSGFSTVFGGGT |
| | | KLTVL |
| 17 | Poly- | GATATCGTCCTGACTCAGCCCCCTAGCGTCAGCGG |
| | nucleo- | CGCTCCCGGTCAGAGAGTGACTATTAGCTGTAGCG |
| | tide | GCTCTAGCTCTAATATCGGTAATCACTACGTGAAC |
| | VL | TGGTATCAGCAGCTGCCCGGCACCGCCCCTAAGCT |
| | | GCTGATCTATAGAAACAATCACCGGCCTAGCGGCG |
| | | TGCCCGATAGGTTTAGCGGATCTAAGTCAGGCACT |
| | | AGCGCTAGTCTGGCTATCACCGGACTGCAGTCAGA |
| | | GGACGAGGCCGACTACTACTGTCAGTCCTGGGACT |
| | | ATAGCGGCTTTAGCACCGTGTTCGGCGGAGGCACT |
| | | AAGCTGACCGTGCTG |
| 43 | Heavy | EVQLVQSGAEVKKPGSSVKVSCKASGGTFKSYAIS |
| | chain | WVRQAPGQGLEWMGNIIPMTGQTYYAQKFQGRVTI |
| | | TADESTSTAYMELSSLRSEDTAVYYCARAAYHPLV |
| | | EDNWGQGTLVTVSSASTKGPSVFPLAPSSKSTSGG |
| | | TAALGCLVKDYFPEPVTVSWNSGALTSGVHTFPAV |
| | | LQSSGLYSLSSVVTVPSSSLGTQTYICNVNHKPSN |
| | | TKVDKRVEPKSCDKTHTCPPCPAPEAAGGPSVFLF |
| | | PPKPKDTLMISRTPEVTCVVVDVSHEDPEVKENWY |
| | | VDGVEVHNAKTKPREEQYNSTYRVVSVLTVLHQDW |
| | | LNGKEYKCKVSNKALPAPIEKTISKAKGQPREPQV |
| | | YTLPPSREEMTKNQVSLTCLVKGFYPSDIAVEWES |
| | | NGQPENNYKTTPPVLDSDGSFFLYSKLTVDKSRWQ |
| | | QGNVFSCSVMHEALHNHYTQKSLSLSPGK |
| 44 | Poly- | GAGGTGCAGCTGGTGCAGTCAGGCGCCGAAGTGAA |
| | nucleo- | GAAACCCGGCTCTAGCGTGAAAGTCAGCTGTAAAG |
| | tide | CTAGTGGCGGCACCTTCAAGTCCTACGCTATTAGC |
| | Heavy | TGGGTCAGACAGGCCCCAGGTCAGGGCCTGGAGTG |
| | chain | GATGGGCAATATTATCCCTATGACCGGTCAGACCT |
| | | ACTACGCTCAGAAATTTCAGGGTAGAGTGACTATC |
| | | ACCGCCGACGAGTCTACTAGCACCGCCTATATGGA |
| | | ACTGTCTAGCCTGAGATCAGAGGACACCGCCGTCT |
| | | ACTACTGCGCTAGAGCCGCCTATCACCCCCTGGTG |
| | | TTCGATAACTGGGGTCAGGGCACCCTGGTCACCGT |
| | | GTCTAGCGCTAGCACTAAGGGCCCCTCCGTGTTCC |
| | | CTCTGGCCCCTTCCAGCAAGTCTACCTCCGGCGGC |
| | | ACAGCTGCTCTGGGCTGCCTGGTCAAGGACTACTT |
| | | CCCTGAGCCTGTGACAGTGTCCTGGAACTCTGGCG |
| | | CCCTGACCTCTGGCGTGCACACCTTCCCTGCCGTG |
| | | CTGCAGTCCTCCGGCCTGTACTCCCTGTCCTCCGT |
| | | GGTCACAGTGCCTTCAAGCAGCCTGGGCACCCAGA |
| | | CCTATATCTGCAACGTGAACCACAAGCCTTCCAAC |
| | | ACCAAGGTGGACAAGCGGGTGGAGCCTAAGTCCTG |
| | | CGACAAGACCCACACCTGTCCTCCCTGCCCTGCTC |
| | | CTGAAGCTGCTGGCGGCCCTTCTGTGTTCCTGTTC |
| | | CCTCCAAAGCCCAAGGACACCCTGATGATCTCCCG |
| | | GACCCCTGAAGTGACCTGCGTGGTGGTGGACGTGT |
| | | CCCACGAGGATCCTGAAGTGAAGTTCAATTGGTAC |
| | | GTGGACGGCGTGGAGGTGCACAACGCCAAGACCAA |
| | | GCCTCGGGAGGAACAGTACAACTCCACCTACCGGG |
| | | TGGTGTCCGTGCTGACCGTGCTGCACCAGGACTGG |
| | | CTGAACGGCAAAGAGTACAAGTGCAAAGTCTCCAA |
| | | CAAGGCCCTGCCTGCCCCTATCGAAAAGACAATCT |
| | | CCAAGGCCAAGGGCCAGCCTAGGGAACCCCAGGTG |
| | | TACACCCTGCCACCCAGCCGGGAGGAAATGACCAA |
| | | GAACCAGGTGTCCCTGACCTGTCTGGTCAAGGGCT |
| | | TCTACCCTTCCGATATCGCCGTGGAGTGGGAGTCT |
| | | AACGGCCAGCCTGAGAACAACTACAAGACCACCCC |
| | | TCCTGTGCTGGACTCCGACGGCTCCTTCTTCCTGT |
| | | ACTCCAAACTGACCGTGGACAAGTCCCGGTGGCAG |
| | | CAGGGCAACGTGTTCTCCTGCTCCGTGATGCACGA |
| | | GGCCCTGCACAACCACTACACCCAGAAGTCCCTGT |
| | | CCCTGTCTCCCGGCAAG |
| 45 | Light | DIVLTQPPSVSGAPGQRVTISCSGSSSNIGNHYVN |
| | chain | WYQQLPGTAPKLLIYRNNHRPSGVPDRESGSKSGT |
| | | SASLAITGLQSEDEADYYCQSWDYSGFSTVFGGGT |
| | | KLTVLGQPKAAPSVTLFPPSSEELQANKATLVCLI |
| | | SDFYPGAVTVAWKADSSPVKAGVETTTPSKQSNNK |
| | | YAASSYLSLTPEQWKSHRSYSCQVTHEGSTVEKTV |
| | | APTECS |
| 46 | Poly- | GATATCGTCCTGACTCAGCCCCCTAGCGTCAGCGG |
| | nucleo- | CGCTCCCGGTCAGAGAGTGACTATTAGCTGTAGCG |
| | tide | GCTCTAGCTCTAATATCGGTAATCACTACGTGAAC |
| | Light | TGGTATCAGCAGCTGCCCGGCACCGCCCCTAAGCT |
| | chain | GCTGATCTATAGAAACAATCACCGGCCTAGCGGCG |
| | | TGCCCGATAGGTTTAGCGGATCTAAGTCAGGCACT |
| | | AGCGCTAGTCTGGCTATCACCGGACTGCAGTCAGA |
| | | GGACGAGGCCGACTACTACTGTCAGTCCTGGGACT |
| | | ATAGCGGCTTTAGCACCGTGTTCGGCGGAGGCACT |
| | | AAGCTGACCGTGCTGGGTCAGCCTAAGGCTGCCCC |
| | | CAGCGTGACCCTGTTCCCCCCCAGCAGCGAGGAGC |
| | | TGCAGGCCAACAAGGCCACCCTGGTGTGCCTGATC |
| | | AGCGACTTCTACCCAGGCGCCGTGACCGTGGCCTG |
| | | GAAGGCCGACAGCAGCCCCGTGAAGGCCGGCGTGG |
| | | AGACCACCACCCCCAGCAAGCAGAGCAACAACAAG |
| | | TACGCCGCCAGCAGCTACCTGAGCCTGACCCCCGA |
| | | GCAGTGGAAGAGCCACAGGTCCTACAGCTGCCAGG |
| | | TGACCCACGAGGGCAGCACCGTGGAAAAGACCGTG |
| | | GCCCCAACCGAGTGCAGC |
| 65 | (Chothia) | GGTFKSY |
| | H-CDR1 | |
| 60 | (Chothia) | IPMTGQ |
| | H-CDR2 | |
| 61 | (Chothia) | AAYHPLVEDN |
| | H-CDR3 | |
| 62 | (Chothia) | SSSNIGNHY |
| | L-CDR1 | |
| 63 | (Chothia) | RNN |
| | L-CDR2 | |
| 64 | (Chothia) | WDYSGEST |
| | L-CDR3 | |

### Biomarkers

According to other exemplary embodiments, the present disclosure provides methods for treating AD or a related condition in a subject, the methods comprising: (a) selecting a subject who exhibits an elevated level of at least one AD-associated biomarker; and (b) administering to the subject an IL-18 antagonist, e.g., an anti-IL-18 antibody or a fragment thereof, or a pharmaceutical composition comprising a therapeutically effective amount of an IL-18 antagonist, e.g., an anti-IL-18 antibody or a fragment thereof. Exemplary AD-associated biomarkers that can be evaluated and/or measured in the context of the present disclosure include, but are not limited to, thymus and activation-regulated chemokine (TARC; also known as CCL17), immunoglobulin E (IgE), eotaxin-3 (also known as CCL26), MDC (also known as CCL22), hsCRP (high-sensitivity C-reactive protein), IL-18, (e.g., serum IL-18, serum free IL-18 (bioactive)), IL-18BP (e.g., serum IL-18BP, serum free IL-18BP), lactate dehydrogenase (LDH), eosinophils, antigen-specific IgE (e.g., Phadiatop™ test), CD40, IL-24, IL-22, and periostin. In some embodiments, the methods of the present disclosure comprise determining the level of an AD-associated biomarker in a subject selecting a subject with an elevated level of the AD-associated biomarker, and administering a therapeutically effective amount of an IL-18 antagonist, e.g., an anti-IL-18 antibody or a fragment thereof. In some embodiments, the subject is selected by acquiring information about the level of an AD-associated biomarker in a subject. In some embodiments, the level of an AD-associated biomarker is determined by an assay or test known in the art. In one embodiment, the subject is selected on the basis of exhibiting an IgE level greater than about 1500 kU/L prior to or at the time of treatment. In one embodiment, the subject is selected on the basis of exhibiting a TARC level of greater than about 1000 pg/mL prior to or at the time of treatment. According to a related aspect of the present disclosure, methods for treating AD are provided which comprise administering to a subject a therapeutically effective amount of an IL-18 antagonist, e.g., an anti-IL-18 antibody or a fragment thereof, or a pharmaceutical composition comprising a therapeutically effective amount of an IL-18 antagonist, e.g., an anti-IL-18 antibody or a fragment thereof, wherein the administration to the subject results in a decrease in at least one AD-associated biomarker by day 4, 8, 15, 22, 25, 29, 36 or later in the subject following administration. In certain embodiments, the subject exhibits between 5% and 20% decrease in IgE level from the baseline after 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks or 16 weeks or later following administration. In certain embodiments, the subject exhibits between 25% and 70% decrease in TARC level from baseline after 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks or 16 weeks or later following administration.

According to other exemplary embodiments, the present disclosure provides methods for treating AD or a related condition in a subject, the methods comprising: (a) selecting a subject who exhibits an elevated level of IL-18 or IL-18BP; and (b) administering to the subject an IL-18 antagonist, e.g., an anti-IL-18 antibody or a fragment thereof, or a pharmaceutical composition comprising a therapeutically effective amount of an IL-18 antagonist, e.g., an anti-IL-18 antibody or a fragment thereof. In some embodiments, the methods of the present disclosure comprise determining the level of IL-18 or IL-18BP in a subject, selecting a subject with an elevated level of IL-18 or IL-18BP, and administering to the subject a therapeutically effective amount of an IL-18 antagonist, e.g., an anti-IL-18 antibody or a fragment thereof. In some embodiments, the subject is selected by acquiring information about the level of IL-18 or IL-18BP in a subject. In some embodiments, the level of IL-18 or IL-18BP is determined by an assay or test known in the art. According to a related aspect of the present disclosure, methods for treating AD are provided which comprise administering to a subject a therapeutically effective amount of an IL-18 antagonist, e.g., an anti-IL-18 antibody or a fragment thereof, or a pharmaceutical composition comprising a therapeutically effective amount of an IL-18 antagonist, e.g., an anti-IL-18 antibody or a fragment thereof, wherein the administration to the subject results in a decrease in IL-18 or IL-18BP level by day 4, 8, 15, 22, 25, 29, 36 or later in the subject following administration. Also provided herein are methods for decreasing the level of one or more AD-associated biomarker(s) in a subject, or improving one or more AD-associated parameter(s) in a subject, wherein the methods comprise sequentially administering to a subject in need thereof a single initial dose of an IL-18 antagonist, e.g., an anti-IL-18 antibody or a fragment thereof, or a pharmaceutical composition comprising an IL-18 antagonist, e.g., an anti-IL-18 antibody or a fragment thereof, followed by one or more secondary doses of an IL-18 antagonist, e.g., an anti-IL-18 antibody or a fragment thereof, or a pharmaceutical composition comprising an IL-18 antagonist, e.g., an anti-IL-18 antibody or a fragment thereof.

Also provided herein are methods of monitoring the effectiveness of treatment of AD, in particular of moderate-to-severe AD, or a related condition in a subject with an IL-18 antagonist, e.g., an anti-IL-18 antibody or a fragment thereof, the method comprising:
(a) determining the expression level of an AD-associated biomarker, such as CCL17/TARC, IgE (e.g., serum IgE), CCL26/eotaxin-3, CCL22/MDC, hsCRP, IL-18 (e.g., serum IL-18, serum free IL-18 (bioactive)), IL-18BP (e.g., serum IL-18BP, e.g., serum free IL-18BP), CD40, IL-24, IL-22 in a biological sample acquired from the subject before treatment with the IL-18 antagonist, e.g., an anti-IL-18 antibody or a fragment thereof;
(b) determining the expression level of the same AD-associated biomarker(s) as step (a), in a biological sample acquired from the subject after treatment with the IL-18 antagonist, e.g., an anti-IL-18 antibody or a fragment thereof;
(c) comparing the level determined in step (a) with the level in step (b); and (d) concluding that the treatment is effective when the level determined in step (b) is lower than the level determined in step (a), or concluding that the treatment is not effective when the level determined in step (b) is the same or higher than the level determined in step (a).

In one embodiment, the level in step (b) is determined 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks or 16 weeks after determining the level in step (a). In one embodiment, the biomarker is TARC, and if TARC level decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. In one embodiment, the biomarker is IgE, and if IgE level decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. In one embodiment, the biomarker is eotaxin-3, and if eotaxin-3 level decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. In one embodiment, the biomarker is CCL22/MDC, and if CCL22/MDC level decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. In one embodiment, the biomarker is IL-18 (e.g., serum IL-18, serum free IL-18 (bioactive)), and if IL-18 level (e.g., serum IL-18 level, e.g., level of serum free IL-18 (bioactive)) decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. In one embodiment, the biomarker is IL-18BP, and if IL-18BP level decreases following administration of the IL-18 antagonist, e.g., the anti-IL-18 antibody or a fragment thereof, then treatment with the IL-18 antagonist, e.g., the anti-IL-18 antibody or a fragment thereof, is determined to be effective. The expression level of the biomarker can be determined, for example, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks or longer after administration of the IL-18 antagonist, e.g., the anti-IL-18 antibody or a fragment thereof, and compared to the expression level prior to administration of the IL-18 antagonist, e.g., the anti-IL-18 antibody or a fragment thereof. The dose or the dosing regimen of the IL-18 antagonist, e.g., the anti-IL-18 antibody or a fragment thereof, can be adjusted following the determination. For example, if the expression of the biomarker fails to decrease within 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks or longer following administration of the IL-18 antagonist, e.g., the anti-IL-18 antibody or a fragment thereof, then treatment with the IL-18 antagonist, e.g., the anti-IL-18 antibody or a fragment thereof, can be stopped, or the dose of the IL-18 antagonist, e.g., the anti-IL-18 antibody or a fragment thereof, can be increased. If expression of the biomarker decreases following administration of the IL-18 antagonist, e.g., the anti-IL-18 antibody or a fragment thereof, the dosage of the I1-18 antagonist e.g., the anti-IL-18 antibody or a fragment thereof, can be maintained or decreased, such as to identify a minimal effective dose. In some embodiments, treatment is maintained at the minimal effective dose.

Also, provided herein are methods for monitoring a subject's response to treatment with an IL-18 antagonist, e.g., an anti-IL-18 antibody or a fragment thereof, wherein the subject has AD, in particular moderate-to-severe AD, or a related condition, the method comprising: (a) acquiring information regarding the expression level of one or more AD-associated biomarker, in particular one or more AD-associated biomarker selected from the list consisting of CCL17/TARC, IgE (e.g., serum IgE), CCL26/eotaxin-3, CCL22/MDC, hsCRP, CD40, IL-24, IL-22, IL-18 (e.g., serum IL-18, serum free IL-18 (bioactive)), and IL-18BP (e.g., serum IL-18BP) in a biological sample from the subject following administration of the IL-18 antagonist, e.g., the anti-IL-18 antibody or a fragment thereof, to the subject; and (b) providing an indication that the treatment should be continued if the expression level of one or more AD-associated biomarker, in particular one or more AD-associated biomarker selected from the list consisting of CCL17/TARC, IgE (e.g., serum IgE), CCL26/eotaxin-3, CCL22/MDC, hsCRP, CD40, I1-24, I1-22, IL-18 (e.g., serum I1-18, serum free I1-18 (bioactive)), and IL-18BP (e.g., serum IL-18BP), has decreased as compared to the level before treatment with the IL-18 antagonist, e.g., the anti-IL-18 antibody or a fragment thereof. In one embodiment, the biomarker is TARC, and if TARC level is determined to decrease following administration of the IL-18 antagonist, e.g., the anti-IL-18 antibody or a fragment thereof, then an indication is provided to continue treatment with the IL-18 antagonist, e.g., the anti-IL-18 antibody or a fragment thereof. In one embodiment, the biomarker is IgE, and if IgE level is determined to decrease following administration of the IL-18 antagonist, e.g., the anti-IL-18 antibody or a fragment thereof, then an indication is provided to continue treatment with the IL-18 antagonist, e.g., the anti-IL-18 antibody or a fragment thereof.

### Resistant, Non-Responsive or Inadequately Responsive Subjects

In certain embodiments provided herein are methods to treat a subject that is resistant, non-responsive or inadequately responsive to treatment with a topical AD therapy, e.g., with a topical corticosteroid (TCS) or a calcineurin inhibitor. In certain embodiments provided herein are methods to treat a subject that is refractory to topical AD therapy or a subject that did not adequately respond to treatment with a topical AD therapy, e.g., with a topical corticosteroid (TCS) or a calcineurin inhibitor.

As used herein, the phrases “inadequately controlled”, “inadequate response”, “did not adequately respond” and the like refer to treatments that produce an insufficient response or treatment failure in a subject, e.g., following treatment with a given agent a subject still has one or more pathological signs and symptoms of the disorder, e.g., in the case of AD, symptoms include intense pruritus (e.g., severe itch) and sleep disorders due to the itch. Signs include scaly and dry, often erythematous, lesions, possibly accompanied with local or wider spread edema, vesiculation with itchy small skin blisters called vesicles, and oozing and/or weeping in the acute stage, while erosions and skin harder and thickened areas (often as well called lichenification due to repeated rubbing and scratching) are signs of chronic stage of dermatitis. In some embodiments, prior to administering the IL-18 antagonist, e.g., an anti-IL-18 antibody or a fragment thereof, the subject has had an inadequate response to prior treatment with an atopic dermatitis therapy. In some embodiments, the subject has had an inadequate response to prior treatment with a topical AD therapy, e.g., a topical steroid (e.g., a topical corticosteroid). A subject who has responded adequately to treatment with an atopic dermatitis therapy (a topical AD therapy, e.g., a topical steroid, e.g., a topical corticosteroid), but has discontinued due to a side effect is termed “intolerant”. In some embodiments, the subject having AD or a related condition to be treated using the disclosed methods, uses, kits, etc., is intolerant to a prior AD therapy (a topical AD therapy, e.g., a topical steroid, e.g., a topical corticosteroid). In some embodiments, the subject is refractory to topical corticosteroid therapy. In some embodiments, the subject did not adequately respond to treatment with topical corticosteroid therapy.

Refractory refers to a particular type of inadequate response, i.e., by “refractory” is meant that the subject has been treated with at least 4 weeks of high potency AD therapy (a topical AD therapy, e.g., a topical steroid, e.g., a topical corticosteroid) without significant improvement. In some embodiments, the subject having AD or related condition who is treated according to the disclosed methods, uses, kits, etc., is refractory to treatment with a prior AD therapy (a topical AD therapy, e.g., a topical steroid, e.g., a topical corticosteroid). In some embodiments, the subject is refractory to topical corticosteroid therapy. In some embodiments, the subject did not adequately respond to treatment with topical corticosteroid therapy.

As used herein, “AD therapy” or “atopic dermatitis therapy” refers to atopic dermatitis treatments employing atopic dermatitis agents (e.g., small molecules, biological therapies.) or employing an atopic dermatitis modality (e.g., phototherapy), including topical therapies, systemic therapies, phototherapies, and combinations thereof. “AD therapy” includes topical therapies. “Topical AD therapy” or “topical atopic dermatitis therapy” in particular refers to AD therapy in the form of creams, ointments, lotions, gels or sprays (e.g., low-medium potency corticosteroids [Group IV-VII according to WHO guidelines, see Bolognia J L, Jorizzo J L, Schaffer J V. Glucocorticosteroids. Dermatology. 3rd ed. 2012. Ch 125, 2075-88; Ference J D, Last A R. Choosing topical corticosteroids. Am Fam Physician. 2009 Jan. 15; 79(2):135-40]); over the counter (OTC) emollients, and medical devices or so called barrier creams (such as atopiclair); and lubricants for the treatment of itch and/or pain, e.g. anti-itch lotions containing menthol, pramoxine or anti-histamines; local anesthetics, systemic agents (e.g., biological agents, e.g., IL-4R inhibitors, such as dupilumab; IL-13 inhibitors, such as tralokinumab and lebrikizumab; IL-13Ra1 inhibitors, such as ASLAN-004; IL-13Ra2 inhibitors; IL-31 inhibitors, such as nemolizumab; TNF alpha inhibitors, such as adalimumab, infliximab, certolizumab and etanercept, alefacept; IL-la inhibitors, such as bermekimab (MABp1); IL-23 inhibitors, such as briakinumab, ustekinumab, guselkumab, risankizumab, tildrakizumab; IL-17 inhibitors, such as brodalumab, ixekizumab; CD11a inhibitors, such as efalizumab; IL-22 inhibitors, such as fezalimumab, IL-22 binding proteins; IL-5 inhibitors, such as mepolizumab, benralizumab; a synthetic form of IL-2, such as aldesleukin; recombinant IL-2 approaches targeting the interleukin-2 receptor complex, such as LY3471851; OSMR inhibitors, such as KPL-716; VAP-1 inhibitors; OX-40 inhibitors or OX40L inhibitors such as GBR830, KY1005; IgE inhibitors, such as omalizumab, ligelizumab; TSLP inhibitors, such as tezepelumab; IL-33 inhibitors, such as MEDI3506; IL-36 inhibitors, such as spesolimab, ANB019; B-cell modulating approaches, such as rituximab, ocrelizumab; non-biological immunomodulating treatments, e.g., cyclosporine and other calcineurin inhibitors, JAK inhibitors such as tofacitinib, upadacitinib, abrocitinib, baricitinib; TYK2 inhibitors such as deucravacitinib; methotrexate; PDE4 inhibitors such as apremilast; Siglec inhibitor such as AK-002; S1P agonists or antagonists, such as etrasimod or SCD-044; BTK inhibitors such as TAS-5315, IRAK4 antagonists and CCR4-inhibiting approaches, such as RPT-193; systemic corticosteroids, cyclophosphamide, sulphasalazine, azathioprin, mycophenolate mofetil, dapson, hydroxychloroquine); retinoids (e.g., alitretinoin); leukotriene inhibitors or antileukotrienes, such as montelukast, pranlukast or zafirlukast, as well as 5-LO inhibitors such as zileuton, and LTA4H inhibitors such as acebilustat, intralesional corticosteroid injections; phototherapy (e.g. UVB and UVA high dose). photochemotherapy (e.g. psoralen and UVA (PUVA)); topical calcineurin inhibitors (cyclosporine, tacrolimus, pimecrolimus) or topical PDE4 inhibitors such as crisaborole, difamilast or roflumilast; topical JAK inhibitors such as ruxolitinib, delgocitinib, or topical Vitamin D analogues and topical aryl hydrocarbon receptor (AhR) inhibitors such as benvitimod/tapinarof, topical corticosteroids of high-ultrahigh potency (Group I, II, III as per WHO definition); anti-fungal drugs with known anti-inflammatory properties, e.g., griseofulvin, itraconazole, betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, Lactobacilli reuteri), omega-3, prednisone, prednisolone, platelet rich plasma, orabase paste, lycopene, topical chamomile, green tea, CO2 laser treatment, allergen specific immunotherapies, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, Chamaemelum nobile, sirolimus, tacrolimus, qingxuan decoction, NSAID topical rinse, NSAIDs, quercetin, NAVS naphthalan, valchlor, bupivacaine, oatmeal baths. Suitably, topical AD therapy is an atopic dermatitis prescription therapy including but not limited to topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab. In specific embodiments, topical AD therapy is a topical corticosteroid, in particular a low-medium potency topical corticosteroid. In certain embodiments, the topical corticosteroid (TCS) is selected from the group consisting of a group I TCS, a group II TCS and a group III TCS. In some embodiments, the TCS is selected from the group consisting of methylprednisolone aceponate, mometasone furoate, fluticasone propionate, betamethasone valerate and hydrocortisone butyrate. Preferred low-medium potency topical corticosteroids are Desoximetasone Cream, 0.05%, Fluocinolone acetonide Ointment, 0.025%, Fludroxycortide Ointment, 0.05%, Hydrocortisone valerate Ointment, 0.2%, Triamcinolone acetonide Cream, 0.1%, Betamethasone dipropionate Lotion, 0.02%, Betamethasone valerate Cream, 0.1%, Fluocinolone acetonide Cream, 0.025%, Fludroxycortide Cream, 0.05%, Hydrocortisone butyrate Cream, 0.1%, Hydrocortisone valerate Cream, 0.2%, Triamcinolone acetonide Lotion, 0.1%, Betamethasone valerate Lotion, 0.05%, Desonide Cream, 0.05%, Fluocinolone acetonide Solution, 0.01%, Dexamethasone sodium phosphate Cream, 0.1%, Hydrocortisone acetate Cream, 1%, Methylprednisolone acetate Cream, 0.25%.

In related embodiments, provided herein are methods to reduce the dependence on TCS in a subject with AD, e.g., moderate-to-severe AD, or related condition comprising concomitant administration of an IL-18 antagonist, e.g., an anti-IL-18 antibody or a fragment thereof, and a TCS, wherein the dosage of the TCS is reduced by 50% as compared to subjects without the administration of the IL-18 antagonist, e.g., the anti-IL-18 antibody or a fragment thereof. In one embodiment, provided herein are methods to reduce the dosage of a TCS in treatment of AD, e.g., moderate-to-severe AD, or a related condition, comprising administration of an IL-18 antagonist, e.g., an anti-IL-18 antibody or a fragment thereof, concomitantly with a reduced dosage of the TCS. The dosage of the TCS may be reduced by more than, for example, 10%, 20%, 30%, 40%, or 50%. In one embodiment, the dosage of the TCS may be reduced by more than, for example, 10%, 20%, 30%, 40%, or 50% as compared to the dosage used by the subject before treatment with the IL-18 antagonist, e e.g., the anti-IL-18 antibody or a fragment thereof.

### Administration and Concomitant Treatment

According to certain exemplary embodiments, the uses and methods of the present disclosure comprise administering the IL-18 antagonist, e.g., the anti-IL-18 antibody or a fragment thereof, subcutaneously, intravenously, intra-articularly or intra-spinally. In specific embodiments, the IL-18 antagonist, e.g., the anti-IL-18 antibody or a fragment thereof, is administered subcutaneously or intravenously.

Suitably, the uses and methods of the present disclosure comprise administering the IL-18 antagonist, e.g., the anti-IL-18 antibody or a fragment thereof, at a dose sufficient to achieve a therapeutically effective serum level. Suitably, the therapeutically effective serum level of the IL-18 antagonist, e.g., the anti-IL-18 antibody or a fragment thereof, is maintained during the treatment course.

As used herein, the terms “therapeutically effective serum level” refers to a serum level of a therapy in a subject (e.g., an IL-18 antagonist, e.g., anti-IL-18 antibody or a fragment thereof, e.g., CMK389) which is sufficient to reduce and/or ameliorate the severity and/or duration of a given condition, disorder, or disease and/or a symptom related thereto. In some aspects, “therapeutically effective serum level” as used herein also refers to the amount of an antagonist in serum of a subject which achieves a specified result, for example, improvements in AD-associated parameters, e.g., a decrease in Investigator's Global Assessment (IGA) score; a decrease from baseline in Dermatology Life Quality Index (DLQI); a decrease from baseline in a patient global impression of severity (PGIS); improvement (e.g. decrease from baseline) in a patient global impression of change (PGIC); a decrease in Body Surface Area Involvement of Atopic Dermatitis (BSA) score; a decrease in Eczema Area and Severity Index (EASI) score; a decrease in SCORAD score; and/or a decrease in Pruritus Numeric Rating Scale (NRS) score. In some aspects, “therapeutically effective serum level” as used herein also refers to the amount of an antagonist in serum of a subject which achieves a specified result, for example, decrease of the expression level of one or more AD-associated biomarker, in particular one or more AD-associated biomarker selected from the list consisting of CCL17/TARC, IgE (e.g., serum IgE), CCL26/eotaxin-3, CCL22/MDC, hsCRP, CD40, IL-24, IL-22, IL-18 (e.g., serum IL-18, serum free IL-18 (bioactive)), and IL-18BP (e.g., serum IL-18BP), as compared to the level before treatment with the IL-18 antagonist, e.g., the anti-IL-18 antibody or a fragment thereof.

Suitably, the uses and methods of the present disclosure comprise administering the IL-18 antagonist, e.g., the anti-IL-18 antibody or a fragment thereof, once a week, once every two weeks, once every three weeks, once every four weeks, once every eight weeks, or once every 12 weeks. According to certain exemplary embodiments, the uses and methods of the present disclosure comprise administering the IL-18 antagonist, e.g., the anti-IL-18 antibody or a fragment thereof, once every 4 weeks.

According to certain exemplary embodiments, the uses and methods of the present disclosure comprise administering an IL-18 antagonist, e.g., an anti-IL-18 antibody or a fragment thereof, and a second therapeutic agent. Suitably, the second therapeutic agent is administered to the subject before, after, or concurrent with the IL-18 antagonist, e.g., the anti-IL-18 antibody or a fragment thereof. Suitably, the second therapeutic agent is an AD agent, e.g., small molecule, biological therapy, or an agent employing AD modality, e.g., phototherapy, including topical therapy, systemic therapy, phototherapy, and combinations thereof. “AD agent” includes topical therapies in the form of creams, ointments, lotions, gels or sprays (e.g., low-medium potency corticosteroids [Group IV-VII according to WHO guidelines, see Bolognia J L, Jorizzo J L, Schaffer J V. Glucocorticosteroids. Dermatology. 3rd ed. 2012. Ch 125, 2075-88; Ference J D, Last A R. Choosing topical corticosteroids. Am Fam Physician. 2009 Jan. 15; 79(2):135-40]); over the counter (OTC) emollients, and medical devices or so called barrier creams (such as atopiclair); and lubricants for the treatment of itch and/or pain, e.g. anti-itch lotions containing menthol, pramoxine or anti-histamines; local anesthetics, systemic agents (e.g., biological agents, e.g., IL-4R inhibitors, such as dupilumab; IL-13 inhibitors, such as tralokinumab and lebrikizumab; IL-13Ra1 inhibitors, such as ASLAN-004; IL-13Ra2 inhibitors; IL-31 inhibitors, such as nemolizumab; TNF alpha inhibitors, such as adalimumab, infliximab, certolizumab and etanercept, alefacept; IL-la inhibitors, such as bermekimab (MABp1); IL-23 inhibitors, such as briakinumab, ustekinumab, guselkumab, risankizumab, tildrakizumab; IL-17 inhibitors, such as brodalumab, ixekizumab; CD11a inhibitors, such as efalizumab; IL-22 inhibitors, such as fezalimumab, IL-22 binding proteins; IL-5 inhibitors, such as mepolizumab, benralizumab; a synthetic form of IL-2, such as aldesleukin; recombinant IL-2 approaches targeting the interleukin-2 receptor complex, such as LY3471851; OSMR inhibitors, such as KPL-716; VAP-1 inhibitors; OX-40 inhibitors or OX40L inhibitors such as GBR830, KY1005; IgE inhibitors, such as omalizumab, ligelizumab; TSLP inhibitors, such as tezepelumab; IL-33 inhibitors, such as MEDI3506; IL-36 inhibitors, such as spesolimab, ANB019; B-cell modulating approaches, such as rituximab, ocrelizumab; non-biological immunomodulating treatments, e.g., cyclosporine and other calcineurin inhibitors, JAK inhibitors such as tofacitinib, upadacitinib, abrocitinib, baricitinib; TYK2 inhibitors such as deucravacitinib; methotrexate; PDE4 inhibitors such as apremilast; Siglec inhibitor such as AK-002; S1P agonists or antagonists, such as etrasimod or SCD-044; BTK inhibitors such as TAS-5315, IRAK4 antagonists and CCR4-inhibiting approaches, such as RPT-193; systemic corticosteroids, cyclophosphamide, sulphasalazine, azathioprin, mycophenolate mofetil, dapson, hydroxychloroquine); retinoids (e.g., alitretinoin); leukotriene inhibitors or antileukotrienes, such as montelukast, pranlukast or zafirlukast, as well as 5-LO inhibitors such as zileuton, and LTA4H inhibitors such as acebilustat, intralesional corticosteroid injections; phototherapy (e.g. UVB and UVA high dose). photochemotherapy (e.g. psoralen and UVA (PUVA)); topical calcineurin inhibitors (cyclosporine, tacrolimus, pimecrolimus) or topical PDE4 inhibitors such as crisaborole, difamilast or roflumilast; topical JAK inhibitors such as ruxolitinib, delgocitinib, or topical Vitamin D analogues and topical aryl hydrocarbon receptor (AhR) inhibitors such as benvitimod/tapinarof; topical corticosteroids of high-ultrahigh potency (Group I, II, III as per WHO definition); anti-fungal drugs with known anti-inflammatory properties, e.g., griseofulvin, itraconazole, betamethasone, dexamethasone, INCB018424, triamcinolone, apremilast, turmeric past, glucosamine sulfate, triamcinolone acetonide, sesame oil, betamethasone dipropionate, clobetasol propionate, probiotics (e.g., Bifidobacterium animalis subst. lactis HN019, Lactobacilli reuteri), omega-3, prednisone, prednisolone, platelet rich plasma, orabase paste, lycopene, topical chamomile, green tea, CO2 laser treatment, allergen specific immunotherapies, polybiotics, photobiomodulation, metronidazole, doxycycline, minocycline, cedar honey, purslane, curcuminoids, alefacept, hexaminolevulinate, hydroxychloroquine, adcortyl, efalizumab, fluocinolone, co-enzyme Q10 mucoadhesive tablets, Chamaemelum nobile, sirolimus, tacrolimus, qingxuan decoction, NSAID topical rinse, NSAIDs, quercetin, NAVS naphthalan, valchlor, bupivacaine, oatmeal baths. Suitably, topical AD therapy is an atopic dermatitis prescription therapy including but not limited to topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab.

In specific embodiments, topical AD therapy is a topical corticosteroid, in particular a low-medium potency topical corticosteroid. In certain embodiments, the second therapeutic agent is a low to medium potency steroid, e.g., topical or oral steroid, e.g., corticosteroid. According to certain exemplary embodiments, the second therapeutic agent is selected from the group consisting of a group I topical corticosteroid (TCS), group II topical corticosteroid (TCS) and group III topical corticosteroid (TCS). In some embodiments, the TCS is selected from the group consisting of methylprednisolone aceponate, mometasone furoate, fluticasone propionate, betamethasone valerate and hydrocortisone butyrate. Preferred low-medium potency topical corticosteroids are Desoximetasone Cream, 0.05%, Fluocinolone acetonide Ointment, 0.025%, Fludroxycortide Ointment, 0.05%, Hydrocortisone valerate Ointment, 0.2%, Triamcinolone acetonide Cream, 0.1%, Betamethasone dipropionate Lotion, 0.02%, Betamethasone valerate Cream, 0.1%, Fluocinolone acetonide Cream, 0.025%, Fludroxycortide Cream, 0.05%, Hydrocortisone butyrate Cream, 0.1%, Hydrocortisone valerate Cream, 0.2%, Triamcinolone acetonide Lotion, 0.1%, Betamethasone valerate Lotion, 0.05%, Desonide Cream, 0.05%, Fluocinolone acetonide Solution, 0.01%, Dexamethasone sodium phosphate Cream, 0.1%, Hydrocortisone acetate Cream, 1%, Methylprednisolone acetate Cream, 0.25%. According to certain exemplary embodiments, the second therapeutic agent is selected from the group consisting of steroid, cyclosporine, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab.

### Pharmaceutical Composition

The uses and methods of the present disclosure comprise administering to a subject in need thereof a pharmaceutical composition comprising a therapeutically effective amount of an IL-18 antagonist, e.g., an anti-IL-18 antibody or a fragment thereof. In certain aspects, the present disclosure provides a composition comprising an IL-18 antagonist, e.g., an anti-IL-18 antibody or a fragment thereof, and useful for uses and methods of the present disclosure in the treatment of atopic dermatitis or related condition, the composition further comprising one or more pharmaceutically acceptable carriers and/or diluents.

The compositions provided herein are preferably pharmaceutical compositions comprising an IL-18 antagonist, e.g., an anti-IL-18 antibody or a fragment thereof, and a pharmaceutically acceptable carrier, diluent or excipient, for uses and methods of the present disclosure in the treatment of atopic dermatitis or a related condition. Such carriers, diluents and excipients are well known in the art, and the skilled artisan will find a formulation and a route of administration best suited to treat a subject with an IL-18 antagonist, e.g., an anti-IL-18 antibody or a fragment thereof of the present disclosure.

In one aspect provided herein is a pharmaceutical composition comprising an IL-18 antagonist, e.g., an anti-IL-18 antibody or a fragment thereof, for use in the treatment and/or prevention of AD or a related condition. In a certain embodiment provided herein is a pharmaceutical composition comprising an IL-18 antagonist, e.g., an anti-IL-18 antibody or a fragment thereof, for use in improving one or more AD-associated parameters in a subject in need thereof. In another embodiment the pharmaceutical composition comprises an IL-18 antagonist, e.g., an anti-IL-18 antibody or a fragment thereof, for use in the treatment of AD in a subject having an elevated level of a biomarker selected from the group consisting of CCL17/TARC, IgE, CCL26/eotaxin-3, CCL22/MDC, hsCRP, CD40, IL-24, IL-22, IL-18 (e.g., serum IL-18, serum free IL-18 (bioactive)), IL-18BP (e.g., serum IL-18BP, e.g., serum free Il-18BP), and periostin.

In certain embodiments, the pharmaceutical composition is administered to the subject before, after or concurrent with a second therapeutic agent. In some embodiments, the second therapeutic agent is a topical corticosteroid (TCS) or a calcineurin inhibitor.

The phrase “pharmaceutically acceptable” refers to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. The phrase “pharmaceutically acceptable formulation” or “pharmaceutical formulation” refers to a formulation consisting of those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio.

The following Examples illustrates the invention described above, but is not, however, intended to limit the scope of the invention in any way. Other test models known as such to the person skilled in the pertinent art can also determine the beneficial effects of the claimed invention.

### EXAMPLES

### Example 1: IL-18 Detection

IL-18 levels were measured in ex vivo cultured skin (FIG. 1).

Samples: Four mm lesional and non-lesional biopsies from ten AD patients were obtained, cut into 2 or 3 fragments and cultured for 24 h in 80 μl of medium. Skin from five healthy people undergoing abdominal surgeries were obtained, 4 mm biopsies were taken, split in half and cultured for 24 h in 80 μl of medium. Skin was cultured in INDM medium (Gibco; Cat.no. #21056-023) plus 10% KnockOut™ Serum Replacement (KO serum; Gibco; Cat.no. #10828010) plus 1% Penicillin-Streptomycin (P/S; Gibco; Cat.no. #15140122) at 37° C., 5% CO2. Culture supernatants were subsequently analyzed using 10-spot U-PLEX assays (Meso Scale Discovery assays from Meso Scale Diagnostics, MSD) including IL-18 (total IL-18) using a Sector Imager S600 Reader (MSD). Remaining samples were stored at −80° C. and used later for the detection of IL-18 by an ELISA that specifically detects active IL-18 (MBL International, Cat.no. #7620). Samples were diluted 1:10 or 1:20 for the ELISA. All measured concentrations were normalized to the weight of the corresponding biopsy fragment and are expressed as “pg/mL per mg”, as shown in Figures IA and B.

Samples: Four mm lesional biopsies from 8 AD patients were cut into 4 fragments and each biopsy fragment was cultured for 24 h in 100 μl IMDM medium (Gibco; Cat.no. #21056-023) plus 10% KnockOut™ Serum Replacement (KO serum; Gibco; Cat.no. #10828010) plus 1% Penicillin-Streptomycin (P/S; Gibco; Cat.no. #15140122) at 37° C., 5% CO2. Skin from seven healthy people undergoing abdominal or breast surgeries (5×female, 2×male) were obtained. Four mm biopsies were split in half and each fragment was cultured for 24 h (same conditions as for AD biopsies). Samples were stored at −80° C. until detection of active IL-18 by ELISA (MBL International, Cat.no. #7620). All measured concentrations were normalized to the weight of the corresponding biopsy fragment and are expressed as “pg/mL per mg”, as shown in FIG. 1C.

Results: IL-18 has been found to be upregulated in lesional biopsies from AD patients (FIG. 1).

### Example 2: CMK389 Treatment of Human AD Biopsies

Samples: 4 mm biopsies from 10 AD patients were cut into 4 fragments and cultured for 24 h in 100 μl of medium with or without CMK389 (at 150 μg/mL final concentration). Skin was cultured in IMDM medium (Gibco; Cat.no. #21056-023) plus 10% KnockOut™ Serum Replacement (KO serum; Gibco; Cat.no. #10828010) plus 1% Penicillin-Streptomycin (P/S; Gibco; Cat.no. #15140122) at 37° C., 5% CO2. Skin from 8 healthy volunteers undergoing abdominal surgery was obtained, 4 mm biopsies were split in half and cultured for 24 h in the same medium used for AD biopsies. Supernatants were centrifuged at low speed to remove cells in the supernatant without disrupting them and stored at −80° C. until analysis.

Olink (CD40, IL-24,): One microliter per sample of supernatant from samples described above was analyzed with the Inflammation panel from Olink Proteomics (92 analytes; cat no. #95302) using the BioMark™ HD real-time PCR platform (Fluidigm). Quality control of Olink chip data was carried out using their standard quality control pipeline (QC) in the Olink NPX Manager Software. Protein expression was normalized to the weight of each corresponding biopsy fragment and is shown as “relative protein expression per mg”. Remaining samples were stored at −80° C. and used later for the detection via MSD.

MSD (IL-22, CCL17/TARC): 25 microliter per sample of supernatant from samples described above were analyzed using 10-spot U-PLEX assays (Meso Scale Discovery assays from Meso Scale Diagnostics, MSD) including IL-22 and TARC (CCL17) on a Sector Imager S600 Reader (MSD). Measured concentrations were normalized to the weight of the corresponding biopsy fragment and are expressed as “pg/mL per mg”.

### Results:

FIGS. 2A-B represent relative levels (per mg biopsy) of soluble CD40 in supernatants of ex vivo untreated cultured skin of healthy people and atopic dermatitis patients (NL=non-lesional, L=lesional). FIG. 2C represents relative levels (per mg biopsy) of soluble CD40 in supernatants of ex vivo cultured skin of atopic dermatitis patients in absence (untreated) or presence of CMK389.

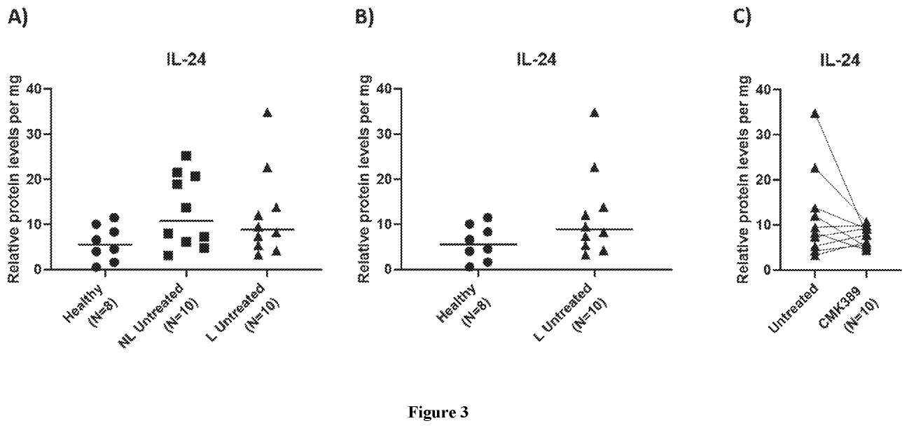

FIGS. 3A-B represent relative levels (per mg biopsy) of IL-24 in supernatants of ex vivo untreated cultured skin of healthy people and atopic dermatitis patients (NL=non-lesional, L=lesional). FIG. 3C represents relative levels (per mg biopsy) of IL-24 in supernatants of ex vivo cultured skin of atopic dermatitis patients in absence (untreated) or presence of CMK389.

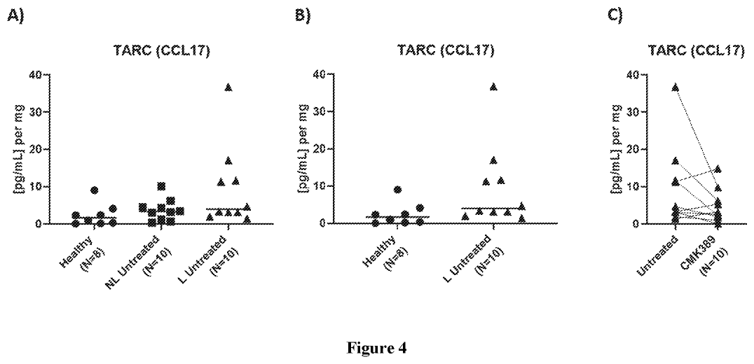

FIG. 4A-B represent relative levels (per mg biopsy) of TARC/CCL17 in supernatants of ex vivo untreated cultured skin of healthy people and atopic dermatitis patients (NL=non-lesional, L=lesional). FIG. 4C represents relative levels (per mg biopsy) of TARC/CCL17 in supernatants of ex vivo cultured skin of atopic dermatitis patients in absence (untreated) or presence of CMK389.

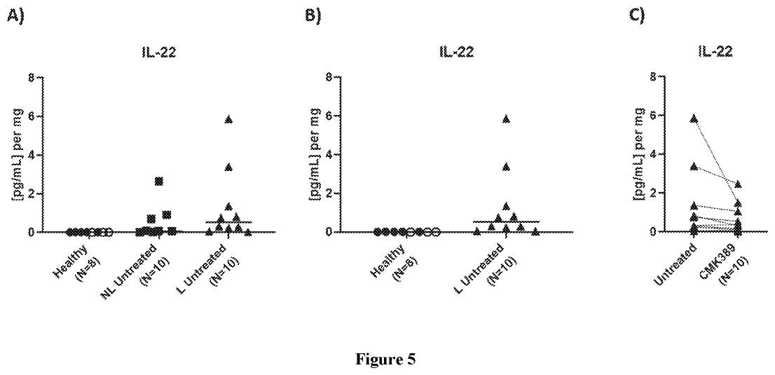

FIG. 5A-B represent relative levels (per mg biopsy) of IL-22 in supernatants of ex vivo untreated cultured skin of healthy people and atopic dermatitis patients (NL=non-lesional, L=lesional). FIG. 5C represents relative levels (per mg biopsy) of IL-22 levels in supernatants of ex vivo cultured skin of atopic dermatitis patients in absence (untreated) or presence of CMK389.

### Conclusion

The reduction of TARC, soluble CD40, IL-22 and IL-24 protein by CMK389 in the culture supernatant of ex vivo cultured skin biopsies of atopic dermatitis patients, all of which have been associated with AD pathophysiology and/or disease activity, indicates that IL-18 antagonists, e.g., CMK389, may provide therapeutic benefit to patients suffering from atopic dermatitis.

### Example 3: A Randomized, Subject and Investigator Blinded, Placebo-Controlled Multicenter Study to Assess the Efficacy and Safety of CMK389 in Patients with Moderate-to-Severe Atopic Dermatitis

To assess the efficacy and safety of CMK389 in patients with moderate to severe atopic dermatitis (AD), a randomized, subject and investigator blinded, placebo-controlled multicenter study is conducted.

### Objectives of the Study:

To assess the efficacy of CMK389 in participants with moderate to severe AD, in particular by looking at IGA (Investigator Global Assessment) response (defined as clear or almost clear and at least 2 point reduction from baseline) at Week 16.
To assess the safety and tolerability of CMK389 in participants with AD, in particular by looking at number, seriousness and frequency of adverse events over time.

The effect of CMK389 and clinical response in participants with moderate to severe AD is to be explored by looking at:
IGA (Investigator Global Assessment) score;
IGA (Investigator Global Assessment) response (defined as clear or almost clear and at least 2 point reduction from baseline) at Week 16;
Change and percent change from baseline of Dermatology Life Quality Index (DLQI) over time;
Patient global impression of change (PGI-c) and severity (PGI-s) over time;
EASI (Eczema Area and Severity Index) score over time assessed as absolute and percent change from baseline;
EASI75 response (defined as ≥75% reduction from baseline in EASI score);
EASI90 response (defined as ≥90% reduction from baseline in EASI score);
Mean NRS (Numerical Rating Scale) pruritus reduction over time;
IGA score over time;
Change and percent change in pruritus from baseline over time using the Pruritus Numerical Rating Scale (NRS).

### Study Design:

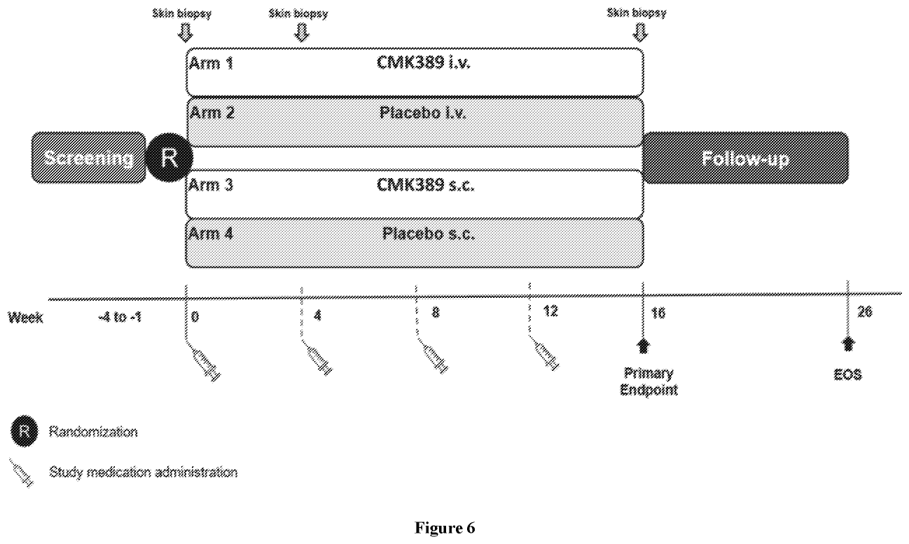

This is a randomized, placebo-controlled, parallel-group, non-confirmatory, investigator and participant blinded study in adult participants with moderate to severe AD. The study consists of up to 4 weeks screening period to assess participants' eligibility, the baseline visit, 4 four weekly administrations of CMK389 within the first 12 weeks of the 16 week treatment period, and an approximately 10-week follow up period which finishes with the end of study visit (EoS). During 16 weeks of treatment period CMK389 or placebo is to be administered intravenously (i.v.) or subcutaneously (s.c.) in monthly intervals (FIG. 6).

Eligible participants with AD are to be randomized into one of four treatment arms (randomization 4:1:2:1):
Arm 1: participants treated i.v. with CMK389;
Arm 2: participants treated i.v. with placebo;
Arm 3: participants treated s.c. with CMK389;
Arm 4: participants treated s.c. with placebo.

Efficacy over time is measured by the clinical scores, such as IGA and additionally EASI and pruritus (as numerical rating scale or NRS). Also, quality of life is measured by Patient reported outomes (PROs); such as Dermatology Life Quality Index (DLQI). Safety is monitored throughout the study by physical exams, vital signs, ECG recordings, adverse events and safety laboratory monitoring.

### Study Population:

The study population consists of adult female and male participants with moderate to severe AD.

### Key Inclusion Criteria:

Adult male or female participants with chronic AD, according to the American Academy of Dermatology Consensus Criteria (Eichenfield et al., J. Am. Acad. Dermatol., 2014, p. 338-51), aged 18 to 65 years, present for at least 1 year before screening.
Moderate to severe AD defined as:
   Investigator Global Assessment (IGA) score of ≥3 (on a scale of 0 to 4, in which 3 is moderate and 4 is severe) at Baseline (or Screening if Baseline is omitted).
   Eczema Area and Severity Index (EASI) score of ≥12 at Baseline (or Screening if Baseline is omitted).
   Pruritus Numerical Rating Scale (NRS) of at least ≥3 at Baseline (or Screening if Baseline is omitted).
Participants who are candidates for a systemic therapy, defined as e.g. inadequate response to treatment with topical medications, or from whom topical treatments are otherwise medically inadvisable (e.g., because of important side effects or safety risks, patients with large affected body surface areas), as assessed by the investigator.
Participants must have a body mass index (BMI) at screening within the range of 18 to ≤35 kg/m². BMI=Body weight (kg)/[Height (m)]²

### Efficacy Assessments

Severity of AD is measured by Clinical Outcomes Assessments (COAs) such as IGA (Investigator Global Assessment); EASI (Eczema area and severity index); Pruritus NRS (Numerical Rating Scale) and in addition Patient Reported Outcomes (PROs) such as DLQI (Dermatology Life Quality Index); PGI-s (Patient's global impression of severity) and PGI-c (Patient's global impression of change). The primary endpoint is IGA response at week 16.

In addition to the clinical parameters, biomarkers will be collected in circulation and in skin.

### IGA

The IGA scale used in the study is vIGA-AD™ (Validated Investigator Global Assessment scale for Atopic Dermatitis). The IGA rating scale is used to determine the severity of AD symptoms and clinical response to treatment. It reflects a participant's overall disease severity for the whole body based on a 5-point scale. The 5-point scale includes: clear, almost clear, mild, moderate, and severe disease (Table 2). IGA response is defined as clear or almost clear after week 16 with at least a 2 point-reduction from baseline.

TABLE 2
Investigator Global Assessment (IGA)

| Score | Morphological Description |
|---|---|
| 0 - Clear | No inflammatory signs of atopic dermatitis (no erythema, no lichenification, no |
| | oozing/crusting). Postinflammatory hyperpigmentation and/or |
| | hypopigmentation may be present. |
| 1 - Almost Clear | Barely perceptible erythema, barely perceptible induration/papulation, and/or |
| | minimal lichenification. No oozing or crusting. |
| 2- Mild | Slight but definite erythema (pink), slight but definite induration/papulation, |
| | and/or slight but definite lichenification. No oozing or crusting. |
| 3 - Moderate | Clearly perceptible erythema (dull red), clearly perceptible |
| | induration/papulation, and/or clearly perceptible lichenification. Oozing and |
| | crusting may be present. |
| 4 - Severe | Marked erythema (deep and bright red), marked induration/papulation, and/or |
| | marked lichenification. Disease is widespread in extent. Oozing or crusting may |
| | be present. |

### EASI

The EASI is used to make an assessment of the extent and severity of AD (Hanifin et al., Exp. Dermatol., 2001, p. 11-8). Each body region (head/neck [H], upper limbs [UL], trunk [T], and lower limbs [LL]) is assessed for:
Severity of AD: the average degree of the following key signs of AD (erythema, induration/papulation, excoriation, and lichenification) will each be assigned a score of 0, 1, 2 or 3 indicating none (0), mild (1), moderate (2), and severe (3) expression of the clinical sign.
Extent of AD: Based on the extent of AD in a particular body region (when each body region is considered as a whole or 100⁰/), an Area score will be assigned to that body region.

### Please Note:

Only inflamed areas should be included in the assessment; dry skin or post inflammatory pigmentation changes should not be included.
The neck is assessed as part of the head region.
The axillae and groin are assessed as part of the trunk.
The buttocks are assessed as part of the lower limbs.

Calculation of BSA (total body surface area affected by AD): Percentage of each body region affected by AD is multiplied by its respective body region corresponding factor (0.1 for head, 0.3 for trunk, 0.2 for upper limbs and 0.4 for lower limbs). Total BSA affected by AD=(0.1×Head area %)+(0.2×Upper limbs area %)+(0.3×Trunk area %)+(0.4×Lower limbs area %).

### Pruritus NRS

Participants will be asked to respond to the question “How would you rate your itch at the worst moment during the previous 24 hours?”. The participant will respond by rating on a 11-point numerical rating scale (NRS) from minimum 0 (no itch) to maximum 10 (worst itch imaginable).

### DLQI

The DLQI or Dermatology Life Quality Index is a 10-item general dermatology disability index designed to assess health-related quality of life (HRQoL) in adult participants with skin diseases such as eczema and is the most frequently used instrument in studies of randomized controlled trials in dermatology (Finlay and Khan, Clin. Exp. Dermatol., 1994, p. 210-6). The measure includes domains of daily activities, leisure, personal relationships, symptoms and feelings, treatment, and work/school. Each item has four response categories ranging from 0 (not at all) to 3 (very much). “Not relevant” is also a valid response and is scored as 0. The DLQI total score is a sum of the 10 questions. Scores range from 0 to 30, with higher scores indicating greater HRQoL impairment.

### PGI-s

PGI-s rates the severity of the current eczema symptoms. “How would you rate your current eczema symptoms?”: 0=none, 1=mild, 2=moderate, 3=severe, 4=very severe.

### PGI-c

PGI-c rates the current eczema symptoms as compared to the start of the study at visits indicated in the assessment schedule. “Compared to the start of this study, how would you rate your current eczema symptoms?”: 1=a great deal worse, 2=moderately worse, 3=a little worse, 4=about the same, 5=a little better, 6=moderately better, 7=a great deal better.

Sequence Correlation Table

| SEQ ID NO: | Identity |
|---|---|
| 1 | Human IL-18 |
| 2 | Cynomolgus IL-18 |
| 3 | H-CDR1 of MOR8775; MOR9464; MOR9441; MOR10222; MOR10579; |
| | MOR9464_N30K; MOR10222_N30S_M54I; MOR9465; MOR9466; |
| 4 | H-CDR2 of MOR8775; |
| 5 | H-CDR3 of MOR8775; MOR9464; MOR9441; MOR10222; MOR10579; |
| | MOR9464_N30K; MOR10222_N30S_M54I; MOR9465; MOR9466; |
| 6 | L-CDR1 of MOR8775; MOR9464; MOR9441; MOR10222; MOR10579; |
| | MOR9464_N30K; MOR10222_N30S_M54I; MOR9465; MOR9466; |
| 7 | L-CDR2 of MOR8775; MOR9464; MOR9441; MOR10222; MOR10579; |
| | MOR9464_N30K; MOR10222_N30S_M54I; MOR9465; MOR9466; |
| 8 | L-CDR3 of MOR8775; MOR9464; MOR9441; MOR10222; MOR10579; |
| | MOR9464_N30K; MOR10222_N30S_M54I; MOR9465; MOR9466; |
| 9 | H-CDR2 of MOR9464; MOR10222; MOR9464_N30K; |
| 10 | H-CDR2 of MOR9441; MOR10579; |
| 11 | H-CDR2 of MOR9465 |
| 12 | H-CDR2 of MOR9466 |
| 13 | H-CDR2 of MOR10222_N30S_M54I; |
| 14 | VH of MOR9464_N30K |
| 15 | Polynucleotide VH of MOR9464_N30K |
| 16 | VL of MOR9464_N30K; MOR9464; MOR8775; MOR9465; MOR9466; |
| | MOR9441 |
| 17 | Polynucleotide VL of MOR9464_N30K |
| 18 | VH of MOR10222_N30S_M54I |
| 19 | Polynucleotide VH of MOR10222_N30S_M54I |
| 20 | VL of MOR10222_N30S_M541; MOR10579; MOR10222 |
| 21 | Polynucleotide VL of MOR10222_N30S_M54I |
| 22 | VH MOR8775; |
| 23 | Polynucleotide VH MOR8775; |
| 24 | Polynucleotide VL MOR8775; |
| 25 | VH MOR9441; |
| 26 | Polynucleotide VH MOR9441; |
| 27 | Polynucleotide VL MOR9441; |
| 28 | VH MOR9464; |
| 29 | Polynucleotide VH MOR9464; |
| 30 | Polynucleotide VL MOR9464; |
| 31 | VH MOR9465; |
| 32 | Polynucleotide VH MOR9465; |
| 33 | Polynucleotide VL MOR9465; |
| 34 | VH MOR9466; |
| 35 | Polynucleotide VH MOR9466; |
| 36 | Polynucleotide VL MOR9466; |
| 37 | VH MOR10579; |
| 38 | Polynucleotide VH MOR10579; |
| 39 | Polynucleotide VL MOR10579; |
| 40 | VH MOR10222; |
| 41 | Polynucleotide VH MOR10222; |
| 42 | Polynucleotide VL MOR10222; |
| 43 | Heavy chain of MOR9464_N30K |
| 44 | Polynucleotide Heavy chain of MOR9464_N30K |
| 45 | Light chain of MOR9464_N30K; MOR9464; MOR8775; MOR9441 |
| 46 | Polynucleotide Light chain of MOR9464_N30K |
| 47 | Heavy Chain MOR9464; |
| 48 | Polynucleotide Heavy Chain MOR9464; |
| 49 | Polynucleotide Light Chain MOR9464; |
| 50 | Heavy Chain MOR9441; |
| 51 | Polynucleotide Heavy Chain MOR9441; |
| 52 | Polynucleotide Light Chain MOR9441; |
| 53 | Heavy Chain MOR10222; |
| 54 | Polynucleotide Heavy Chain MOR10222; |
| 55 | Polynucleotide Light Chain MOR10222; |
| 56 | Heavy Chain MOR8775; |
| 57 | Polynucleotide Heavy Chain MOR8775; |
| 58 | Polynucleotide Light Chain MOR8775; |
| 59 | (Chothia) H-CDR1 MOR9464; |
| 60 | (Chothia) H-CDR2 MOR9464; MOR9464_N30K |
| 61 | (Chothia) H-CDR3 MOR9464; MOR9464_N30K; MOR10222_N30S_M54I |
| 62 | (Chothia) L-CDR1 MOR9464; MOR9464_N30K; MOR10222_N30S_M54I |
| 63 | (Chothia) L-CDR2 MOR9464; MOR9464_N30K; MOR10222_N30S_M54I |
| 64 | (Chothia) L-CDR3 MOR9464; MOR9464_N30K; MOR10222_N30S_M54I |
| 65 | (Chothia) H-CDR1 MOR9464_N30K; |
| 66 | (Chothia) H-CDR1 MOR10222_N30S_M54I |
| 67 | (Chothia) H-CDR2 MOR10222_N30K_M54I; |
| 68 | (Chothia) H-CDR1 MOR13363; |
| 69 | (Chothia) H-CDR2 MOR13363; |
| 70 | (Chothia) H-CDR3 MOR13363; |
| 71 | (Chothia) L-CDR1 MOR13363; |
| 72 | (Chothia) L-CDR2 MOR13363; |
| 73 | (Chothia) L-CDR3 MOR13363; |
| 74 | H-CDR1 of MOR8776; MOR10497; MOR10501; MOR10502; |
| 75 | H-CDR2 of MOR8776; |
| 76 | H-CDR2 of MOR10501 |
| 77 | H-CDR2 of MOR1010502; |
| 78 | H-CDR2 of MOR10497; |
| 79 | H-CDR3 of MOR8776; MOR10497; MOR10501; MOR10502; |
| 80 | L-CDR1 of MOR8776; MOR10497; MOR10501; MOR10502; |
| 81 | L-CDR2 of MOR8776; MOR10497; MOR10501; MOR10502; |
| 82 | L-CDR3 of MOR8776; MOR10497; MOR10501; MOR10502; |
| 83 | VH MOR8776; |
| 84 | Polynucleotide VH MOR8776; |
| 85 | VL MOR8776; MOR10497; MOR10501; MOR10502 |
| 86 | Polynucleotide VL MOR8776; |
| 87 | VH MOR10497; |
| 88 | Polynucleotide VH MOR10497; |
| 89 | Polynucleotide VL MOR10497; |
| 90 | VH MOR10501; |
| 91 | Polynucleotide VH MOR10501; |
| 92 | Polynucleotide VL MOR10501; |
| 93 | VH MOR10502; |
| 94 | Polynucleotide VH MOR10502; |
| 95 | Polynucleotide VL MOR10502; |
| 96 | Heavy Chain of MOR8776; |
| 97 | Polynucleotide Heavy Chain of MOR8776; |
| 98 | Light Chain of MOR8776; MOR10497 |
| 99 | Polynucleotide Light Chain of MOR8776; |
| 100 | Heavy Chain MOR10579; |
| 101 | Polynucleotide Heavy Chain MOR10579; |
| 102 | Polynucleotide Light Chain MOR10579; |
| 103 | Heavy Chain MOR10497; |
| 104 | Polynucleotide Heavy Chain MOR10497; |
| 105 | Polynucleotide Light Chain MOR10497; |
| 106 | H-CDR1 of MOR13363; MOR13361 |
| 107 | H-CDR2 of MOR13363 |
| 108 | H-CDR3 of MOR13363; MOR13361 |
| 109 | L-CDR1 of MOR13363; MOR13361 |
| 110 | L-CDR2 of MOR13363; MOR13361 |
| 111 | L-CDR3 of MOR13363 |
| 112 | VH of MOR13363 |
| 113 | Polynucleotide VH of MOR13363 |
| 114 | VL of MOR13363 |
| 115 | Polynucleotide VL of MOR13363 |
| 116 | Heavy Chain of MOR13363 |
| 117 | Polynucleotide Heavy Chain of MOR13363 |
| 118 | Light Chain of MOR13363 |
| 119 | Polynucleotide Light Chain of MOR13363 |
| 120 | H-CDR1 MOR13341; MOR13342; MOR13347 |
| 121 | H-CDR2 MOR13341; MOR13342; MOR13347 |
| 122 | H-CDR2 MOR13361 |
| 123 | H-CDR3 MOR13341; MOR13342; MOR13347 |
| 124 | L-CDR1 MOR13341; MOR13342; MOR13347 |
| 125 | L-CDR2 MOR13341; MOR13342; MOR13347 |
| 126 | L-CDR3 MOR13361 |
| 127 | L-CDR3 MOR13341 |
| 128 | L-CDR3 MOR13342 |
| 129 | L-CDR3 MOR13347 |
| 130 | VH MOR13341; MOR13342; MOR13347 |
| 131 | Polynucleotide VH MOR13341 |
| 132 | VL MOR13341 |
| 133 | Polynucleotide VL MOR13341 |
| 134 | Heavy chain MOR13341; MOR13342; MOR13347 |
| 135 | Polynucleotide Heavy chain MOR13341 |
| 136 | Light chain MOR13341 |
| 137 | Polynucleotide Light chain MOR13341 |
| 138 | VH MOR13361 |
| 139 | Polynucleotide VH MOR13361 |
| 140 | VL MOR13361 |
| 141 | Polynucleotide VL MOR13361 |
| 142 | Heavy chain MOR13361 |
| 143 | Polynucleotide Heavy chain MOR13361 |
| 144 | Light chain MOR13361 |
| 145 | Polynucleotide Light chain MOR13361 |
| 146 | Polynucleotide VH MOR13342 |
| 147 | VL MOR13342 |
| 148 | Polynucleotide VL MOR13342 |
| 149 | Polynucleotide Heavy chain MOR13342 |
| 150 | Light chain MOR13342 |
| 151 | Polynucleotide Light chain MOR13342 |
| 152 | Polynucleotide VH MOR13347 |
| 153 | VL MOR13347 |
| 154 | Polynucleotide VL MOR13347 |
| 155 | Polynucleotide Heavy chain MOR13347 |
| 156 | Light chain MOR13347 |
| 157 | Polynucleotide Light chain MOR13347 |
| 158 | Heavy Chain of MOR10222_N30S_M54I |
| 159 | Polynucleotide Heavy Chain of MOR10222_N30S_M54I |
| 160 | Light Chain of MOR10222_N30S_M54I; MOR10222; MOR10579 |
| 161 | Polynucleotide Light Chain of MOR10222_N30S_M54I |
| 162 | (Chothia) H-CDR1 MOR10497 |
| 163 | (Chothia) H-CDR2 MOR10497 |
| 164 | (Chothia) H-CDR3 MOR10497 |
| 165 | (Chothia) L-CDR1 MOR10497 |
| 166 | (Chothia) L-CDR2 MOR10497 |
| 167 | (Chothia) L-CDR3 MOR10497 |
| 168 | Heavy chain MOR10501 |
| 169 | Polynucleotide Heavy chain MOR10501 |
| 170 | Light chain MOR10501 |
| 171 | Polynucleotide Light chain MOR10501 |
| 172 | Heavy chain MOR10502 |
| 173 | Polynucleotide Heavy chain MOR10502 |
| 174 | Light chain MOR10502 |
| 175 | Polynucleotide Light chain MOR10502 |
| 176 | Heavy chain MOR9465 |
| 177 | Polynucleotide Heavy chain MOR9465 |
| 178 | Light chain MOR9465 |
| 179 | Polynucleotide Light chain MOR9465 |
| 180 | Heavy chain MOR9466 |
| 181 | Polynucleotide Heavy chain MOR9466 |
| 182 | Light chain MOR9466 |
| 183 | Polynucleotide Light chain MOR9466 |
| 184 | (Chothia) H-CDR1 MOR8776 |
| 185 | (Chothia) H-CDR2 MOR8776 |
| 186 | (Chothia) H-CDR3 MOR8776 |
| 187 | (Chothia) L-CDR1 MOR8776 |
| 188 | (Chothia) L-CDR2 MOR8776 |
| 189 | (Chothia) L-CDR3 MOR8776 |
| 190 | (Chothia) H-CDR1 MOR10501 |
| 191 | (Chothia) H-CDR2 MOR10501 |
| 192 | (Chothia) H-CDR3 MOR10501 |
| 193 | (Chothia) L-CDR1 MOR10501 |
| 194 | (Chothia) L-CDR2 MOR10501 |
| 195 | (Chothia) L-CDR3 MOR10501 |
| 196 | H-CDR2 MOR10502 |
| 197 | (Chothia) H-CDR1 MOR10502 |
| 198 | (Chothia) H-CDR2 MOR10502 |
| 199 | (Chothia) H-CDR3 MOR10502 |
| 200 | (Chothia) L-CDR1 MOR10502 |
| 201 | (Chothia) L-CDR2 MOR10502 |
| 202 | (Chothia) L-CDR3 MOR10502 |
| 203 | (Chothia) H-CDR1 MOR8775 |
| 204 | (Chothia) H-CDR2 MOR8775 |
| 205 | (Chothia) H-CDR3 MOR8775 |
| 206 | (Chothia) L-CDR1 MOR8775 |
| 207 | (Chothia) L-CDR2 MOR8775 |
| 208 | (Chothia) L-CDR3 MOR8775 |
| 209 | (Chothia) H-CDR1 MOR9441 |
| 210 | (Chothia) H-CDR2 MOR9441 |
| 211 | (Chothia) H-CDR3 MOR9441 |
| 212 | (Chothia) L-CDR1 MOR9441 |
| 213 | (Chothia) L-CDR2 MOR9441 |
| 214 | (Chothia) L-CDR3 MOR9441 |
| 215 | (Chothia) H-CDR1 MOR9465 |
| 216 | (Chothia) H-CDR2 MOR9465 |
| 217 | (Chothia) H-CDR3 MOR9465 |
| 218 | (Chothia) L-CDR1 MOR9465 |
| 219 | (Chothia) L-CDR2 MOR9465 |
| 220 | (Chothia) L-CDR3 MOR9465 |
| 221 | (Chothia) H-CDR1 MOR9466 |
| 222 | (Chothia) H-CDR2 MOR9466 |
| 223 | (Chothia) H-CDR3 MOR9466 |
| 224 | (Chothia) L-CDR1 MOR9466 |
| 225 | (Chothia) L-CDR2 MOR9466 |
| 226 | (Chothia) L-CDR3 MOR9466 |
| 227 | (Chothia) H-CDR1 MOR10579 |
| 228 | (Chothia) H-CDR2 MOR10579 |
| 229 | (Chothia) H-CDR3 MOR10579 |
| 230 | (Chothia) L-CDR1 MOR10579 |
| 231 | (Chothia) L-CDR2 MOR10579 |
| 232 | (Chothia) L-CDR3 MOR10579 |
| 233 | (Chothia) H-CDR1 MOR10222 |
| 234 | (Chothia) H-CDR2 MOR10222 |
| 235 | (Chothia) H-CDR3 MOR10222 |
| 236 | (Chothia) L-CDR1 MOR10222 |
| 237 | (Chothia) L-CDR2 MOR10222 |
| 238 | (Chothia) L-CDR3 MOR10222 |
| 239 | H-CDR2 MOR10222_N30S_M54I |
| 240 | (Chothia) H-CDR1 MOR13341 |
| 241 | (Chothia) H-CDR2 MOR13341 |
| 242 | (Chothia) H-CDR3 MOR13341 |
| 243 | (Chothia) L-CDR1 MOR13341 |
| 244 | (Chothia) L-CDR2 MOR13341 |
| 245 | (Chothia) L-CDR3 MOR13341 |
| 246 | (Chothia) H-CDR1 MOR13342 |
| 247 | (Chothia) H-CDR2 MOR13342 |
| 248 | (Chothia) H-CDR3 MOR13342 |
| 249 | (Chothia) L-CDR1 MOR13342 |
| 250 | (Chothia) L-CDR2 MOR13342 |
| 251 | (Chothia) L-CDR3 MOR13342 |
| 252 | (Chothia) H-CDR1 MOR13347 |
| 253 | (Chothia) H-CDR2 MOR13347 |
| 254 | (Chothia) H-CDR3 MOR13347 |
| 255 | (Chothia) L-CDR1 MOR13347 |
| 256 | (Chothia) L-CDR2 MOR13347 |
| 257 | (Chothia) L-CDR3 MOR13347 |
| 258 | (Chothia) H-CDR1 MOR13361 |
| 259 | (Chothia) H-CDR2 MOR13361 |
| 260 | (Chothia) H-CDR3 MOR13361 |
| 261 | (Chothia) L-CDR1 MOR13361 |
| 262 | (Chothia) L-CDR2 MOR13361 |
| 263 | (Chothia) L-CDR3 MOR13361 |

1. An IL-18 antagonist for use in the treatment and/or prevention of atopic dermatitis or a related condition in a subject in need thereof.

2. The IL-18 antagonist for use according to claim 1, wherein the IL-18 antagonist specifically binds IL-18, and wherein the IL-18 antagonist does not bind the IL-18/IL-18 binding protein (IL-18 BP) complex.

3. The IL-18 antagonist for use according to any one of the preceding claims, wherein the IL-18 antagonist is an isolated antibody or antibody fragment.

4. The IL-18 antagonist for use according to any one of the preceding claims, wherein the IL-18 antagonist is a human, humanized or chimeric antibody or antibody fragment.

5. The IL-18 antagonist use according to any one of the preceding claims, wherein the IL-18 antagonist binds human IL-18 with a dissociation constant (KD) of 100 pM or less, e.g., 50 pM or less, 25 pM or less, 10 pM or less, 5 pM or less, preferably with a KD of 0.5 pM to 20 pM, in particular as measured by SET.

6. The IL-18 antagonist for use according to any one of the preceding claims, wherein the IL-18 antagonist inhibits I1-18-induced interferon gamma (IFN-γ) production from KG-1 cells with IC50 of less than 5 nM, e.g., 0.1 to 1 nM.

7. The IL-18 antagonist for use according to any one of the preceding claims, wherein the antibody or the antibody fragment inhibits I1-18-induced interferon gamma (IFN-7) production in whole blood with IC50 of less than 150 nM, e.g., 5 to 10 nM.

8. The IL-18 antagonist for use according to any one of the preceding claims, wherein the IL-18 antagonist comprises: a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8.

9. The IL-18 antagonist for use according to claim 8, wherein IL-18 antagonist comprises a heavy chain variable domain comprising SEQ ID NO: 14 or a sequence at least 90% identical thereto; and a light chain variable domain comprising SEQ ID NO: 16 or a sequence at least 90% identical thereto.

10. The IL-18 antagonist for use according to claim 9, wherein the IL-18 antagonist comprises a mutation in the heavy chain framework, wherein the amino acid asparagine at position 30 is replaced with lysine (N30K; numbering according to Kabat).

11. The IL-18 antagonist for use according to any one of claims 8 to 10, wherein the IL-18 antagonist comprises a heavy chain comprising SEQ ID NO: 43 or a sequence at least 90% identical thereto; and a light chain comprising SEQ ID NO: 45 or a sequence at least 90% identical thereto.

12. The IL-18 antagonist for use according to any one of the preceding claims, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score;
(b) a decrease from baseline in Dermatology Life Quality Index (DLQI);
(c) a decrease from baseline in a patient global impression of severity (PGIS);
(d) a decrease from baseline in a patient global impression of change (PGIC);
(e) a decrease from baseline in Eczema Area and Severity Index (EASI) score; and
(f) a decrease from baseline in Pruritus Numeric Rating Scale (NRS) score.

13. The IL-18 antagonist for use according to claim 12, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score by at least by 2 points, in particular a decrease from baseline in IGA score by at least by 2 points and clear or almost clear status;
(b) a decrease from baseline in Dermatology Life Quality Index (DLQI) of at least 30%, preferably at least 40%;
(c) an improvement from baseline in a patient global impression of severity (PGIS) by at least 1 point;
(d) an improvement from baseline in a patient global impression of change (PGIC) by at least 1 point;
(e) a decrease from baseline in Eczema Area and Severity Index (EASI) score of at least 50%;
(f) percent responders with ≥50% improvement in EASI (EASI50);
(g) percent responders with ≥75% improvement in EASI (EASI75);
(h) percent responders with ≥90% improvement in EASI (EASI90);
(i) percent responders with ≥100% improvement in EASI (EASI100);
(j) a decrease from baseline in Pruritus Numeric Rating Scale (NRS) score by at least 3 points, preferably by at least 4 points.

14. The IL-18 antagonist for use according to any one of the preceding claims, wherein the atopic dermatitis is moderate-to-severe atopic dermatitis.

15. The IL-18 antagonist for use according to claim 14, wherein the moderate to severe atopic dermatitis is characterized by (i) an Investigator's Global Assessment (IGA) score of 3 or 4, and/or (ii) an Eczema Area and Severity Index (EASI) score of at least 10, particularly at least 12.

16. The IL-18 antagonist for use according to any one of the preceding claims, wherein the atopic dermatitis is accompanied by an infection or wherein the related condition is an infection, in particular a skin infection, more particularly a skin superinfection.

17. The IL-18 antagonist for use according to claim 16, wherein the infection is (i) a bacterial infection, e.g., staphylococcal bacteria, such as Staphylococcus (S.) aureus, or streptococcal bacteria, such as Streptococcus (S.) epidermitis, and/or (ii) a viral infection, e.g., herpes viral infection.

18. The IL-18 antagonist for use according to any one of the preceding claims, wherein the subject did not adequately respond to a treatment with a topical atopic dermatitis therapy.

19. The IL-18 antagonist for use according to any one of the preceding claims, wherein the subject was refractory to topical atopic dermatitis therapy or the subject did not adequately respond to a treatment with a topical atopic dermatitis therapy.

20. The IL-18 antagonist for use according to claim 18 or 19, wherein the topical atopic dermatitis therapy is selected from the group consisting of topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs.

21. The IL-18 antagonist for use according to claim 20, wherein the topical steroid is selected from the group consisting of a group I topical corticosteroid (TCS), group II topical corticosteroid (TCS) and group III topical corticosteroid (TCS).

22. The IL-18 antagonist for use according to claim 20, wherein the topical corticosteroid (TCS) is selected from the group consisting of methylprednisolone aceponate, mometasone furoate, fluticasone propionate, betamethasone valerate and hydrocortisone butyrate.

23. The IL-18 antagonist for use according to any one of the preceding claims, wherein the IL-18 antagonist is administered subcutaneously or intravenously to a subject in need thereof.

24. The IL-18 antagonist for use according to any one of the preceding claims, wherein the IL-18 antagonist is administered at a dose sufficient to achieve a therapeutically effective serum level.

25. The IL-18 antagonist for use according to claim 24, wherein the serum level is maintained during the treatment course.

26. The IL-18 antagonist for use according to any one of the preceding claims, wherein the IL-18 antagonist is administered once a week, once every two weeks, once every three weeks, once every four weeks, once every eight weeks, once every 12 weeks, in particular once every 4 weeks.

27. The IL-18 antagonist for use according to any one of the preceding claims, wherein a second therapeutic agent is administered to the subject before, after, or concurrent with the IL-18 antagonist of any one of claims 1 to 11.

28. The IL-18 antagonist for use according to claim 27, wherein the second therapeutic agent is selected from the group consisting of steroid, cyclosporine, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab.

29. The IL-18 antagonist for use according to claim 27, wherein the second therapeutic agent is a low to medium potency steroid, e.g., topical or oral steroid, e.g., corticosteroid.

30. The IL-18 antagonist for use according to claim 29, wherein the second therapeutic agent is selected from the group consisting of a group I topical corticosteroid (TCS), group II topical corticosteroid (TCS) and group III topical corticosteroid (TCS).

31. The IL-18 antagonist for use according to claim 29, wherein the topical corticosteroid (TCS) is selected from the group consisting of methylprednisolone aceponate, mometasone furoate, fluticasone propionate, betamethasone valerate and hydrocortisone butyrate.

32. A method of treatment and/or prevention of atopic dermatitis or a related condition in a subject in need thereof, comprising administering an IL-18 antagonist.

33. The method according to claim 32, wherein the IL-18 antagonist specifically binds IL-18, and wherein the IL-18 antagonist does not bind the IL-18/IL-18 binding protein (IL-18 BP) complex.

34. The method according to claim 32 or 33, wherein the IL-18 antagonist is an isolated antibody or antibody fragment.

35. The method according to any one of claims 32 to 34, wherein the IL-18 antagonist is a human, humanized or chimeric antibody or antibody fragment.

36. The method according to any one of claims 32 to 35, wherein the IL-18 antagonist binds human IL-18 with a dissociation constant (KD) of 100 pM or less, e.g., 50 pM or less, 25 pM or less, 10 pM or less, 5 pM or less, preferably with a KD of 0.5 pM to 20 pM, in particular as measured by SET.

37. The method according to any one of claims 32 to 36, wherein the IL-18 antagonist inhibits I1-18-induced interferon gamma (IFN-γ) production from KG-1 cells with IC50 of less than 5 nM, e.g., 0.1 to 1 nM.

38. The method according to any one of claims 32 to 37, wherein IL-18 antagonist inhibits IL-18-induced interferon gamma (IFN-γ) production in whole blood with IC50 of less than 150 nM, e.g., 5 to 10 nM.

39. The method according to any one of claims 32 to 38, wherein the IL-18 antagonist comprises: a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8.

40. The method according to claim 39, wherein the IL-18 antagonist comprises a heavy chain variable domain comprising SEQ ID NO: 14 or a sequence at least 90% identical thereto; and a light chain variable domain comprising SEQ ID NO: 16 or a sequence at least 90% identical thereto.

41. The method according to claim 40, wherein the IL-18 antagonist comprises a mutation in the heavy chain framework, wherein the amino acid asparagine at position 30 is replaced with lysine (N30K; numbering according to Kabat).

42. The method according to any one of claims 39 to 41, wherein the IL-18 antagonist comprises a heavy chain comprising SEQ ID NO: 43 or a sequence at least 90% identical thereto; and a light chain comprising SEQ ID NO: 45 or a sequence at least 90% identical thereto.

43. The method according to any one of claims 32 to 42, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score;
(b) a decrease from baseline in Dermatology Life Quality Index (DLQI);
(c) a decrease from baseline in a patient global impression of severity (PGIS);
(d) a decrease from baseline in a patient global impression of change (PGIC);
(e) a decrease from baseline in Eczema Area and Severity Index (EASI) score; and
(f) a decrease from baseline in Pruritus Numeric Rating Scale (NRS) score.

44. The method according to any one of claims 32 to 43, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score by at least by 2 points, in particular a decrease from baseline in IGA score by at least by 2 points and clear or almost clear status;
(b) a decrease from baseline in Dermatology Life Quality Index (DLQI) of at least 30%, preferably at least 40%;
(c) an improvement from baseline in a patient global impression of severity (PGIS) by at least 1 point;
(d) an improvement from baseline in a patient global impression of change (PGIC) by at least 1 point;
(e) a decrease from baseline in Eczema Area and Severity Index (EASI) score of at least 50%;
(f) percent responders with ≥50% improvement in EASI (EASI50);
(g) percent responders with ≥75% improvement in EASI (EASI75);
(h) percent responders with ≥90% improvement in EASI (EASI90);
(i) percent responders with ≥100% improvement in EASI (EASI100);
(j) a decrease from baseline in Pruritus Numeric Rating Scale (NRS) score by at least 3 points, preferably by at least 4 points.

45. The method according to any one of claims 32 to 44, wherein the atopic dermatitis is moderate-to-severe atopic dermatitis.

46. The method according to claim 45, wherein the moderate to severe atopic dermatitis is characterized by (i) an Investigator's Global Assessment (IGA) score of 3 or 4, and/or (ii) an Eczema Area and Severity Index (EASI) score of at least 10, in particular at least 12.

47. The method according to any one of claims 32 to 46, wherein the atopic dermatitis is accompanied by an infection or wherein the related condition is an infection, in particular a skin infection, more particularly a skin superinfection.

48. The method according to claim 47, wherein the infection is (i) a bacterial infection, e.g., staphylococcal bacteria, such as Staphylococcus (S.) aureus, or streptococcal bacteria, such as Streptococcus (S.) epidermitis, and/or (ii) a viral infection, e.g., herpes viral infection.

49. The method according to any one of claims 32 to 48, wherein the subject did not adequately respond to a treatment with a topical atopic dermatitis therapy.

50. The method according to any one of claims 32 to 49, wherein the subject was refractory to topical atopic dermatitis therapy or the subject did not adequately respond to a treatment with a topical atopic dermatitis therapy.

51. The method according to claim 49 or 50, wherein the topical atopic dermatitis therapy is selected from the group consisting of topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs.

52. The method according to claim 51, wherein the topical steroid is selected from the group consisting of a group I topical corticosteroid (TCS), group II topical corticosteroid (TCS) and group III topical corticosteroid (TCS).

53. The method according to claim 51, wherein the topical corticosteroid (TCS) is selected from the group consisting of methylprednisolone aceponate, mometasone furoate, fluticasone propionate, betamethasone valerate and hydrocortisone butyrate.

54. The method according to any one of claims 32 to 53, wherein the IL-18 antagonist is administered subcutaneously or intravenously to a subject in need thereof.

55. The method according to any one of claims 32 to 54, wherein the IL-18 antagonist is administered at a dose sufficient to achieve a therapeutically effective serum level.

56. The method according to claim 55, wherein the serum level is maintained during the treatment course.

57. The method according to any one of claims 32 to 56, wherein the IL-18 antagonist is administered once a week, once every two weeks, once every three weeks, once every four weeks, once every eight weeks, once every 12 weeks, in particular once every 4 weeks.

58. The method according to any one of claims 32 to 57, wherein a second therapeutic agent is administered to the subject before, after, or concurrent with the isolated antibody or antibody fragment of claims 1 to 11.

59. The method according to claim 58, wherein the second therapeutic agent is selected from the group consisting of steroid, cyclosporine, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab.

60. The method according to claim 58, wherein the second therapeutic agent is a low to medium potency steroid, e.g., topical or oral steroid, e.g., corticosteroid.

61. The method according to claim 60, wherein the second therapeutic agent is selected from the group consisting of a group I topical corticosteroid (TCS), group II topical corticosteroid (TCS) and group III topical corticosteroid (TCS).

62. The method according to claim 60, wherein the topical corticosteroid (TCS) is selected from the group consisting of methylprednisolone aceponate, mometasone furoate, fluticasone propionate, betamethasone valerate and hydrocortisone butyrate.

63. Use of an IL-18 antagonist for the manufacture of a medicament for treatment and/or prevention of atopic dermatitis or a related condition in a subject in need thereof.

64. Use of an IL-18 antagonist in treatment and/or prevention of atopic dermatitis or a related condition in a subject in need thereof.

65. The use according to claim 63 or 64, wherein the IL-18 antagonist specifically binds IL-18, and wherein the IL-18 antagonist does not bind the IL-18/IL-18 binding protein (IL-18 BP) complex.

66. The use according to any one of claims 63 to 65, wherein the IL-18 antagonist is an isolated antibody or antibody fragment.

67. The use according to any one of claims 63 to 66, wherein the IL-18 antagonist is a human, humanized or chimeric antibody or antibody fragment.

68. The use according to any one of claims 63 to 67, wherein the IL-18 antagonist binds human IL-18 with a dissociation constant (KD) of 100 pM or less, e.g., 50 pM or less, 25 pM or less, 10 pM or less, 5 pM or less, preferably with a KD of 0.5 pM to 20 pM, in particular as measured by SET.

69. The use according to any one of claims 63 to 68, wherein the IL-18 antagonist inhibits I1-18-induced interferon gamma (IFN-γ) production from KG-1 cells with IC50 of less than 5 nM, e.g., 0.1 to 1 nM.

70. The use according to any one of claims 63 to 69, wherein the IL-18 antagonist inhibits I1-18-induced interferon gamma (IFN-γ) production in whole blood with IC50 of less than 150 nM, e.g., 5 to 10 nM.

71. The use according to any one of claims 63 to 70, wherein the IL-18 antagonist comprises: a heavy chain variable region H-CDR1 comprising SEQ ID NO: 3, a heavy chain variable region H-CDR2 comprising SEQ ID NO: 9, a heavy chain variable region H-CDR3 comprising SEQ ID NO: 5, a light chain variable region L-CDR1 comprising SEQ ID NO: 6, a light chain variable region L-CDR2 comprising SEQ ID NO: 7, and a light chain variable region L-CDR3 comprising SEQ ID NO: 8.

72. The use according to claim 71, wherein the IL-18 antagonist comprises a heavy chain variable domain comprising SEQ ID NO: 14 or a sequence at least 90% identical thereto; and a light chain variable domain comprising SEQ ID NO: 16 or a sequence at least 90% identical thereto.

73. The use according to claim 72, wherein the IL-18 antagonist comprises a mutation in the heavy chain framework, wherein the amino acid asparagine at position 30 is replaced with lysine (N30K; numbering according to Kabat).

74. The use according to any one of claims 71 to 73, wherein the IL-18 antagonist comprises a heavy chain comprising SEQ ID NO: 43 or a sequence at least 90% identical thereto; and a light chain comprising SEQ ID NO: 45 or a sequence at least 90% identical thereto.

75. The use according to any one of claims 63 to 74, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score;
(b) a decrease from baseline in Dermatology Life Quality Index (DLQI);
(c) a decrease from baseline in a patient global impression of severity (PGIS);
(d) a decrease from baseline in a patient global impression of change (PGIC);
(e) a decrease from baseline in Eczema Area and Severity Index (EASI) score; and
(f) a decrease from baseline in Pruritus Numeric Rating Scale (NRS) score.

76. The use according to claim 75, wherein the treatment results in an improvement in an atopic dermatitis-associated parameter and wherein the improvement in the atopic dermatitis-associated parameter is selected from the group consisting of:
(a) a decrease from baseline in Investigator's Global Assessment (IGA) score by at least by 2 points, in particular a decrease from baseline in IGA score by at least by 2 points and clear or almost clear status;
(b) a decrease from baseline in Dermatology Life Quality Index (DLQI) of at least 30%, preferably at least 40%;
(c) an improvement from baseline in a patient global impression of severity (PGIS) by at least 1 point;
(d) an improvement from baseline in a patient global impression of change (PGIC) by at least 1 point;
(e) a decrease from baseline in Eczema Area and Severity Index (EASI) score of at least 50%;
(f) percent responders with ≥50% improvement in EASI (EASI50);
(g) percent responders with ≥75% improvement in EASI (EASI75);
(h) percent responders with ≥90% improvement in EASI (EASI90);
(i) percent responders with ≥100% improvement in EASI (EASI100);
(j) a decrease from baseline in Pruritus Numeric Rating Scale (NRS) score by at least 3 points, preferably by at least 4 points.

77. The use according to any one of claims 63 to 76, wherein the atopic dermatitis is moderate-to-severe atopic dermatitis.

78. The use according to claim 77, wherein the moderate to severe atopic dermatitis is characterized by (i) an Investigator's Global Assessment (IGA) score of 3 or 4, and/or
(ii) an Eczema Area and Severity Index (EASI) score of at least 10, in particular at least 10.

79. The use according to any one of claims 63 to 78, wherein the atopic dermatitis is accompanied by an infection or wherein the related condition is an infection, in particular a skin infection, more particularly a skin superinfection.

80. The use according to claim 79, wherein the infection is (i) a bacterial infection, e.g., staphylococcal bacteria, such as Staphylococcus (S.) aureus, or streptococcal bacteria, such as Streptococcus (S.) epidermitis, and/or (ii) a viral infection, e.g., herpes viral infection.

81. The use according to any one of claims 63 to 80, wherein the subject did not adequately respond to a treatment with a topical atopic dermatitis therapy.

82. The use according to any one of claims 63 to 81, wherein the subject was refractory to a topical atopic dermatitis therapy or the subject did not adequately respond to a treatment with a topical atopic dermatitis therapy.

83. The use according to claim 81 or 82, wherein the topical atopic dermatitis therapy is selected from the group consisting of topical steroid, e.g., corticosteroid, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs.

84. The use according to claim 83, wherein the topical steroid is selected from the group consisting of a group I topical corticosteroid (TCS), group II topical corticosteroid (TCS) and group III topical corticosteroid (TCS).

85. The use according to claim 83, wherein the topical corticosteroid (TCS) is selected from the group consisting of methylprednisolone aceponate, mometasone furoate, fluticasone propionate, betamethasone valerate and hydrocortisone butyrate.

86. The use according to any one of claims 63 to 85, wherein the IL-18 antagonist is administered subcutaneously or intravenously to a subject in need thereof.

87. The use according to any one of claims 63 to 86, wherein the IL-18 antagonist is administered at a dose sufficient to achieve a therapeutically effective serum level.

88. The use according to claim 87, wherein the serum level is maintained during the treatment course.

89. The use according to any one of claims 63 to 88, wherein the IL-18 antagonist is administered once a week, once every two weeks, once every three weeks, once every four weeks, once every eight weeks, once every 12 weeks, in particular once every 4 weeks.

90. The use according to any one of claims 63 to 89, wherein a second therapeutic agent is administered to the subject before, after, or concurrent with the isolated antibody or antibody fragment of claims 1 to 11.

91. The use according to claim 90, wherein the second therapeutic agent is selected from the group consisting of steroid, cyclosporine, tacrolimus, cyclophosphamide, azathioprine, methotrexate, mycophenolate mofetil, apremilast, calcineurin inhibitor, e.g., topical calcineurin inhibitor, phosphodiesterase 4 (PDE4) inhibitor, e.g., topical PDE4 inhibitor, e.g. Crisaborole, adrenocorticotropic hormone analogs, dupilumab, etanercept, adalimumab, infliximab, omalizumab, secukinumab.

92. The use according to claim 90, wherein the second therapeutic agent is a low to medium potency steroid, e.g., topical or oral steroid, e.g., corticosteroid.

93. The use according to claim 90, wherein the second therapeutic agent is selected from the group consisting of a group I topical corticosteroid (TCS), group II topical corticosteroid (TCS) and group III topical corticosteroid (TCS).

94. The use according to claim 90, wherein the topical corticosteroid (TCS) is selected from the group consisting of methylprednisolone aceponate, mometasone furoate, fluticasone propionate, betamethasone valerate and hydrocortisone butyrate.
